(19) United States
(12) Patent Application Publication
(10) Pub. No.: US 2004/0266008 A1
(43) Pub. Date: Dec. 30, 2004

(54) Polyene polyketides, processes for their production and their use as pharmaceuticals

(75) Inventors: Brian O. Bachmann, Nashville, TN (US); James B. McAlpine, Montreal (CA); Emmanuel Zazopoulos, Montreal (CA); Chris M. Farnet, Outremont (CA)

Correspondence Address: YWE J. LOOPER, ECOPIA BIOSCIENCES INC., 7290 FREDERICK-BANTING, SAINT-LAURENT, QC, H4S 2A1, CA

(73) Assignee: Ecopia BioSciences, Inc.

(21) Appl. No.: 10/844,547

(22) Filed: May 13, 2004

Related U.S. Application Data
(60) Provisional application No. 60/469,810, filed on May 13, 2003.
(60) Provisional application No. 60/491,516, filed on Aug. 1, 2003.

(51) Int. Cl. C12N 15/88; C07D 213/53; C07D 207/04

(52) U.S. Cl. 435/458; 554/37; 548/571; 546/332

(57) ABSTRACT

This invention relates to a novel class of linear polyene polyketides, their pharmaceutically acceptable salts and derivatives, and to methods for their production. The compounds may be obtained by cultivation of Streptomyces melanosporafaciens species and isolation of the polyene polyketide, followed by optional chemical production of the isolated polyene polyketide. The compounds may also be produced by other known bacteria. The invention further includes the use of these compounds as inhibitors of fungal cell growth and inhibitors of cancer cell growth. Finally, the invention encompasses pharmaceutical compositions comprising these novel polyketide compounds, or their pharmaceutically acceptable salts or derivatives thereof.

### RELATED APPLICATIONS

This application claims priority to U.S. Provisional Applications U.S. Ser. No. 60/469,810 filed May 13, 2003 and U.S. Ser. No. 60/491,516 filed Aug. 1, 2003. The entire teachings of the above provisional applications are incorporated herein by reference.

### FIELD OF THE INVENTION

This invention relates to a novel class of linear polyene polyketides, their pharmaceutically acceptable salts and derivatives, and to methods for their production. The compounds may be obtained by cultivation of Streptomyces species and isolation of the polyene polyketide, followed by optional chemical production of the isolated polyene polyketide. The compounds may also be produced by other known bacteria. The invention further includes the use of these compounds as inhibitors of fungal and bacterial cell growth and inhibitors of cancer cell growth. Finally, the invention encompasses pharmaceutical compositions comprising these novel polyketide compounds, or their pharmaceutically acceptable salts or derivatives thereof.

### BACKGROUND

Polyketides are a diverse class of naturally occurring molecules typically produced by a variety of organisms, including fungi and mycelial bacteria, in particular actinomycetes. Although polyketides have widely divergent structures, they are classified together because they all share a common biosynthetic pathway in which the carbon backbone of these molecules are assembled by sequential, step-wise addition of two carbon or substitued two carbon units referred to as ketides. Polyene polyketides comprise a chain of ketide units that have been strung together by a series of enzymatic reactions by multimodular polyketide synthase proteins.

Polyketides are usually found in their natural environment only in trace amounts. Moreover, due to their structural complexity, poyketides are notoriously difficult to synthesize chemically. Nevertheless, many polyketides have been developed into effective drugs for the treatment of conditions such as bacterial and fungal infections, cancer and high cholesterol. Adriamycin, zithromax, zocor and nystatin are but a few examples of polyketide molecules, which have been developed into valuable pharmaceuticals. Linearmycin A, having a 60 carbon chain and a degree of unsaturation of 15, is an example of a linear polyene polyketide reported to possess antifungal and antibacterial activity (Sakuda et al., Tetrahedron Letters. Vol. 36, No. 16, 2777-2870 (1995); Sakuda et al., J. Chem Soc., Perkin Trans. 1,2315-2319 (1996)).

Although large numbers of therapeutically important polyketides have been identified, there remains a need to obtain novel polyketides that have enhanced properties or possess completely novel bioactivities. The complex polyketides produced by modular polyketide synthases are particularly valuable, in that they include compounds with known utility as antihelminthics, insecticides, immunosuppressants, cytotoxic, antifungal or antibacterial agents. Because of their structural complexity, such novel polyketides are not readily obtainable by total chemical synthesis. The present invention addresses this need by providing a new class of polyketide compounds with therapeutic activity.

### SUMMARY OF THE INVENTION

The compounds of the present invention may be represented by Formula I:

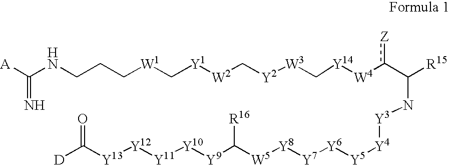

wherein,

A is selected from NHC(O)R¹, N═CR² R³, or NHR³, wherein R¹, R² and R³ are independently selected from the group consisting of C₁₋₆ alkyl, C₂₋₆ alkenyl, C₃₋₆ cycloalkyl, C₃₋₆ heterocycloalkyl, aryl and heteroaryl, wherein said alkyl, alkenyl, cycloalkyl, heterocycloalkyl, aryl and heteroaryl are optionally substituted with a group selected from halogen, oxo, OH, C₂₋₆ alkenyl, NO₂, NH₂, cycloalkyl, heteroaryl or aryl, said C₂₋₆ alkenyl, heteroaryl and aryl being optionally further substituted with one or more groups independently selected from halogen, OH, C₁₋₃ alkyl NO₂ or NH₂;

B is selected from

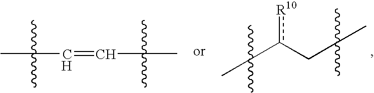

wherein R¹⁰ is OH, —OS(O)₂OH or when the dotted line is a bond then R¹⁰ is oxo;

D is selected from OH or C₁₋₆alkoxy optionally substituted with 1 to 2 phenyl groups, wherein the phenyl group is optionally substituted with C₁₋₆ alkyl or halo;

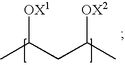

W¹ and W² is each independently selected from

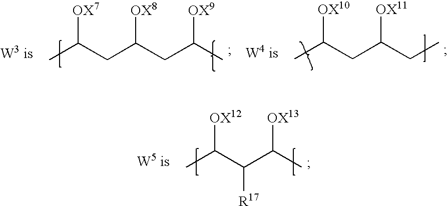

X¹, X², X⁷, X⁸, X⁹, X¹⁰, X¹¹, X¹² and X¹³ is each independently selected from H, —C(O)—R⁷ and a bond such that when any of two neighboring X¹, X², X⁷, X⁸, X⁹, X¹⁰, X¹¹, X¹² and X¹³ is a bond then the two neighboring oxygen atoms and their attached carbon atoms together form a six-membered acetal ring of the formula:

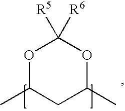

wherein R⁵, R⁶ and R⁷ is each independently selected from H, C₁₋₆ alkyl, C₂₋₇ alkenyl;

Y¹, Y², Y³, Y⁴, Y⁵, Y⁶, Y⁷, Y⁸, Y⁹, Y¹⁰, Y¹¹, Y¹², Y¹³ and Y¹⁴ is each independently selected from —CH₂—CH₂—, —CH═CH—,

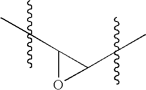

or —CH(OH)—CH(OH)—, wherein any carbon for this selection is optionally substituted with a methyl group;

Z is selected from OH, C₃₋₆ cycloalkyl, C₃₋₆ heterocycloalkyl, NHR⁸,

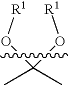

and when the dotted line is a bond then Z is oxo, or NC₁₋₆ alkyl, wherein R⁸ is selected from H, C₁₋₆ alkyl, C₂₋₆alkenyl or C₃₋₆cycloalkyl; and

R¹⁵, R¹⁶ and R¹⁷ is each independently selected from H or CH₃; or a pharmaceutically acceptable salt thereof.

In another aspect, the invention provides compounds of Formula I wherein Z is oxo and all other groups are as previously defined. In one embodiment, the invention provides compounds of Formula I, wherein Z is oxo, A is

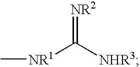

and all other groups are as previously defined. In another embodiment, invention provides compounds of Formula I, wherein Z is oxo, A is

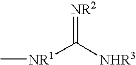

and B is

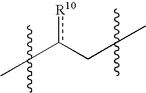

and all other groups are as previously defined; within this aspect R¹⁰ is —OS(O)₂OH. In an additional aspect of this embodiment R¹⁵, R¹⁶ and R¹⁷ are each independently CH₃. In a further aspect of this embodiment the invention provides compounds of Formula I, wherein Z is oxo, A is

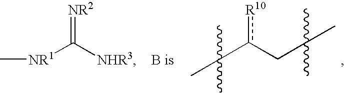

and all other groups are as previously defined; within this aspect R¹⁰ is OH. In an additional aspect of this embodiment R¹⁵, R¹⁶ and R¹⁷ are each independently CH₃. Pharmaceutically acceptable salts of these embodiments are also included within the scope of the invention.

The invention further provides compounds of Formula I, wherein D is OH; and all other groups are as previously defined; or a pharmaceutically acceptable salt thereof. In an aspect of this embodiment, the invention provides compounds of Formula I, wherein D is OH and Z is oxo; and all other groups are as previously defined; or a pharmaceutically acceptable salt thereof. In a further aspect of this embodiment, the invention provides compounds of Formula I wherein D is OH, Z is oxo and A is

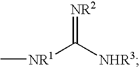

and all other groups are as previously defined; or a pharmaceutically acceptable salt thereof.

The invention further provides polyene polyketides of Formula II below:

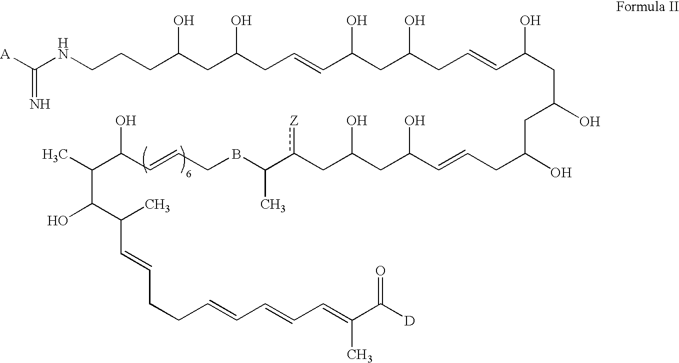

wherein A, B, D and Z are as described in any one of the embodiments above.

The following are exemplary compounds of the invention:

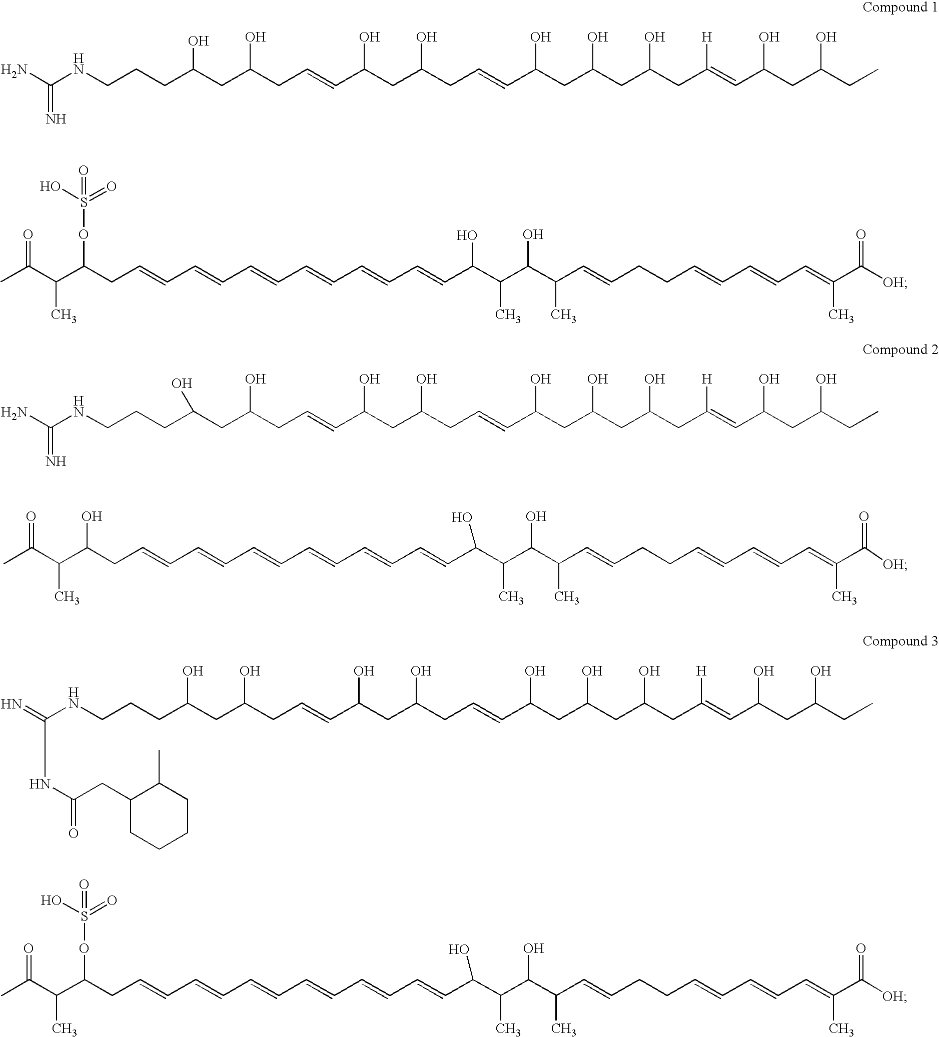

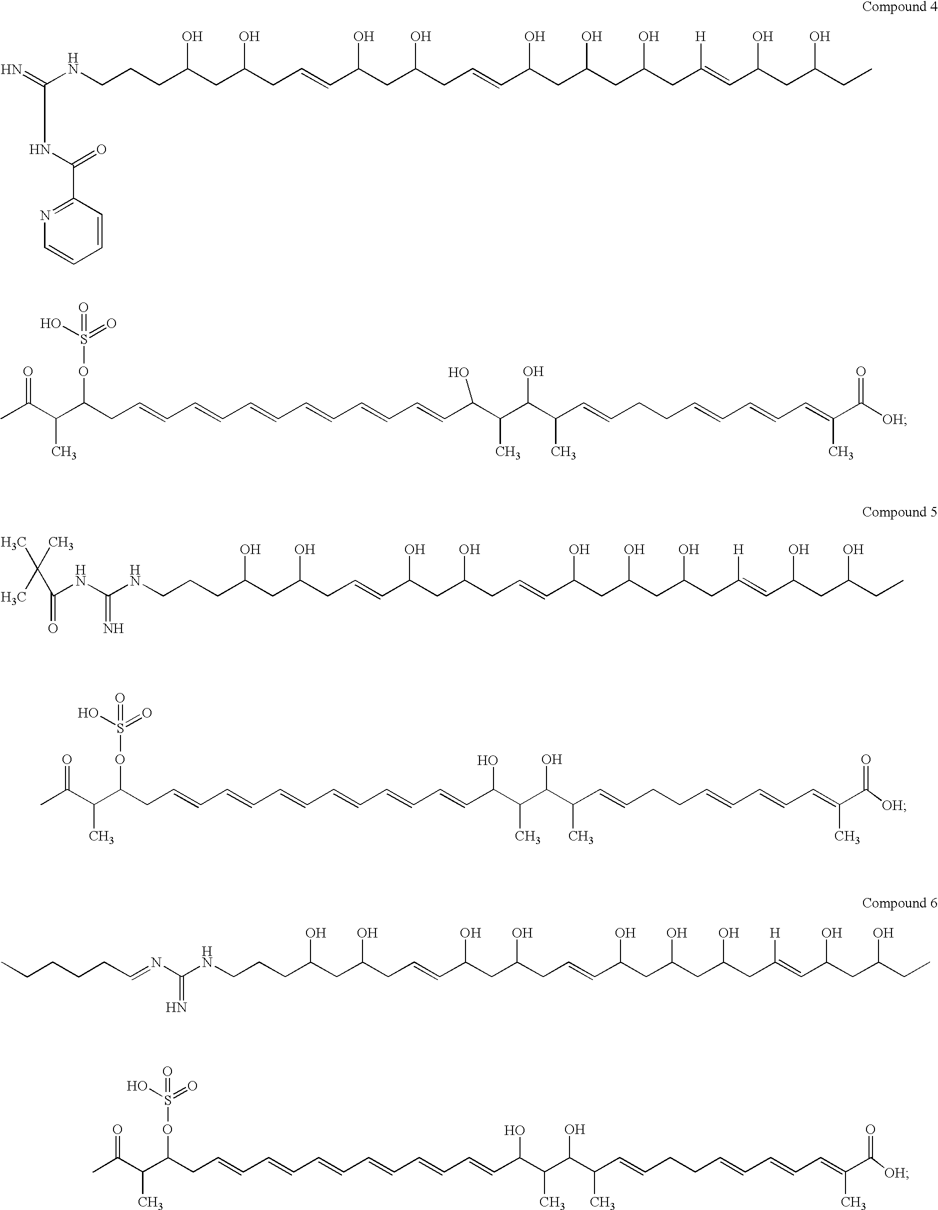

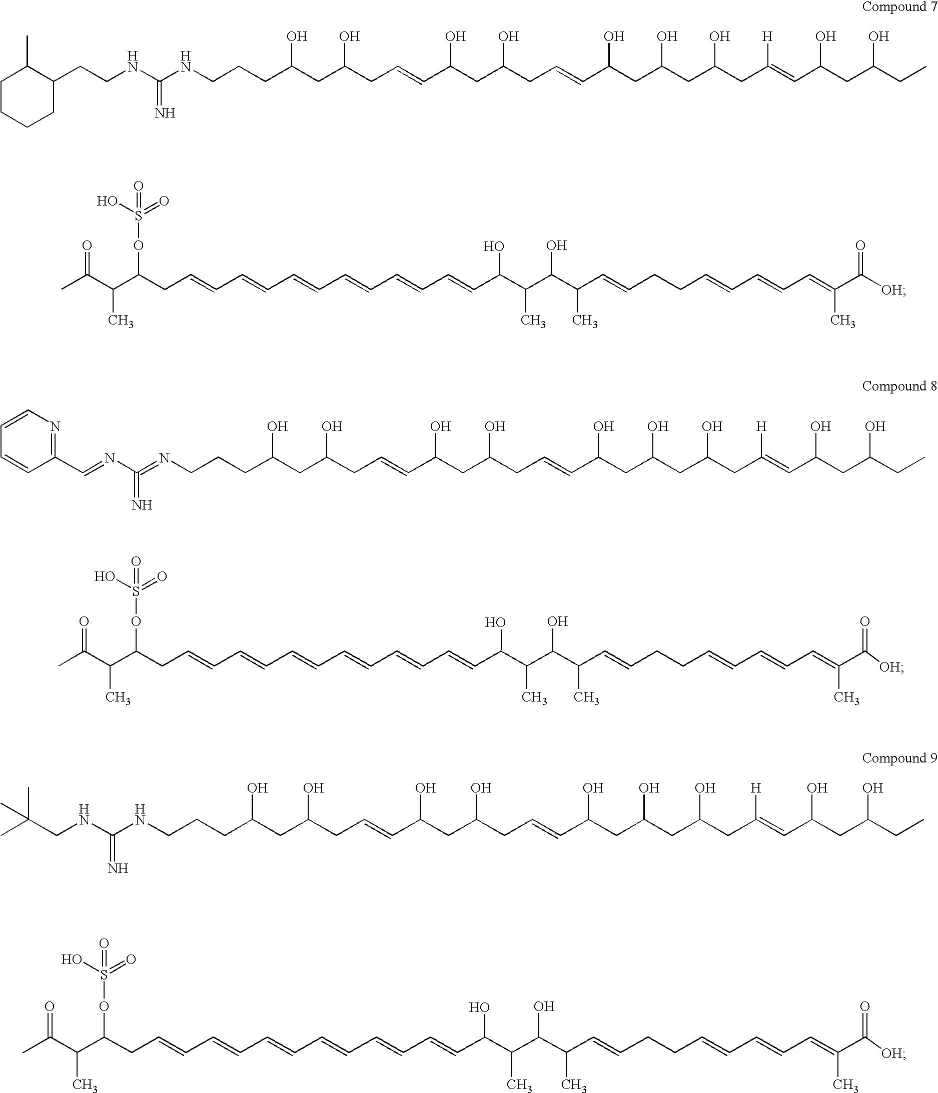

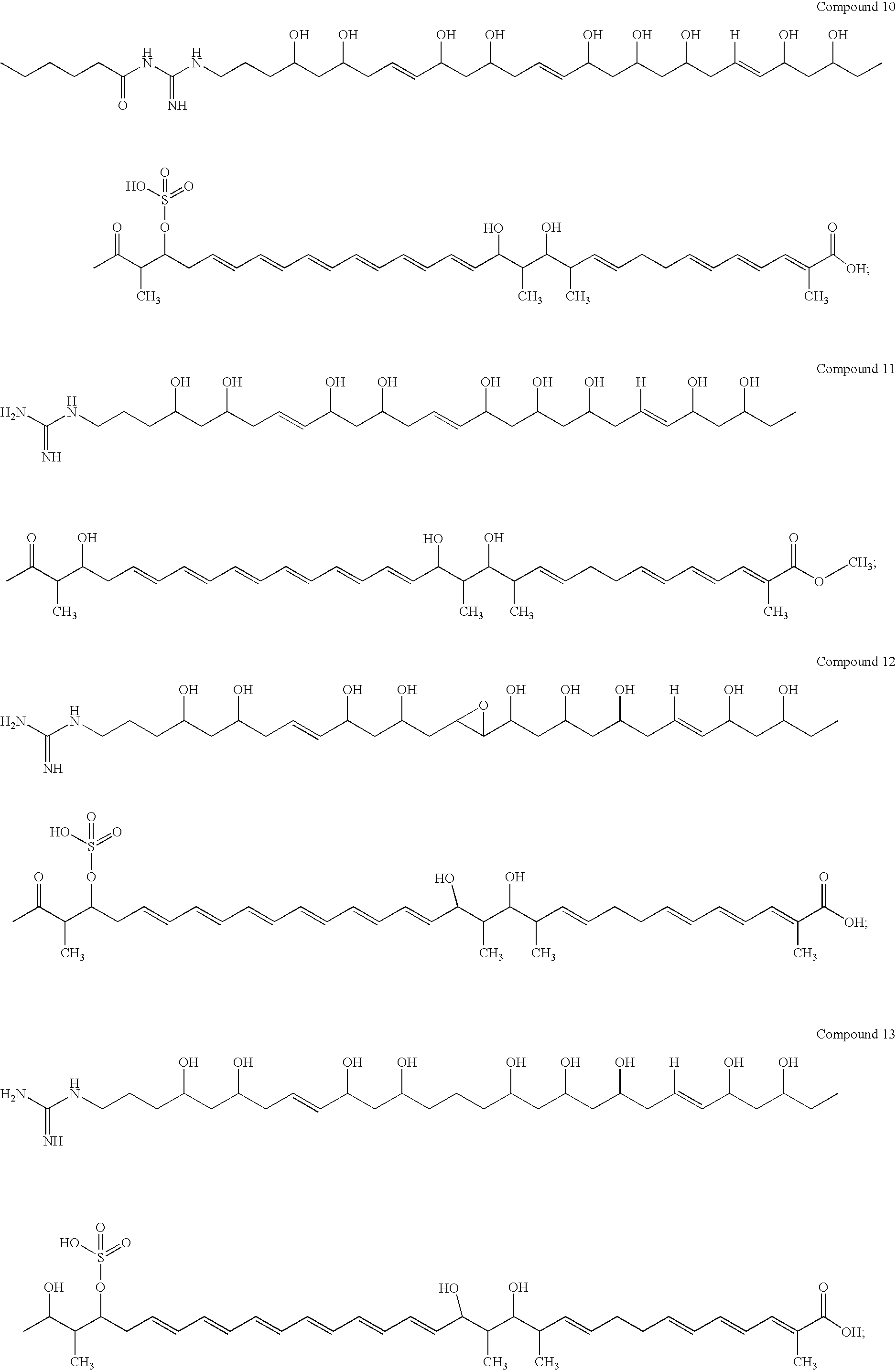

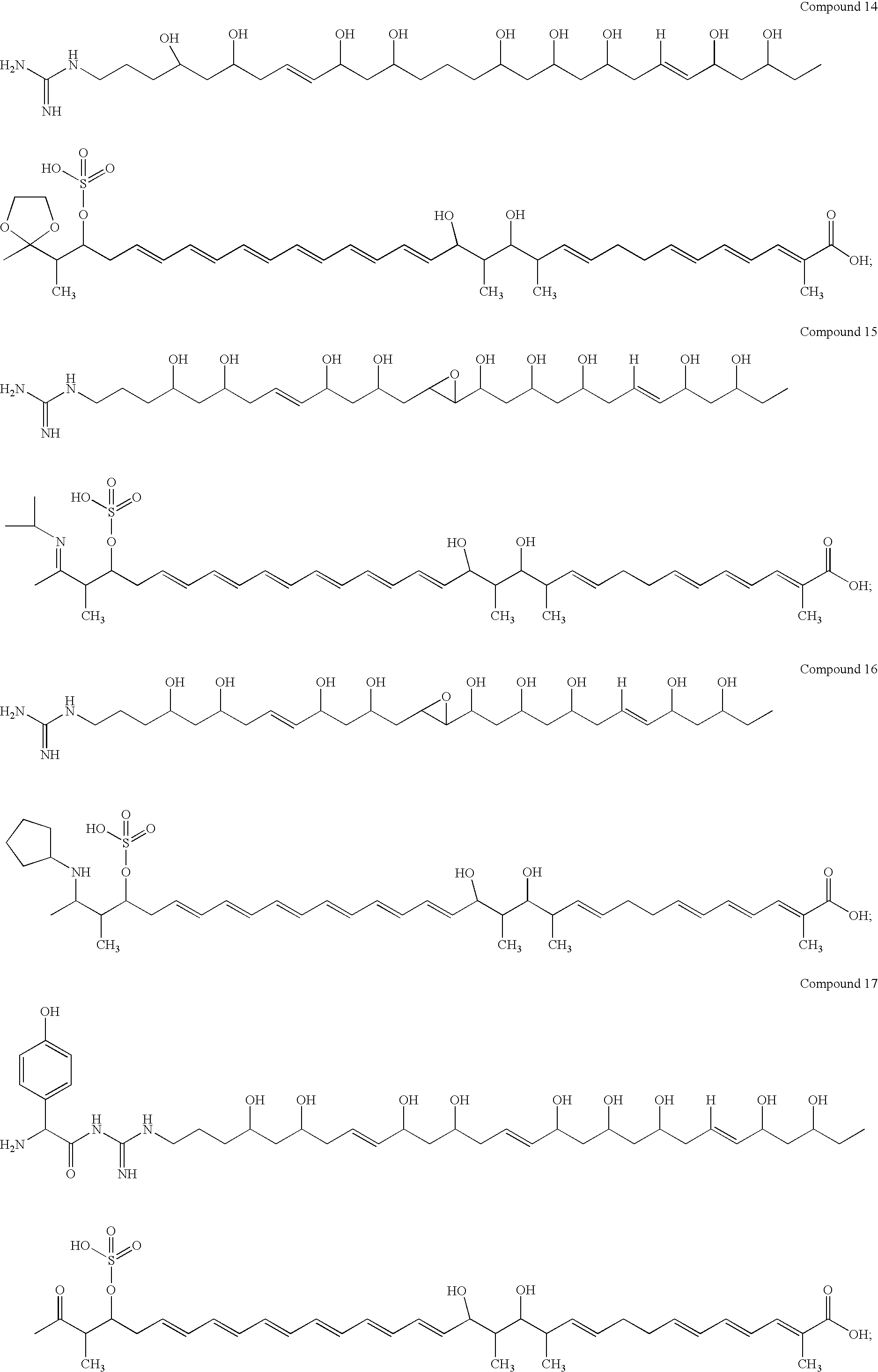

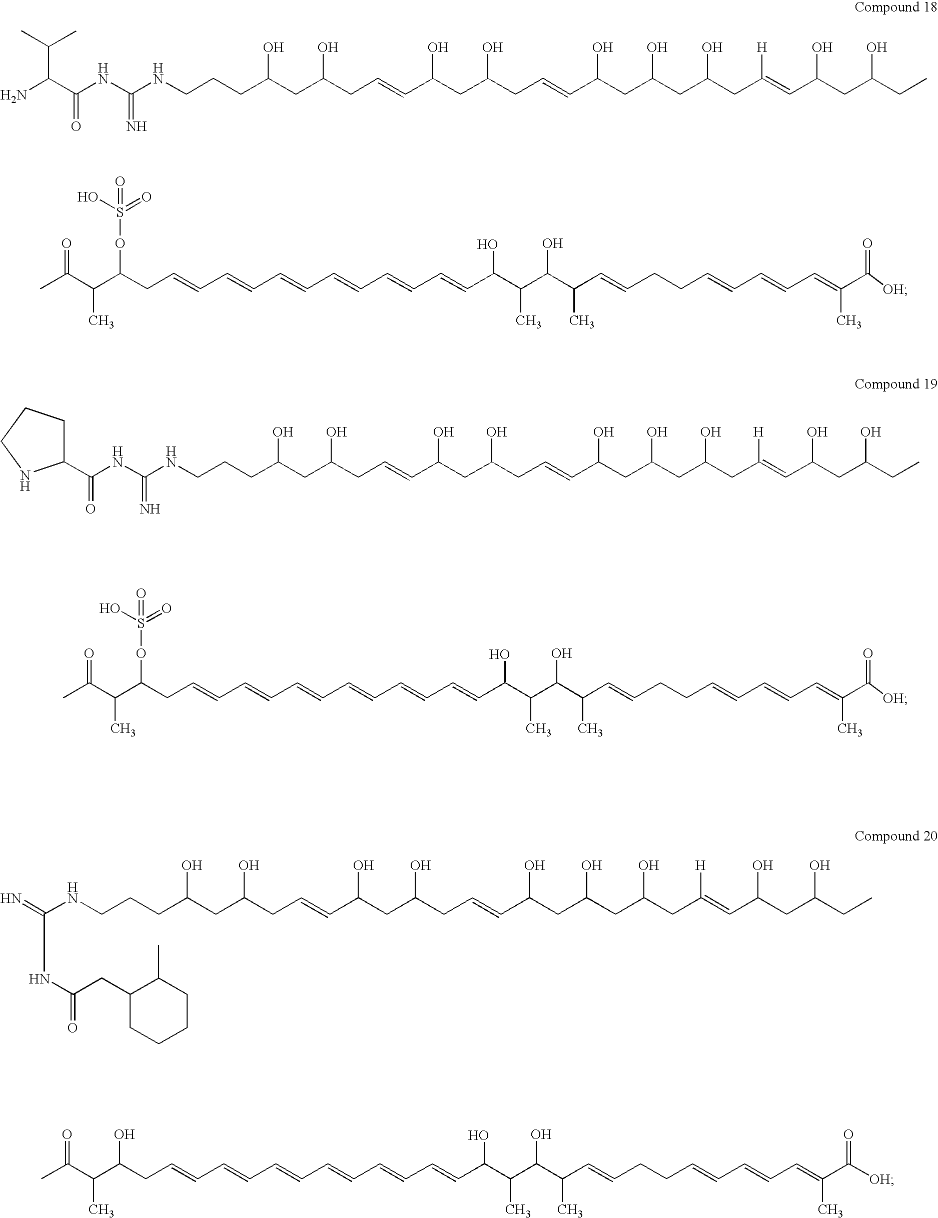

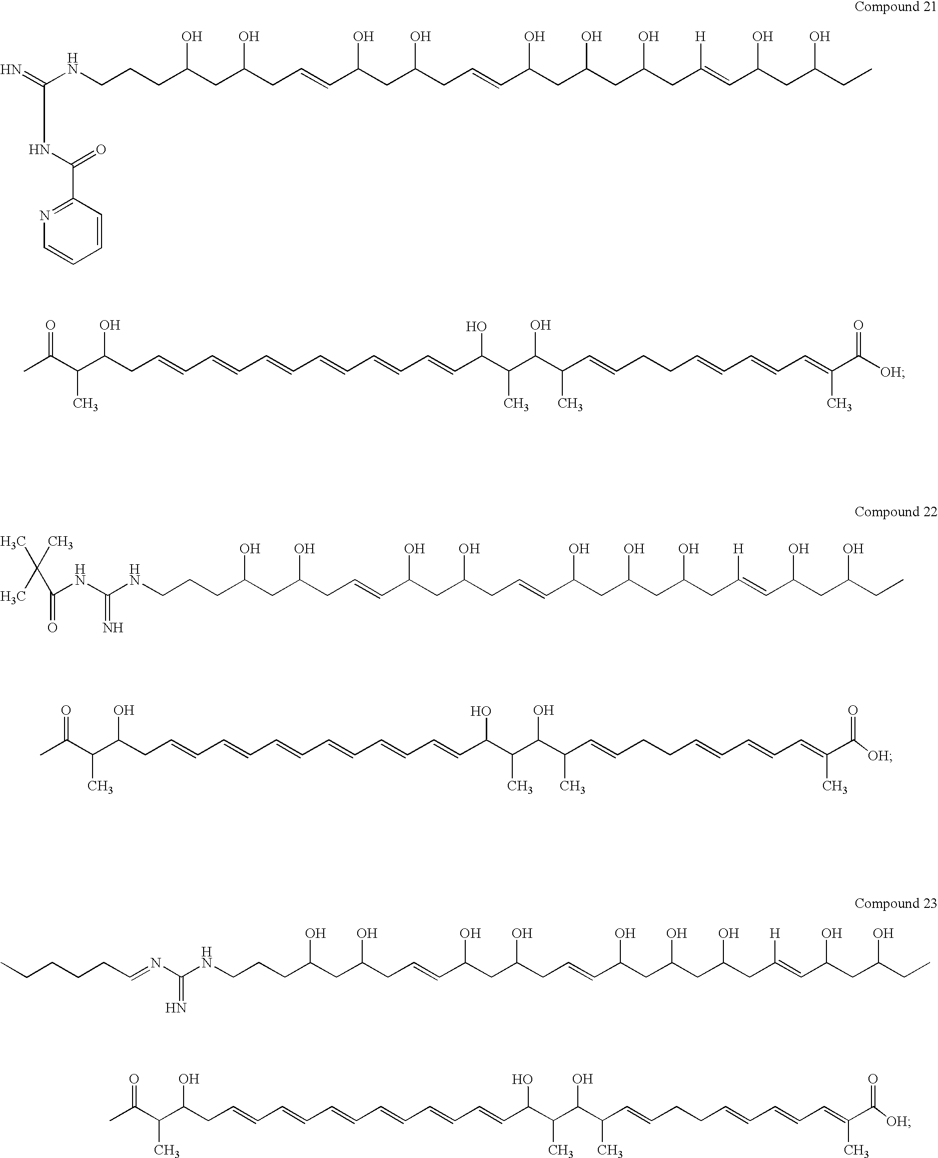

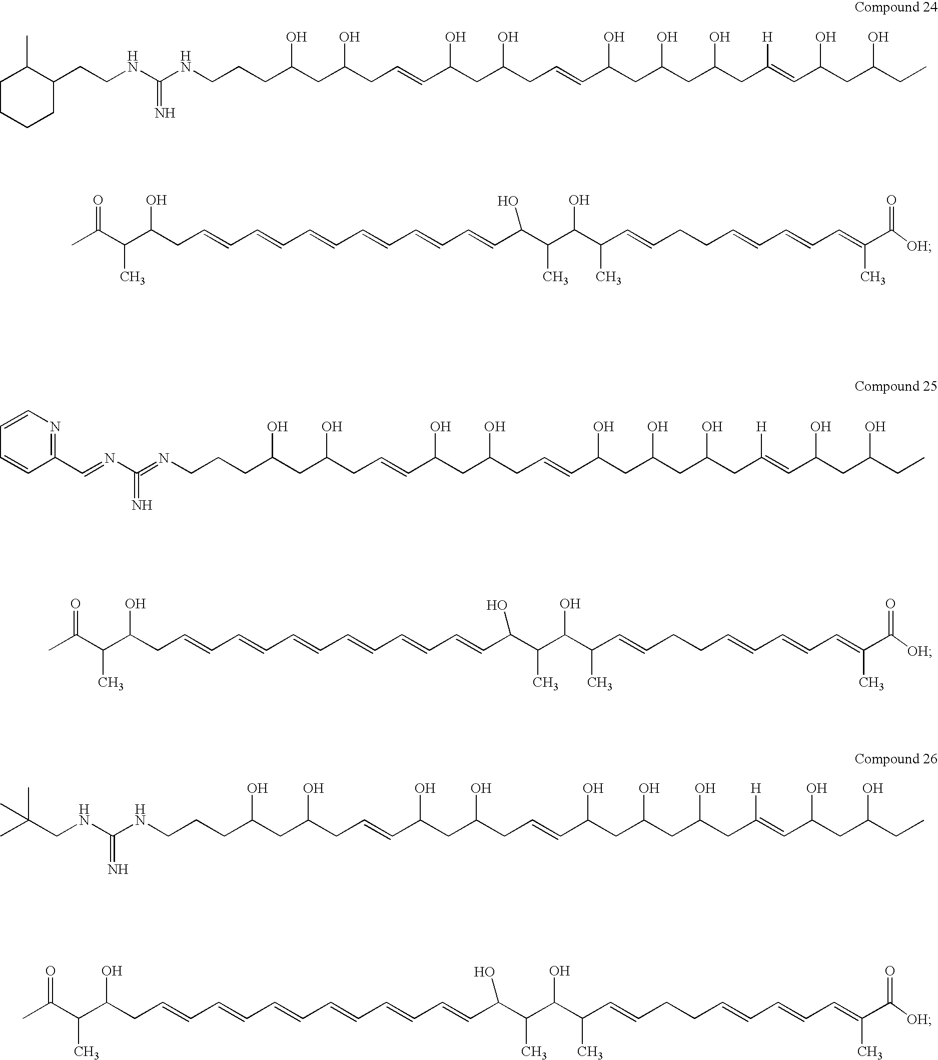

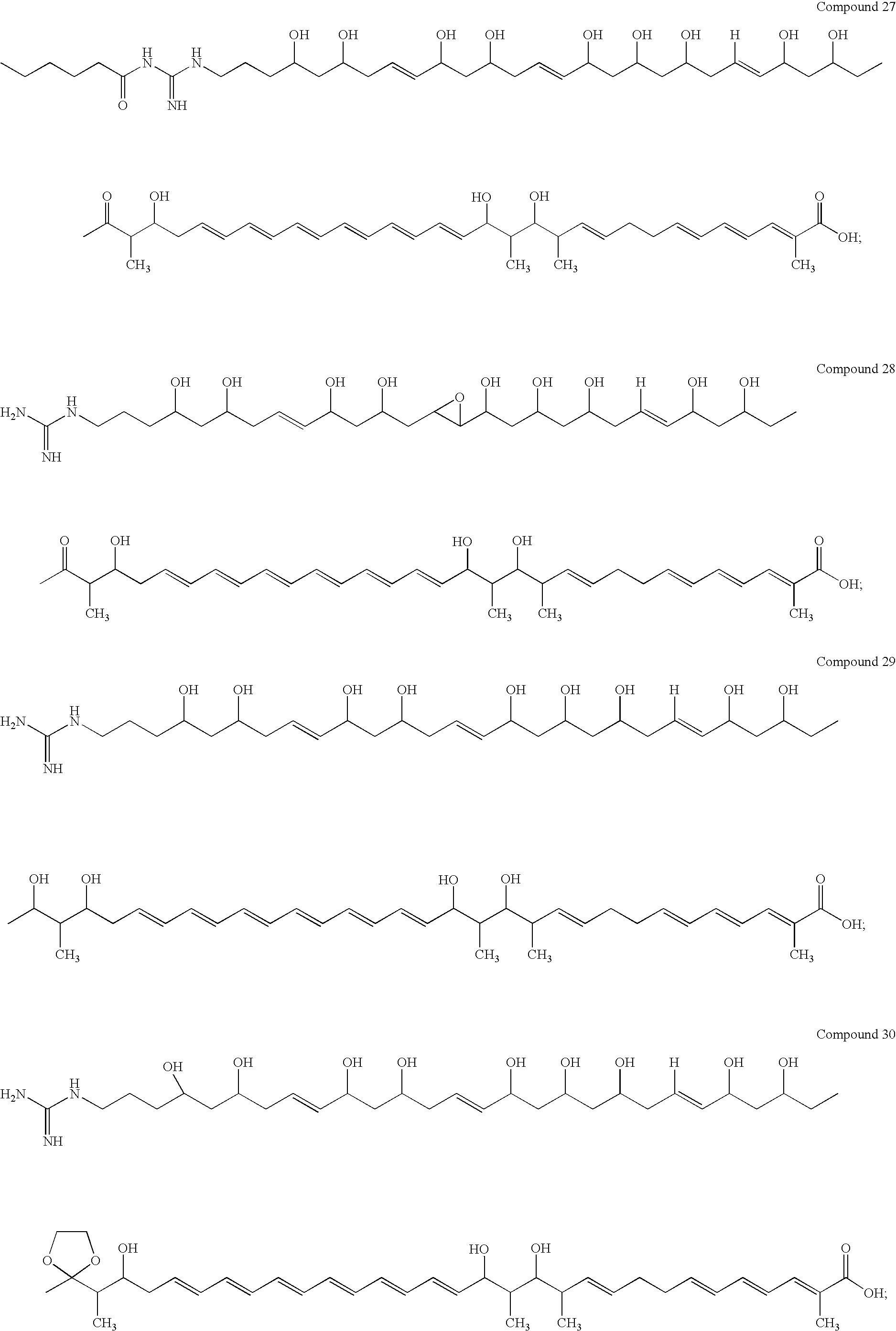

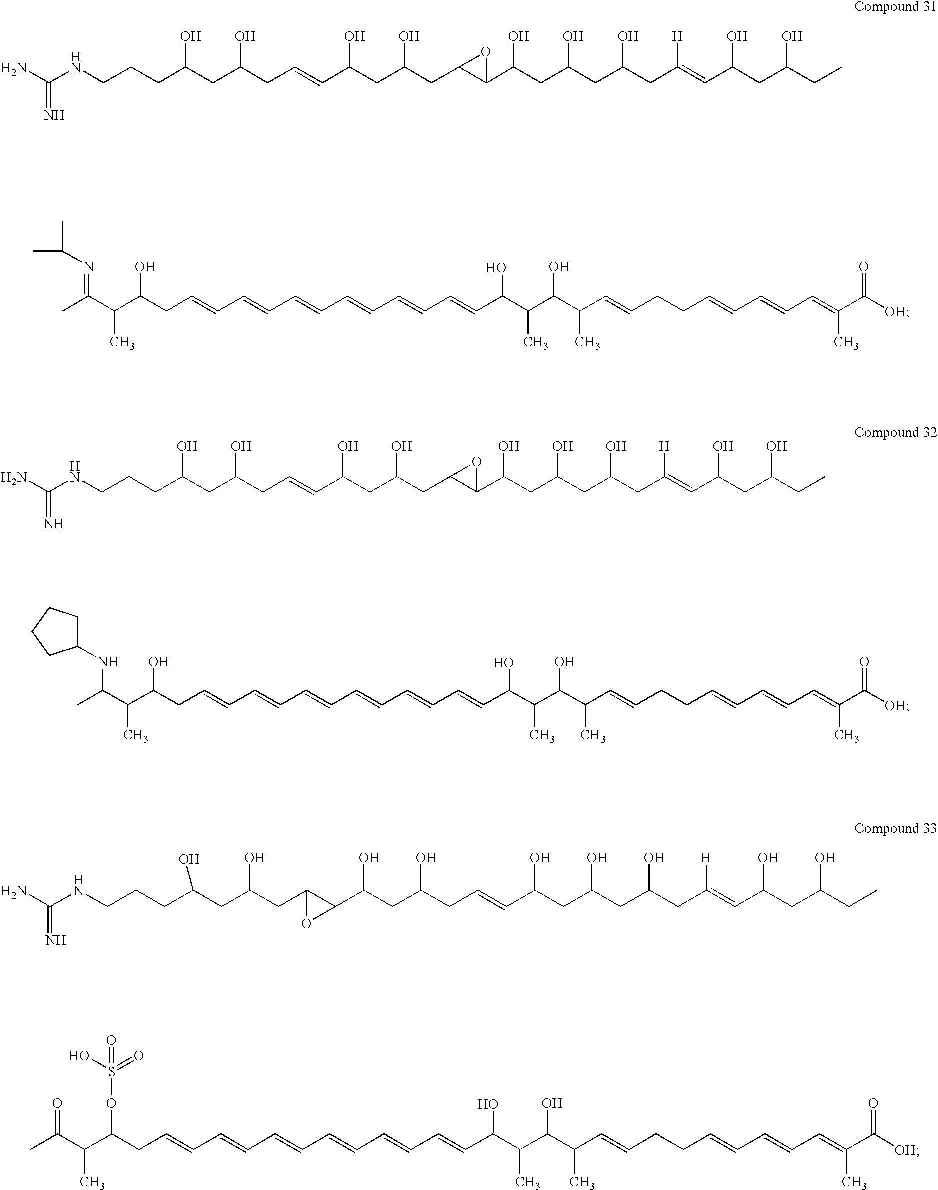

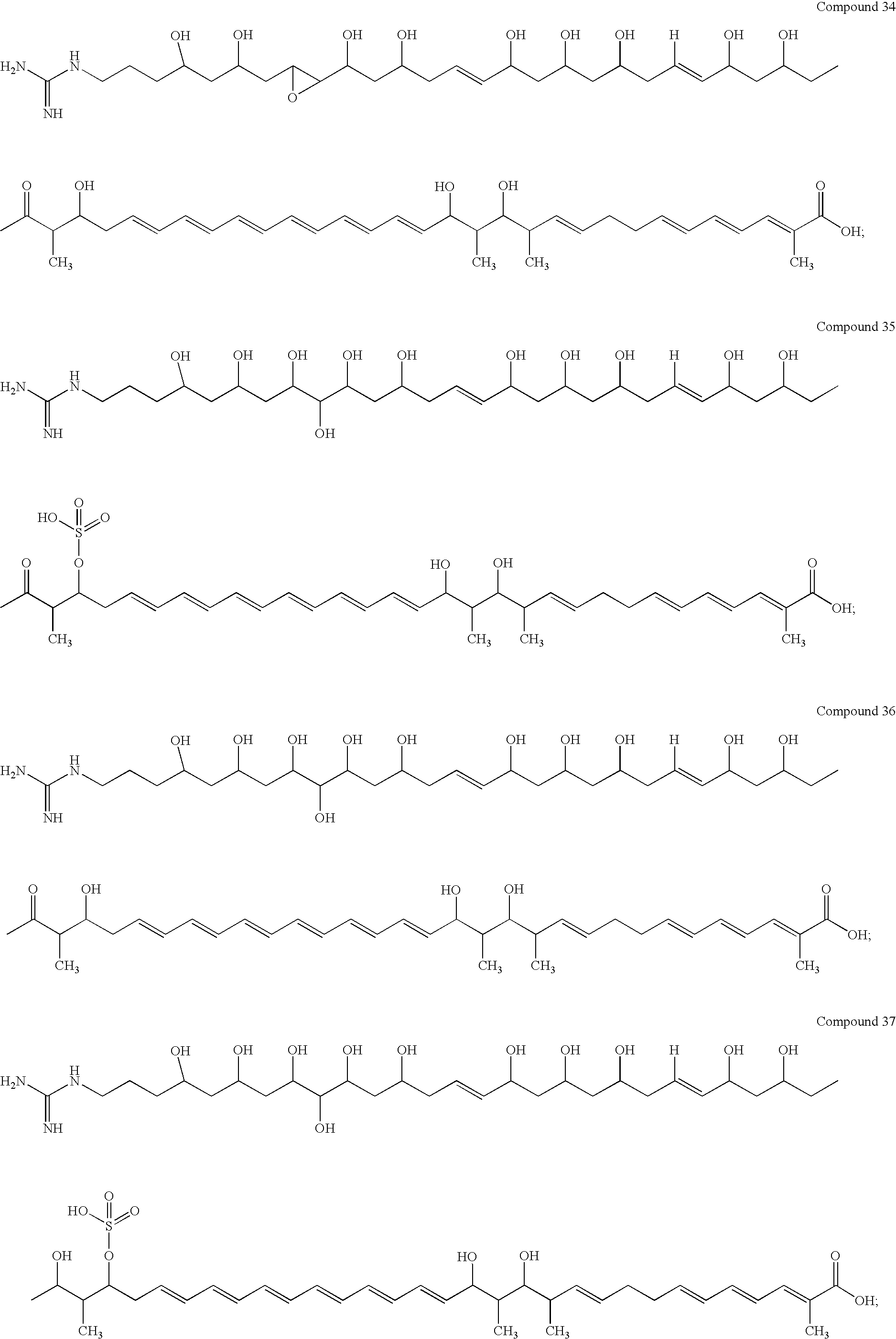

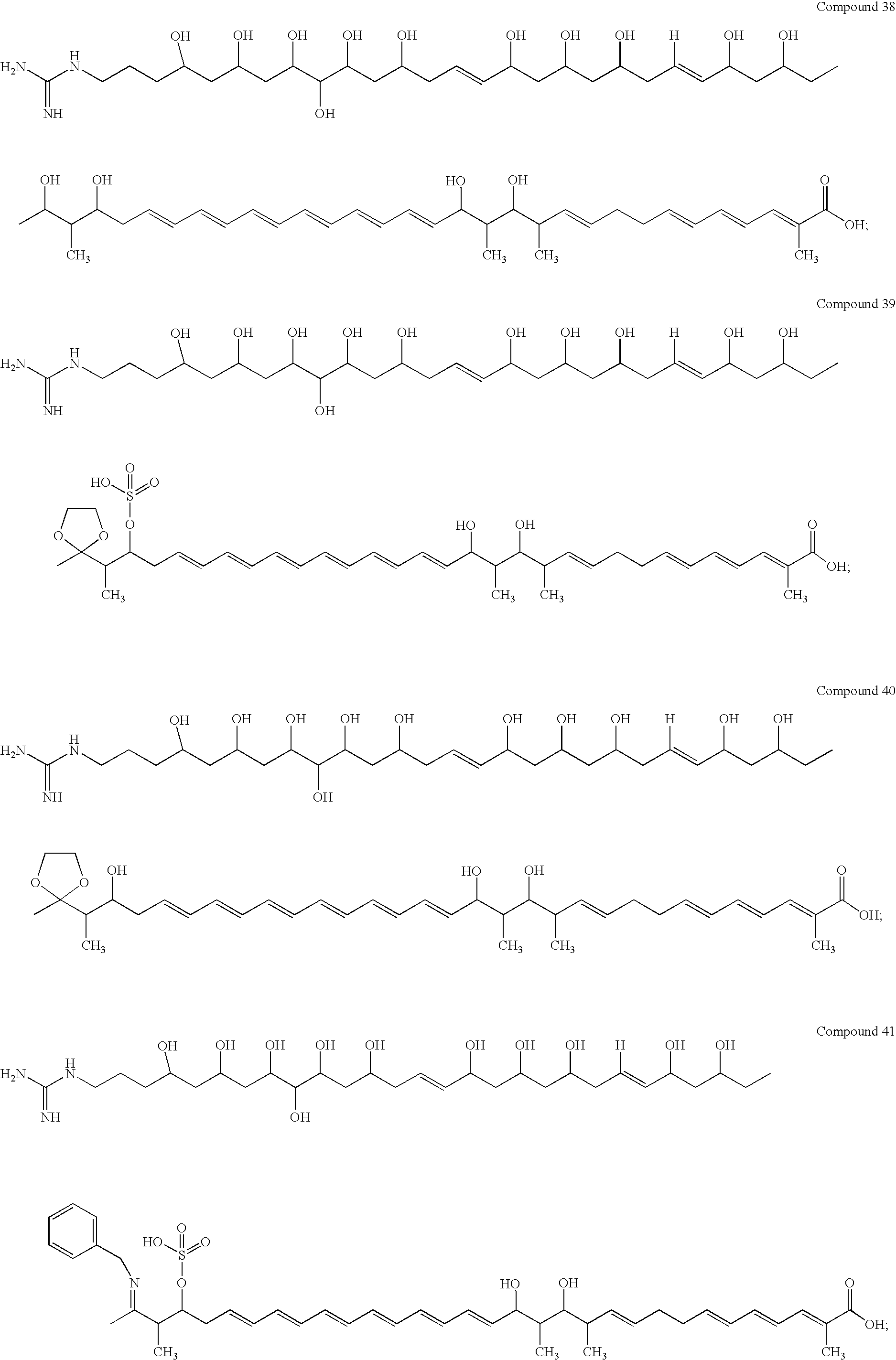

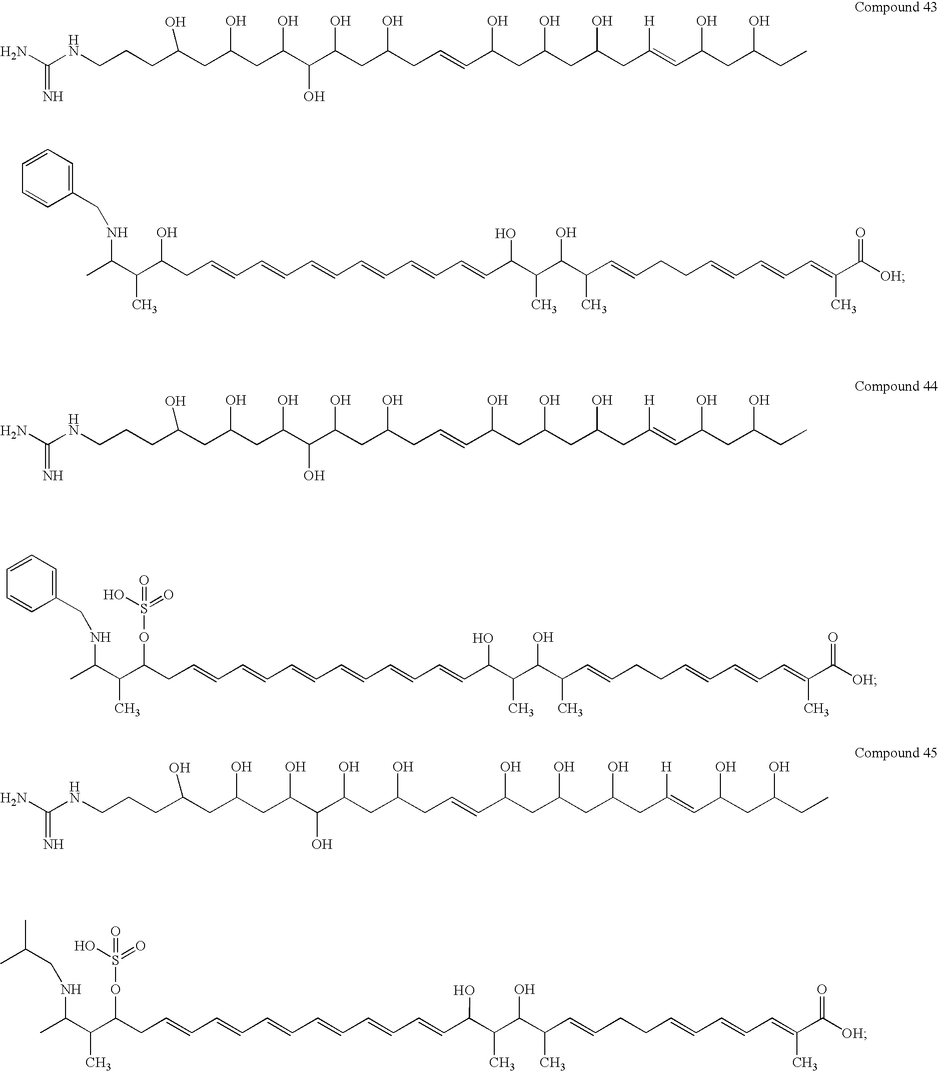

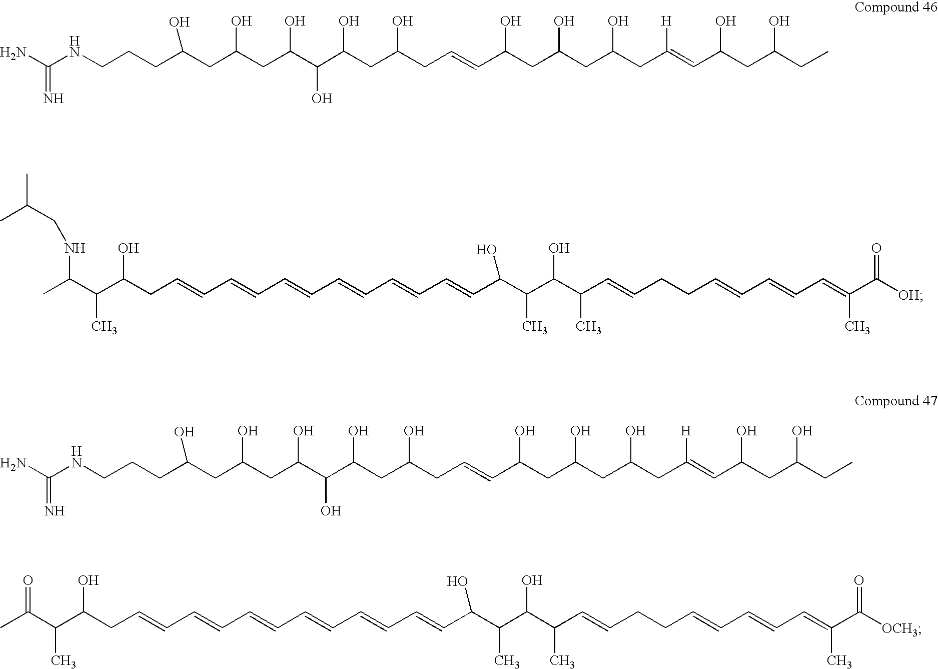

or a pharmaceutically acceptable salt of any one of Compounds 1-47.

In one embodiment, the invention provides Compound 1 of the formula:

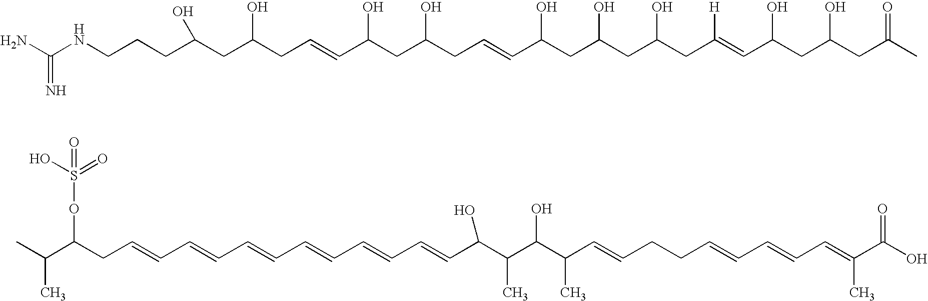

and a pharmaceutically acceptable carrier.

In another embodiment, the invention provides Compound 2 of the formula:

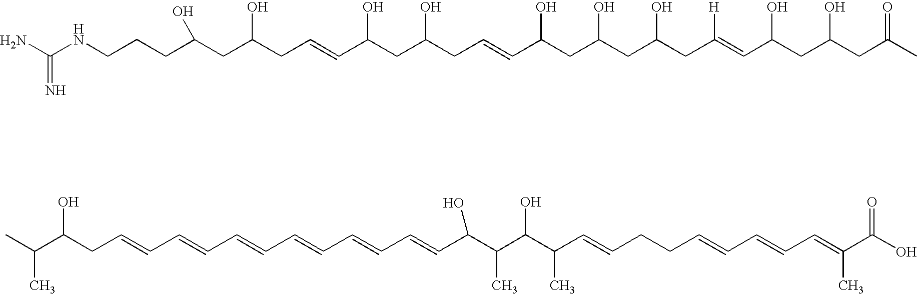

and a pharmaceutically acceptable carrier.

The invention provides pharmaceutical compositions comprising a polyene polyketide compound of Formula I or II, or a pharmaceutically acceptable salt thereof, together with a pharmaceutically acceptable carrier. In one embodiment, the invention relates to a pharmaceutical composition comprising a therapeutically effective amount of Compound 1, Compound 2 or a pharmaceutical acceptable salt of Compound 1 or 2, together with a pharmaceutically acceptable carrier.

The invention further provides a polyene polyketide obtained by a method comprising: (a) cultivating a Streptomyces strain under aerobic conditions in a nutrient medium comprising at least one source of carbon atoms and at least one source of nitrogen atoms, and (b) isolating a compound of Formula I or II from the bacteria cultivated in (a). In one embodiment, the strain is Streptomyces melanosporafaciens NRRL B-12234 or a mutant thereof. In another embodiment, the polyene polyketide is Compound 1 or Compound 2. In a further embodiment, the nutrient medium is selected from the media of Table 1. In a further embodiment, the polyene polyketide generates the ¹H NMR spectra essentially as shown in FIG. 3. In a further embodiment the polyene polyketide generates the ¹H NMR spectra essentially as shown in FIG. 10.

The invention further provides a method for producing a polyene polyketide comprising cultivation of a Streptomyces sp. strain in a nutrient medium comprising at least one source of carbon atoms and at least one source of nitrogen atoms, and isolation and purification of the polyene polyketide. In one embodiment, the polyene polyketide is a compound of Formula I or II. In another embodiment, the polyene polyketide is Compound 1 or Compound 2. In a further embodiment, the polyene polyketide is a derivative or structural analog of Compound 1 or Compound 2, obtained by post synthesis chemical modification of Compound 1 or Compound 2. In a further embodiment, the strain is a Streptomyces melanosporafaciens. In a further embodiment, the strain is Streptomyces melanosporafaciens NRRL B-12234 or a mutant thereof. In a further embodiment, the carbon and nitrogen source is selected from the components of Table 1. In a further embodiment, the nutrient medium selected from the media of Table 1. In a further embodiment, the cultivation is carried out at a temperature ranging from about 18° C. to about 40° C., preferably between 18° C. and 30° C. In a further embodiment, the cultivation is carried out at a pH ranging from about 6 to about 9. In a further embodiment, the cultivation is carried under aerobic conditions. In a further embodiment, the polyene polyketide generates a ¹H NMR spectrum essentially as shown in FIG. 3. In a further embodiment, the polyene polyketide generates an ¹H NMR spectrum essentially as shown in FIG. 10.

The invention further provides a method of inhibiting fungal cell growth, the method comprising contacting a fungal cell with a compound of Formula I or II, or a derivatives or salt thereof, such that the growth of the fungal cell is inhibited. In one embodiment, compound is part of a pharmaceutical composition comprising a compound of Formula I or II or a derivative or a salt thereof, together with a pharmaceutically acceptable carrier. In another embodiment, the compound is Compound 1, Compound 2 or a salt of Compound 1 or Compound 2. The invention further provides a method of inhibiting a fungal cell growth or infection in a mammal, the method comprising administering a therapeutically effective amount of a compound of Formula I or II, or a salt thereof, to a mammal having such a fungal cell growth or infection such that the fungal cell growth or infection is inhibited in the mammal. In one embodiment, the compound is part of a pharmaceutical composition comprising a compound of Formula I or II, or a salt thereof, together with a pharmaceutically acceptable carrier. In another embodiment, the pharmaceutical composition is Compound 1 or Compound 2, together with a pharmaceutically acceptable carrier.

The invention provides a compound of Formula I or II, or a salt thereof, for use as an antifungal agent. In one embodiment, the compound is Compound 1 or Compound 2, or a salt of Compound 1 or Compound 2, for use as an antifungal agent. The invention also provides a composition comprising a compound of Formula I or II for use as an antifungal agent. In one embodiment, the composition for use as an antifungal agent is Compound 1 or Compound 2, together with a pharmaceutically acceptable carrier.

The methods of the invention are useful for treating fungal infections or inhibiting the growth of fungal cells in mammals caused by Candida albicans. The invention also encompasses methods for treating or inhibiting other types of fungal infections in a subject, wherein said fungal infections include those caused by Candida sp. such as C. glabrata, C. lusitaniae C. parapsilosis, C. krusei, C. tropicalis, S. cerevisiae; Aspergillus sp. such as A. fumigatus, A. niger, A. terreus, A. flavus; Fusarium spp.; Scedosporium spp.; Cryptococcus spp.; Mucor ssp.; Histoplasma spp.; Trichosporon spp.; and Blastomyces spp. Such methods comprise administering to a subject suffering from the fungal infection, a therapeutically effective amount of a compound of Formula I or II, Compound 1 or Compound 2, or a pharmaceutically acceptable salt thereof. In one embodiment, the invention provides use of Compound 1 or Compound 2 as a fungal agent or in the treatment of fungal infection. Another embodiment, provides use of Compound 1 or Compound 2 in the preparation of a medicament for use in the treatment of fungal invention.

The invention also provides methods of inhibiting cancer cell growth, which comprise contacting said cancer cell with a compound of Formula I, Compound 1 or Compound 2, or a pharmaceutically acceptable salt thereof. The invention further encompasses methods for treating cancer in a subject, comprising administering to said subject suffering from said cancer, a therapeutically effective amount of a compound of Formula I, Formula II, Compound 1 or Compound 2 or a pharmaceutically acceptable salt thereof. Examples of cancers that may be treated or inhibited according to the methods of the invention include leukemia, non-small cell lung cancer, colon cancer, CNS cancer, melanoma, ovarian cancer, renal cancer, prostate cancer and breast cancer. One embodiment of the invention provides use of Compound 1 or Compound 2 as an anti-tumor agent. Another embodiment provides use of Compound 1 or compound 2 in the preparation of an anti-tumor medicament.

### BRIEF DESCRIPTION OF THE DRAWINGS

FIG. 1 is a mass spectrum for compound 1.

FIG. 2 is an ultraviolet spectrum for Compound 1.

FIG. 3 is a ¹H NMR spectrum for Compound 1.

FIG. 4 is a ¹³C NMR spectrum for Compound 1.

FIG. 5 is a ¹H—¹H gDQCOSY pulse sequence for Compound 1.

FIG. 6 is a ¹H—¹³C gHSQC pulse sequence for Compound 1.

FIG. 7 is a ¹H—¹³C gHMBC pulse sequence for Compound 1.

FIG. 8 is a mass spectrum for compound 2.

FIG. 9 is an ultraviolet spectrum for Compound 2.

FIG. 10 is a ¹H NMR spectrum for Compound 2.

FIG. 11 is a ¹³C NMR spectrum for Compound 2.

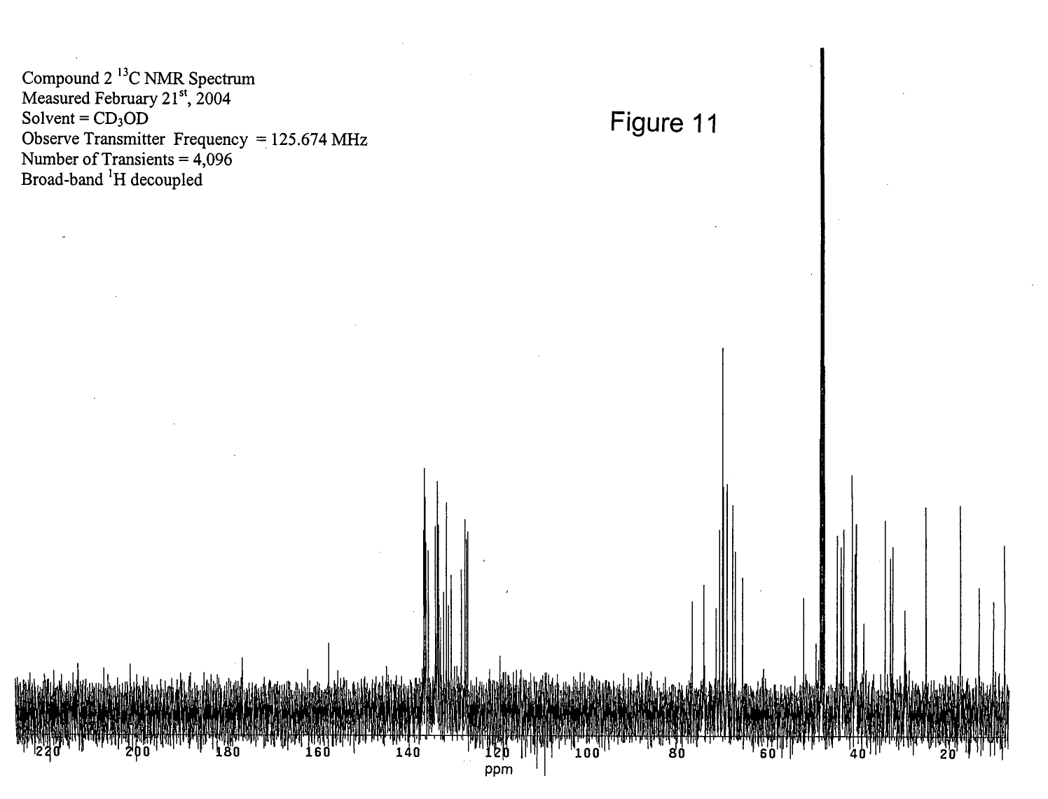

FIG. 12 is a ¹H—¹H gCOSY pulse sequence for Compound 2.

FIG. 13 is a ¹H—¹³C gHSQC pulse sequence for Compound 2.

FIG. 14 is a ¹H—¹³C gHMBC pulse sequence for Compound 2.

FIGS. 15a and 15b show ¹H NMR and ¹³C NMR assignments for Compound 1.

FIGS. 16a and 16b show ¹H NMR and ¹³C NMR assignments for Compound 2.

### DETAILED DESCRIPTION

The present invention relates to novel polyketide compounds, referred to herein as Compound 1 and Compound 2, which were isolated from strains of actinomycetes, Streptomyces sp. The invention further relates to pharmaceutically acceptable salts and derivatives of Compound 1 and Compound 2, and to methods for obtaining such compounds. One method of obtaining the compounds is by cultivating Streptomyces sp. strain NRRL B-12234, or a mutant or a variant thereof, under suitable Streptomyces sp. culture conditions, preferably using the fermentation protocol described herein.

The present invention also relates to pharmaceutical compositions comprising Compound 1 and Compound 2, and its pharmaceutically acceptable salts and derivatives. Compound 1 and Compound 2 are each useful as pharmaceuticals, for use as an inhibitor of fungal cell growth or for use as cytotoxic agents.

The following detailed description discloses how to make and use Compound 1 and Compound 2, and compositions containing these compounds to inhibit fungal growth and/or specific disease mediators.

Accordingly, certain aspects of the present invention relate to pharmaceutical compositions comprising the polyene polyketides of the invention together with a pharmaceutically acceptable carrier, methods of using the compositions to inhibit fungal growth, and methods of using the pharmaceutical compositions to treat diseases, including fungal infection.

I. Definitions

Certain terms, when used in this application, have their common meaning unless otherwise specified. For convenience, the meaning of some terms and phrases used in the specification, examples, and appended claims, are provided below.

The term alkyl refers to linear, branched or cyclic hydrocarbon groups. Examples of alkyl groups include, without limitation, methyl, ethyl, n-propyl, isopropyl, n-butyl, pentyl, hexyl, heptyl, cyclopentyl, cyclohexyl, cyclohexymethyl, and the like. Alkyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl, oxo, guanidino and formyl.

The term alkenyl refers to linear, branched or cyclic hydrocarbon groups containing at least one carbon-carbon double bond. Examples of alkenyl groups include, without limitation, vinyl, 1-propene-2-yl, 1-butene-4-yl, 2-butene-4-yl, 1-pentene-5-yl and the like. Alkenyl groups may optionally be substituted with substituents selected from acyl, amino, acylamino, acylbxy, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl, formyl, oxo and guanidino. The double bond portion(s) of the unsaturated hydrocarbon chain may be either in the cis or trans configuration.

The term cycloalkyl or cycloalkyl ring refers to a saturated or partially unsaturated carbocyclic ring in a single or fused carbocyclic ring system having from three to fifteen ring members. Examples of cycloalkyl groups include, without limitation, cyclopropyl, cyclobutyl, cyclopentyl, cyclohexyl, and cycloheptyl. Cycloalkyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl.

The term heterocyclyl, heterocyclic or heterocyclyl ring refers to a saturated or partially unsaturated ring containing one to four hetero atoms or hetero groups selected from O, N, NH, NR^{x}, PO₂, S, SO or SO₂ in a single or fused heterocyclic ring system having from three to fifteen ring members. Examples of a heterocyclyl, heterocyclic or heterocyclyl ring include, without limitation, morpholinyl, piperidinyl, and pyrrolidinyl. Heterocyclyl, heterocyclic or heterocyclyl ring may optionally be substituted with substituents selected from acyl, amino, acylamino, acyloxy, oxo, thiocarbonyl, imino, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl.

The term amino acid refers to a natural amino acid, a synthetic amino acid or a synthetic derivative of a natural amino acid. Examples of natural amino acids include, but are not limited to alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

The term halo is defined as a bromine, chlorine, fluorine or iodine.

The term aryl or aryl ring refers to aromatic groups in a single or fused ring system, having from five to fifteen ring members. Examples of aryl include, without limitation, phenyl, naphthyl, biphenyl, terphenyl. Aryl may optionally be substituted with one or more substituent group selected from acyl, amino, acylamino, acyloxy, azido, alkythio, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl.

The term heteroaryl or heteroaryl ring refers to aromatic groups in a single or fused ring system, having from five to fifteen ring members and containing at least one hetero atom such as O, N, S, SO and SO₂. Examples of heteroaryl groups include, without limitation, pyridinyl, thiazolyl, thiadiazoyl, isoquinolinyl, pyrazolyl, oxazolyl, oxadiazoyl, triazolyl, and pyrrolyl groups. Heteroaryl groups may opitionally be substituted with one or more substituent group selected from acyl, amino, acylamino, acyloxy, carboalkoxy, carboxy, carboxyamido, cyano, halo, hydroxyl, nitro, thio, thiocarbonyl, alkyl, alkenyl, alkynyl, cycloalkyl, heterocyclyl, aryl, heteroaryl, alkoxy, aryloxy, sulfinyl, sulfonyl, and formyl.

The terms “aralkyl” and “heteroaralkyl” refer to an aryl group or a heteroaryl group, respectively bonded directly through an alkyl group, such as benzyl. Aralkyl and heteroaralkyl may be optionally substituted as the aryl and heteroaryl groups.

Similarly, the terms “aralkenyl” and “heteroaralkenyl” refer to an aryl group or a heteroaryl group, respectively bonded directly through an alkene group, such as benzyl. Aralkenyl and heteroaralkenyl may be optionally substituted as the aryl and heteroaryl groups.

The compounds of the present invention can possess one or more asymetric carbon atoms and can exist as optical isomers forming mixtures of racemic or non-racemic compounds. The compounds of the present invention are useful as a single isomer or as a mixture of stereochemical isomeric forms. Diastereoisomers, i.e., nonsuperimposable stereochemical isomers, can be seperated by conventional means such as chromatography, distillation, crystallization or sublimation. The optical isomers can be obtained by resolution of the racemic mixtures according to conventional processes.

The invention encompasses isolated or purified compounds. An “isolated “purified” compound refers to a compound which represents at least 10%, 20%, 50%, 80% or 90% of the compound of the present invention present in a mixture, provided that the mixture comprising the compound of the invention has demonstrable (i.e. statistically significant) biological activity including antibacterial, antifungal and anticancer when tested in conventional biological assays known to a person skilled in the art.

As used herein, the term “treatment” refers to the application or administration of a therapeutic agent to a patient, or application or administration of a therapeutic agent to an isolated tissue or cell line from a patient, who has a disorder, e.g., a disease or condition, a symptom of disease, or a predisposition toward a disease, with the purpose to cure, heal,.alleviate, relieve, alter, remedy, ameliorate, improve, or affect the disease, the symptoms of disease, or the predisposition toward disease.

As used herein, a “pharmaceutical composition” comprises a pharmacologically effective amount of a polyketide compound and a pharmaceutically acceptable carrier. As used herein, “pharmacologically effective amount,” “therapeutically effective amount” or simply “effective amount” refers to that amount of a polyketide compound effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

The term “pharmaceutically acceptable salt” refers to both acid addition salts and base addition salts. The nature of the salt is not critical, provided that it is pharmaceutically acceptable. Exemplary acid addition salts include, without limitation, hydrochloric, hydrobromic, hydroiodic, nitric, carbonic, sulphuric, phosphoric, formic, acetic, citric, tartaric, succinic, oxalic, malic, glutamic, propionic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactaric, galacturonic acid and the like. Suitable pharmaceutically acceptable base addition salts include, without limitation, metallic salts made from aluminium, calcium, lithium, magnesium, potassium, sodium and zinc or organic salts made from N,N′-dibenzylethylenediamine, chloroprocaine, choline, diethanolamine, ethylenediamine, N-methylglucamine, lysine, procaine and the like. Additional examples of pharmaceutically acceptable salts are listed in Journal of Pharmaceutical Sciences (1977) 66:2. All of these salts may be prepared by conventional means from a polyketide compound of the present invention by treating the compound with the appropriate acid or base.

Unless otherwise indicated, all numbers expressing quantities of ingredients and properties such as molecular weight, reaction conditions, IC50 and so forth used in the specification and claims are to be understood as being modified in all instances by the term “about”. Accordingly, unless indicated to the contrary, the numerical parameters set forth in the present specification and attached claims are approximations. At the very least, and not as an attempt to limit the application of the doctrine of equivalents to the scope of the claims, each numerical parameter should at least be construed in light of the number of significant figures and by applying ordinary rounding techniques. Notwithstanding that the numerical ranges and parameters setting forth the broad scope of the invention are approximations, the numerical values set forth in the examples, tables and figures are reported as precisely as possible. Any numerical values may inherently contain certain errors resulting from variations in experiments, testing measurements, statistical analysis and such.

II. Polyketide Compounds of the Invention

The compounds of the present invention may be represented by Formula I:

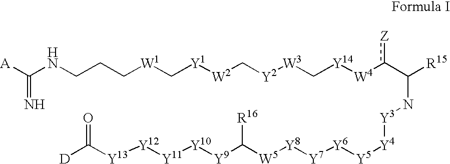

wherein,

A is selected from NHC(O)R¹, N═CR² R³, or NHR³, wherein R¹ R² and R³ is independently selected from the group consisting of H, C₁₋₆ alkyl, C₂₋₆ alkenyl, C₃₋₆ cycloalkyl, C₃₋₆ heterocycloalkyl, aryl and heteroaryl, wherein said alkyl, alkenyl, cycloalkyl, heterocycloalkyl, aryl and heteroaryl are optionally substituted with a group selected from halogen, oxo, OH, C₃₋₆alkyl, C₁₋₆ alkyl, NO₂, NH₂, cycloalkyl, heteroaryl or aryl, said C₃₋₆alkyl, C₁₋₆ alkyl, heteroaryl and aryl being optionally further substituted with one or more groups independently selected from halogen, OH, C₁₋₃ alkyl NO₂ or NH₂;

B is selected from

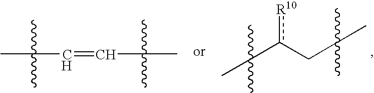

wherein R¹⁰ is OH, —OS(O)₂OH or, when the dotted line is a bond then R¹⁰ is oxo;

D is selected from OH or C₁₋₆alkoxy optionally substituted with 1 to 2 phenyl groups, wherein the phenyl group is optionally substituted with C₁₋₆ alkyl or halo;

W¹ and W² is each independently selected from

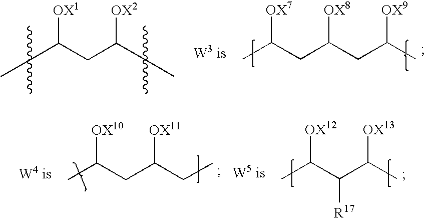

X¹, X², X⁷, X⁸, X⁹, X¹⁰, X¹¹, X¹² and X¹³ is each independently selected from H, —C(O)—R⁷ and a bond such that when any of two neighboring X¹, X², X⁷, X⁸, X⁹, X¹⁰, X¹¹, X¹² and X¹³ is a bond then the two neighboring oxygen atoms and their attached carbon atoms together form a six-membered acetal ring of the formula:

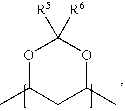

wherein R⁵, R⁶ and R⁷ is each independently selected from H, C₁₋₆ alkyl, C₂₋₇ alkenyl;

Y¹, Y², Y³, Y⁴, Y⁵, Y⁶, Y⁷, Y⁸, Y⁹, Y¹⁰, Y¹¹, Y¹², Y¹³ and Y¹⁴ is each independently selected from —CH₂—CH₂—, —CH═CH—,

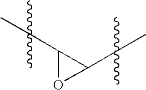

or —CH(OH)—CH(OH)—, wherein said —CH₂—CH₂—, —CH═CH— or —CH(OH)—CH(OH)— are optionally substituted with a methyl group;

Z is selected from OH, C₃₋₆ cycloalkyl, C₃₋₆ heterocycloalkyl, NHR⁸,

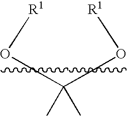

and when the dotted line is a bond then Z is oxo, or NC₁₋₆ alkyl, wherein R⁸ is selected from H, C₁₋₆ alkyl, C₂₋₆alkenyl or C₃₋₆cycloalkyl;

R¹⁵, R¹⁶ and R¹⁷ is each independently selected from H or CH₃; or a pharmaceutically acceptable salt thereof.

In another aspect, the invention provides compounds of Formula I wherein Z is oxo and all other groups are as previously defined; or a pharmaceutically acceptable salt thereof. In one embodiment, the invention provides compounds of Formula I, wherein Z is oxo, A is

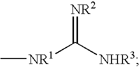

and all other groups are as previously defined; or a pharmaceutically acceptable salt thereof. In another embodiment, the invention provides compounds of Formula I, wherein Z is oxo, A is

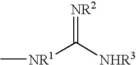

and B is

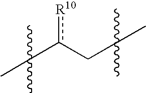

and all other groups are as previously defined; within this aspect R¹⁰ is —OS(O)₂OH. In an additional aspect of this embodiment R¹⁵, R¹⁶ and R¹⁷ are each independently CH₃. In a further aspect of this embodiment the invention provides compounds of Formula I, wherein Z is oxo, A is

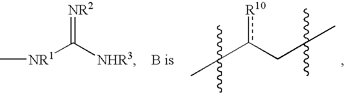

and all other groups are as previously defined; within this aspect R¹⁰ is OH. In an additional aspect of this embodiment R¹⁵, R¹⁶ and R¹⁷ are each independently.

The invention further provides compounds of Formula I, wherein D is OH; and all other groups are as previously defined; or a pharmaceutically acceptable salt thereof. In an aspect of this embodiment, the invention provides compounds of Formula I, wherein D is OH and Z is oxo; and all other groups are as previously defined; or a pharmaceutically acceptable salt thereof. In a further aspect of this embodiment, the invention provides compounds of Formula I wherein D is OH, Z is oxo and A is

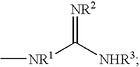

and all other groups are as previously defined; or a pharmaceutically acceptable salt thereof.

The invention further provides polyene polyketides of Formula II below:

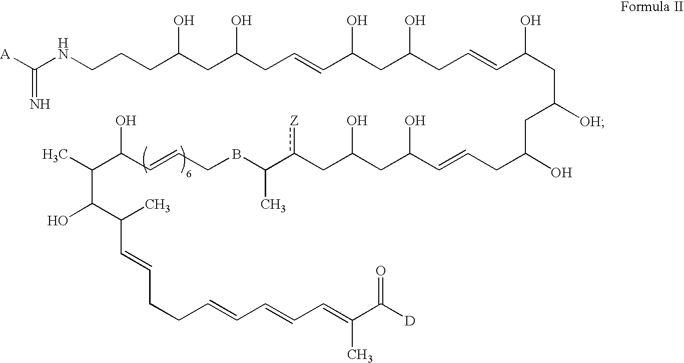

wherein A, B, D and Z are as described in any one of the embodiments above.

The following are exemplary compounds of the invention:

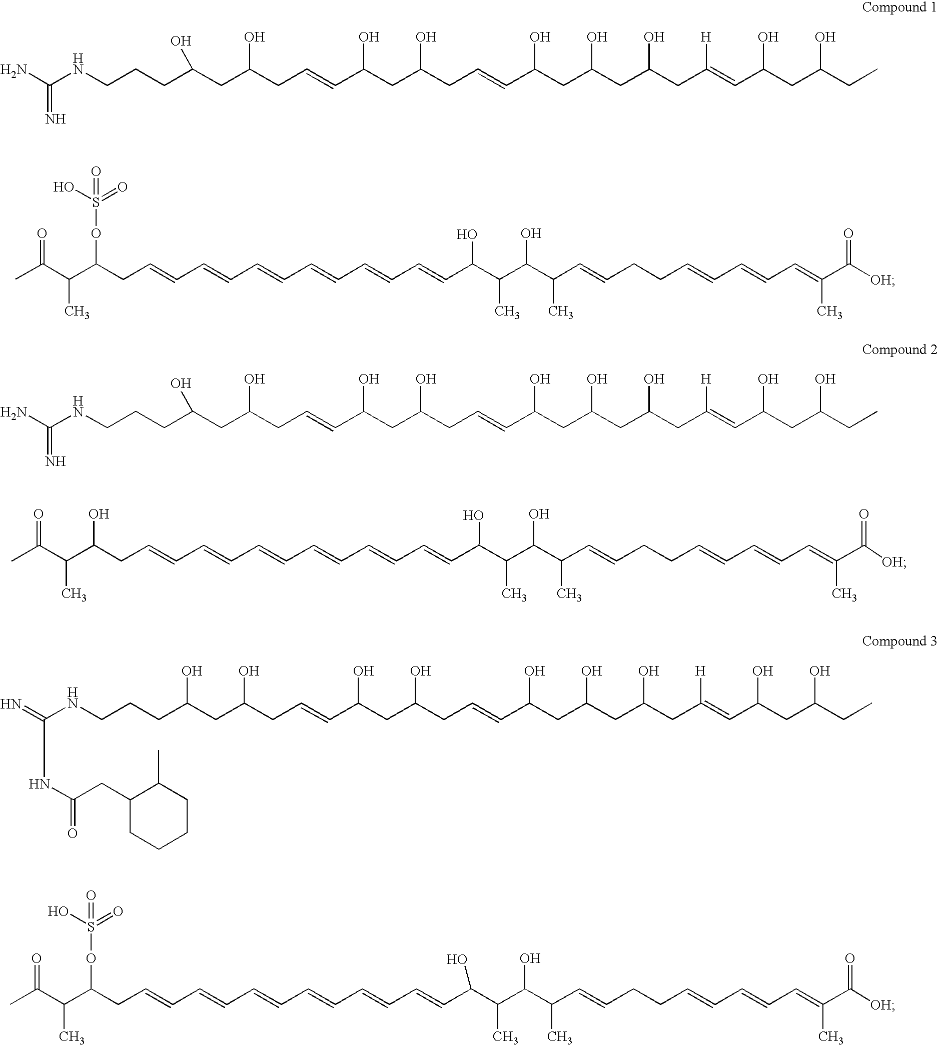

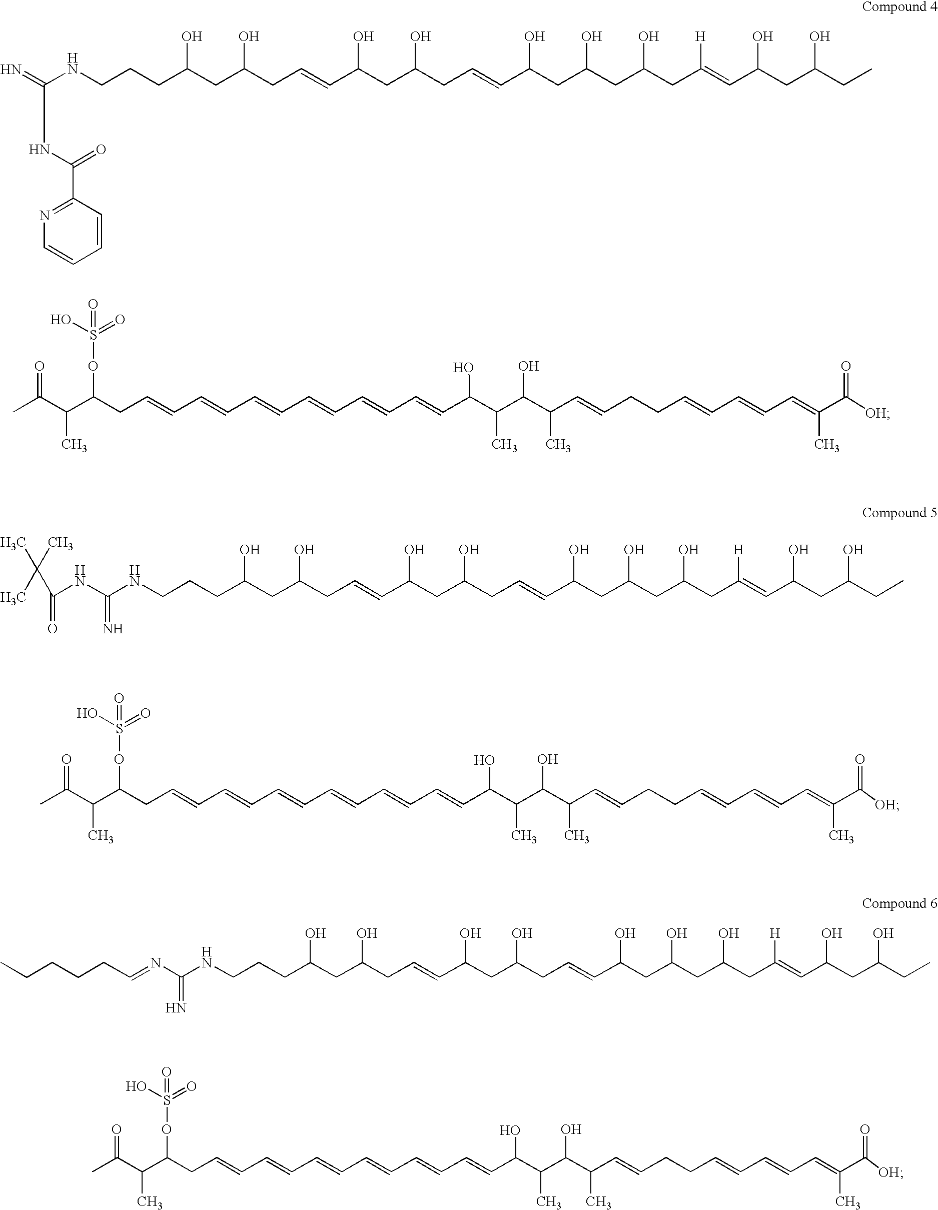

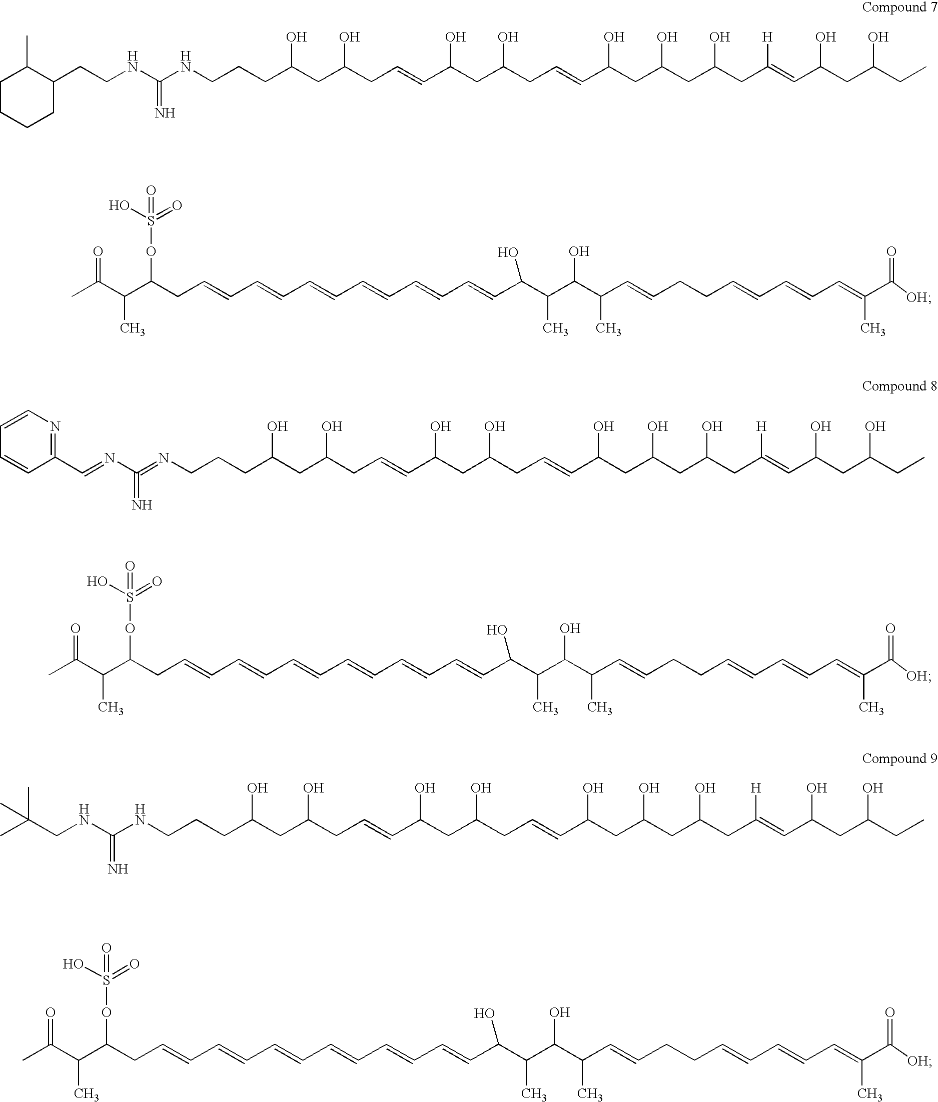

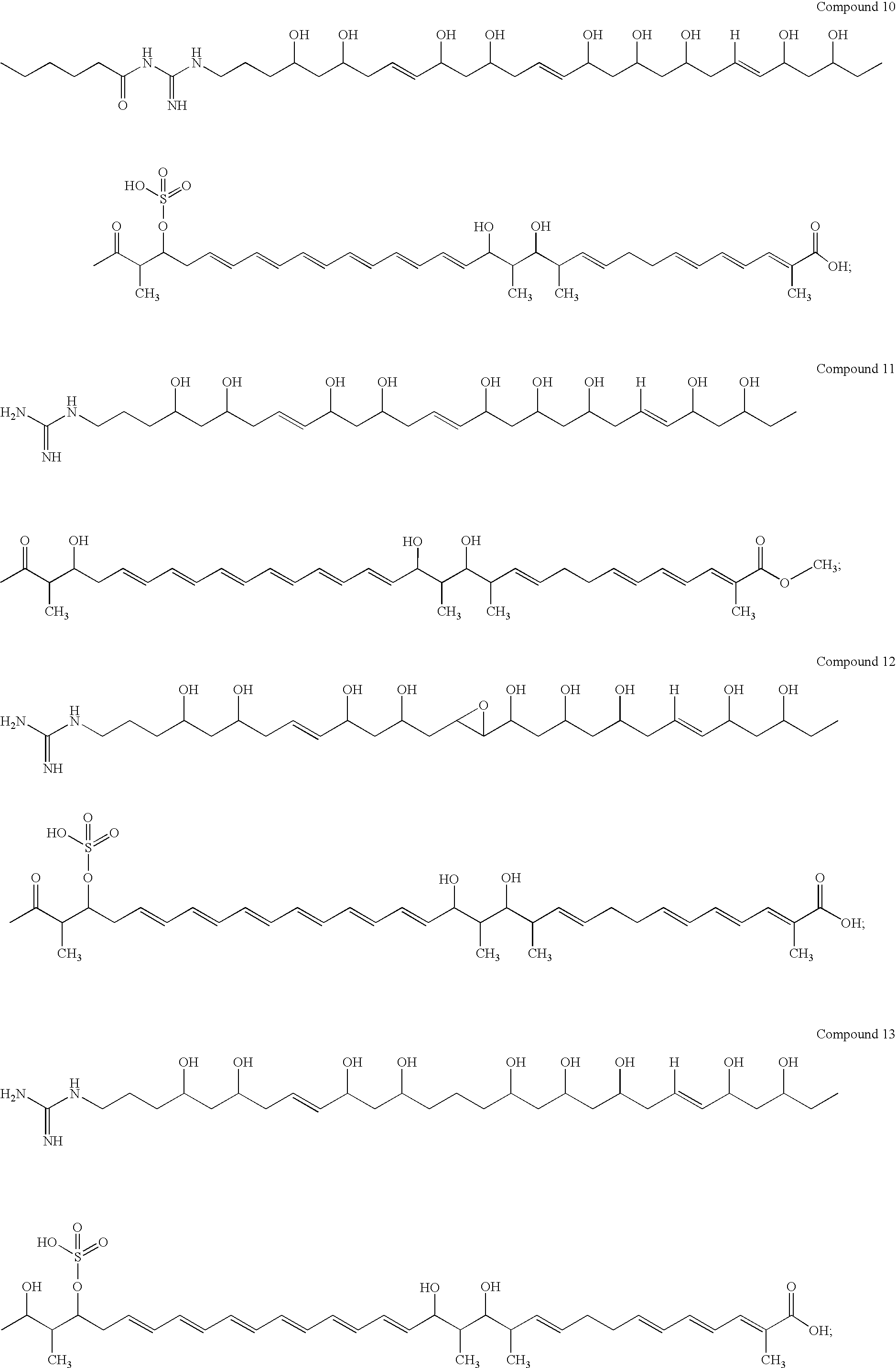

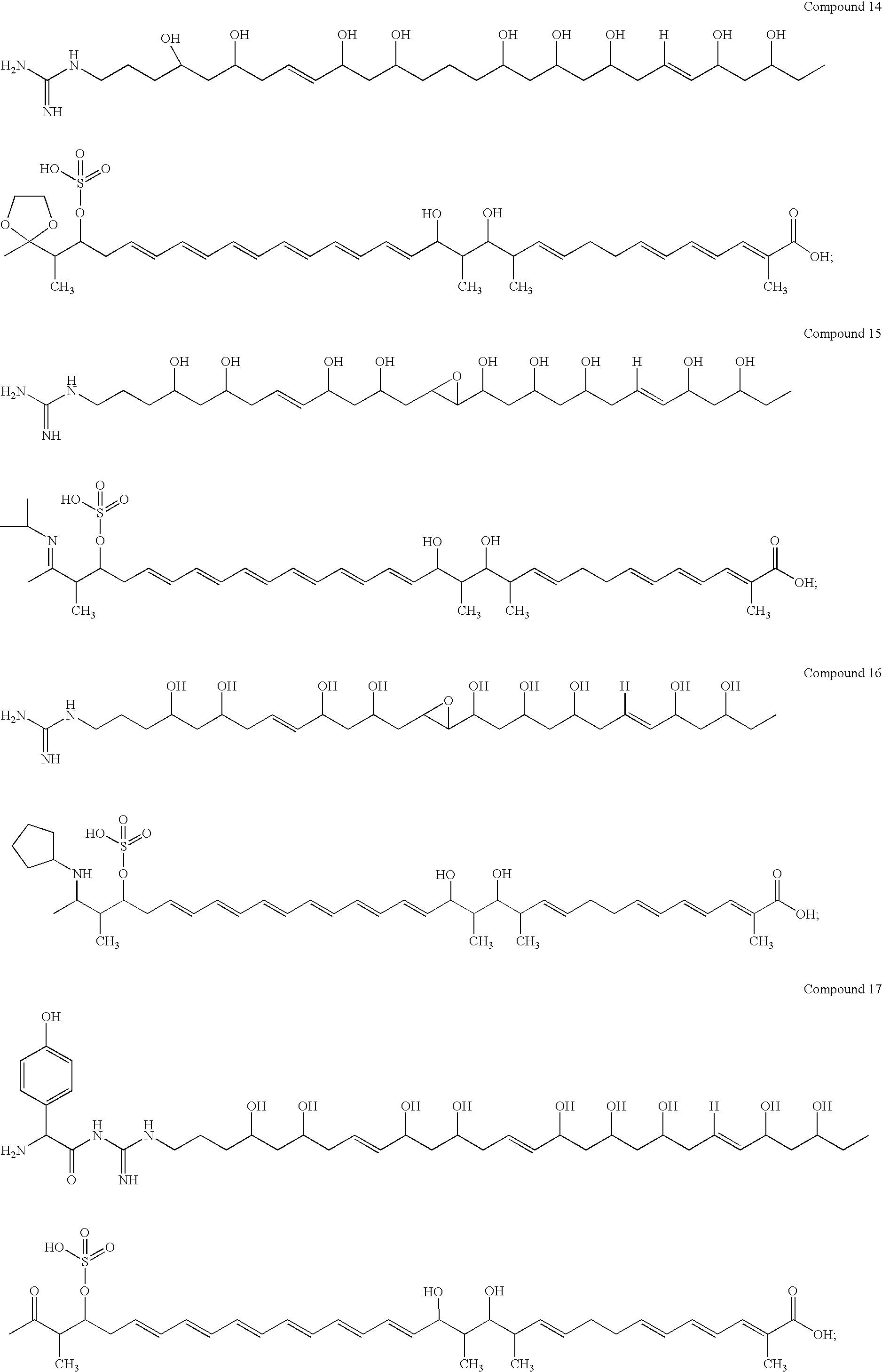

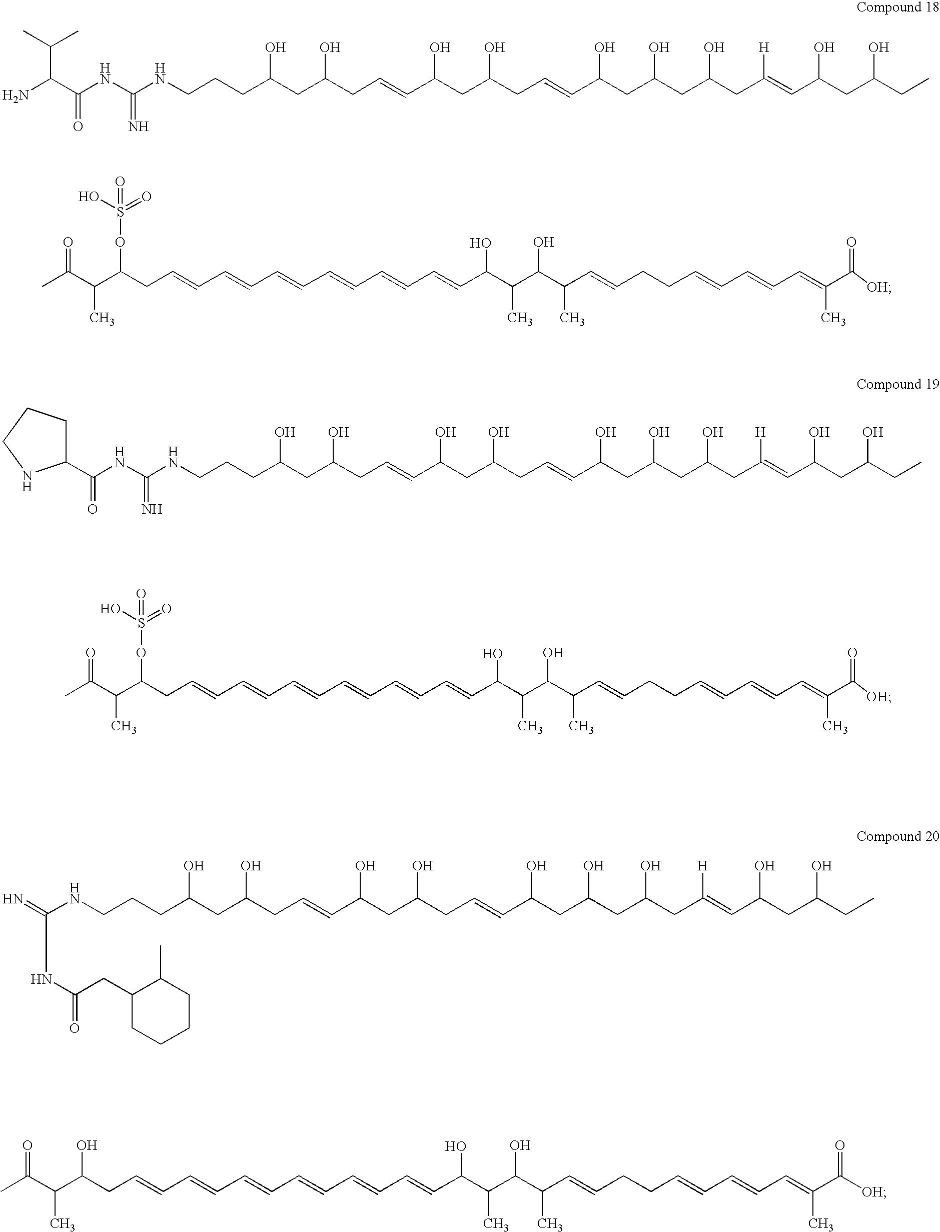

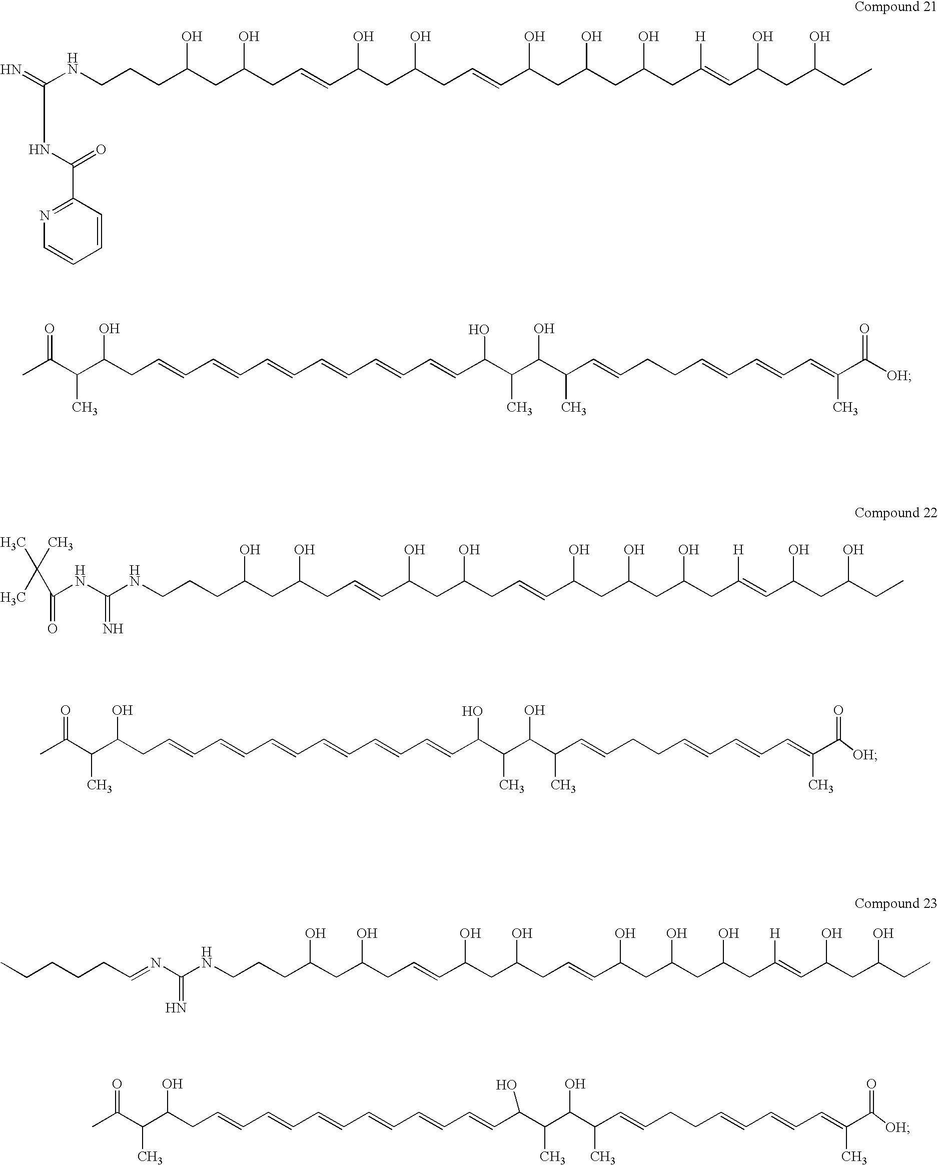

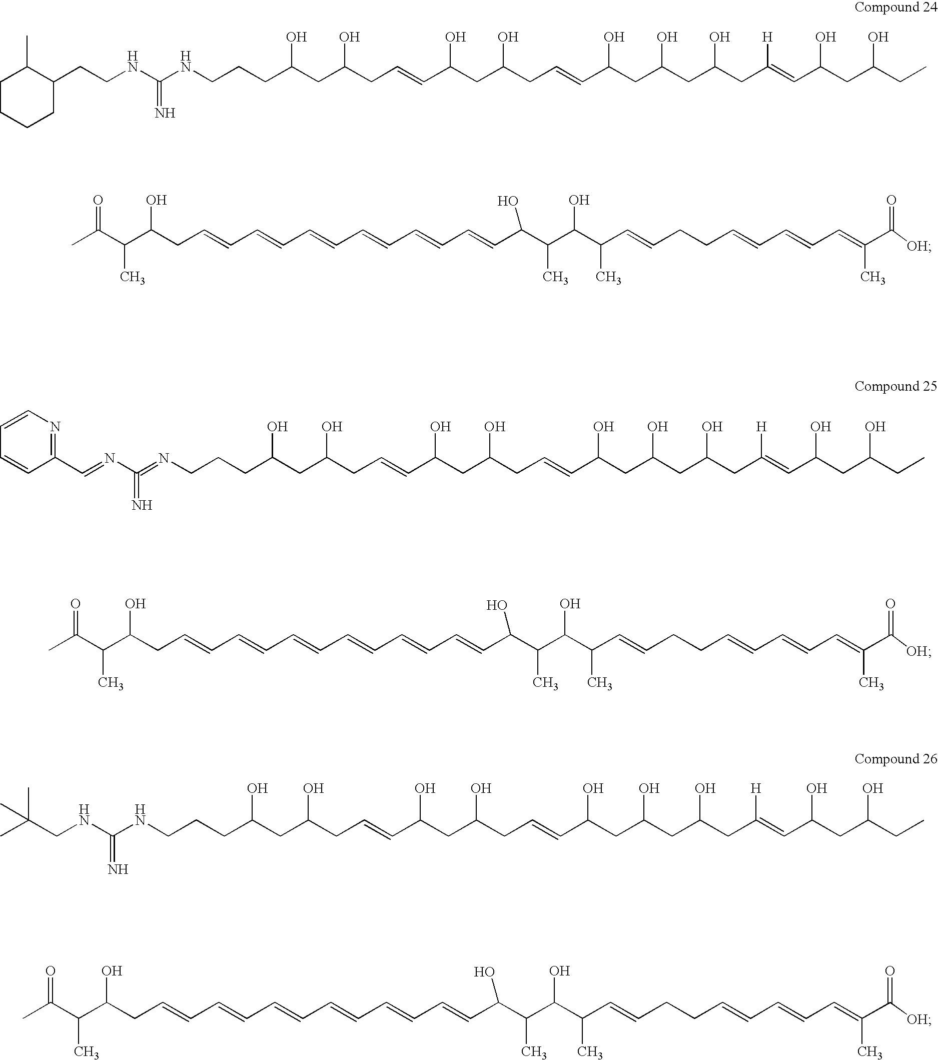

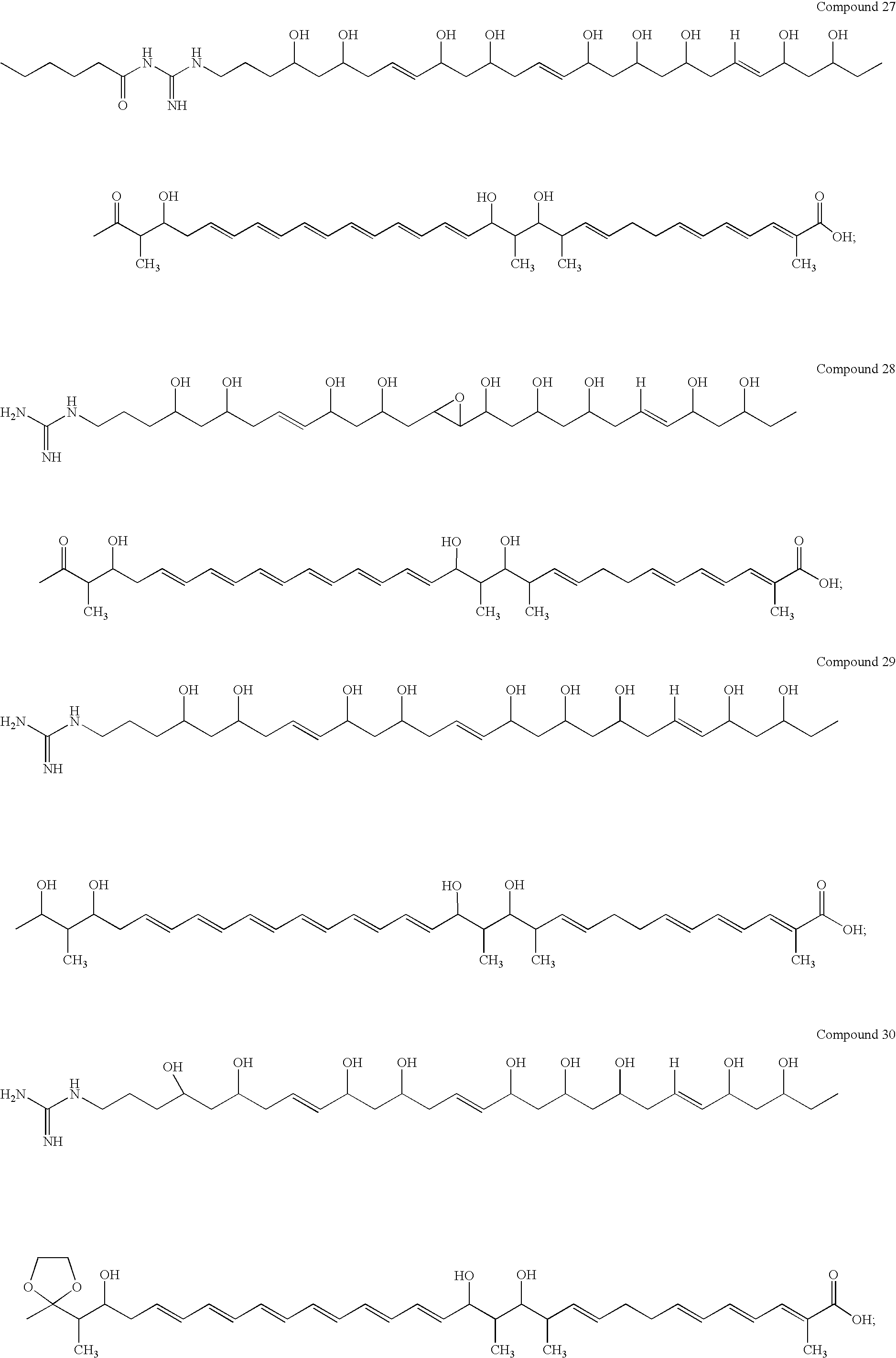

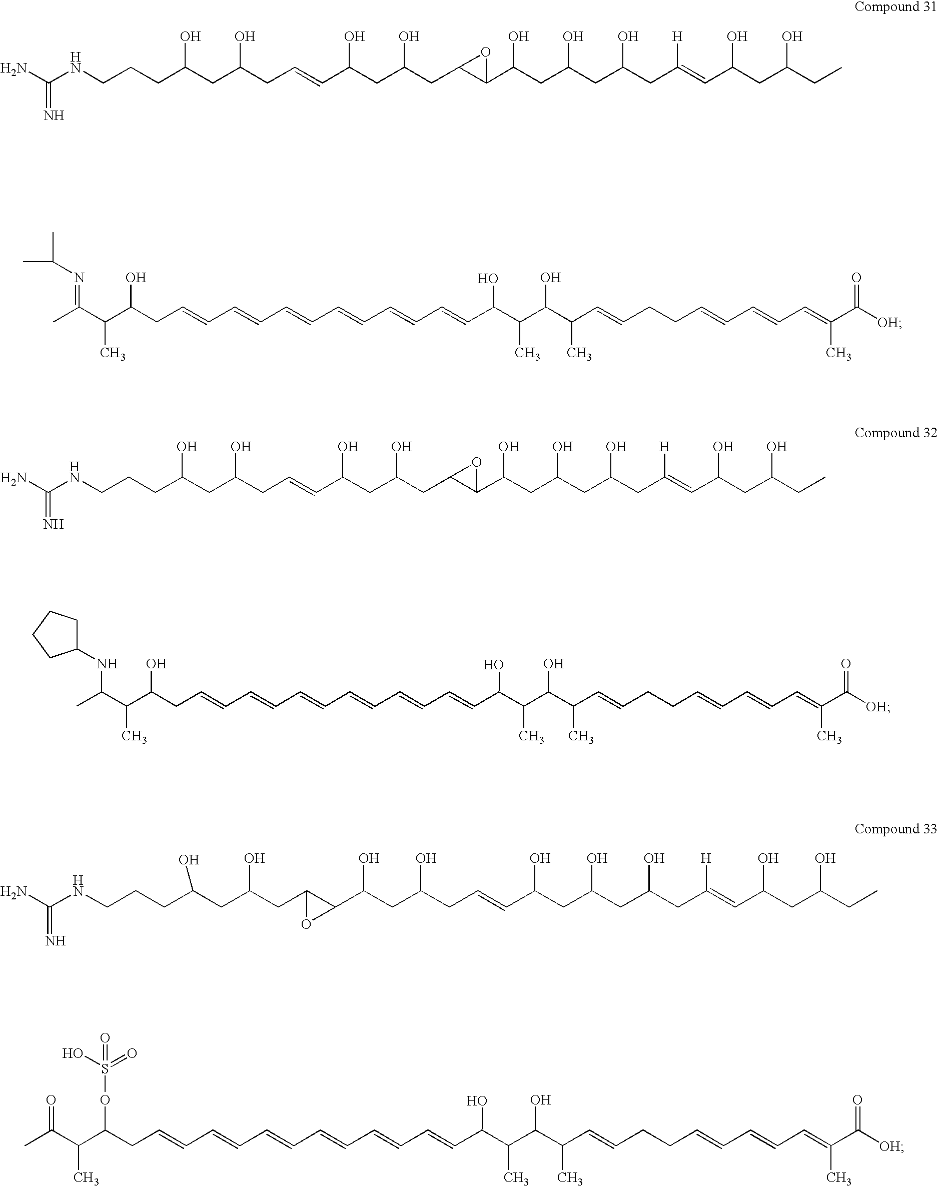

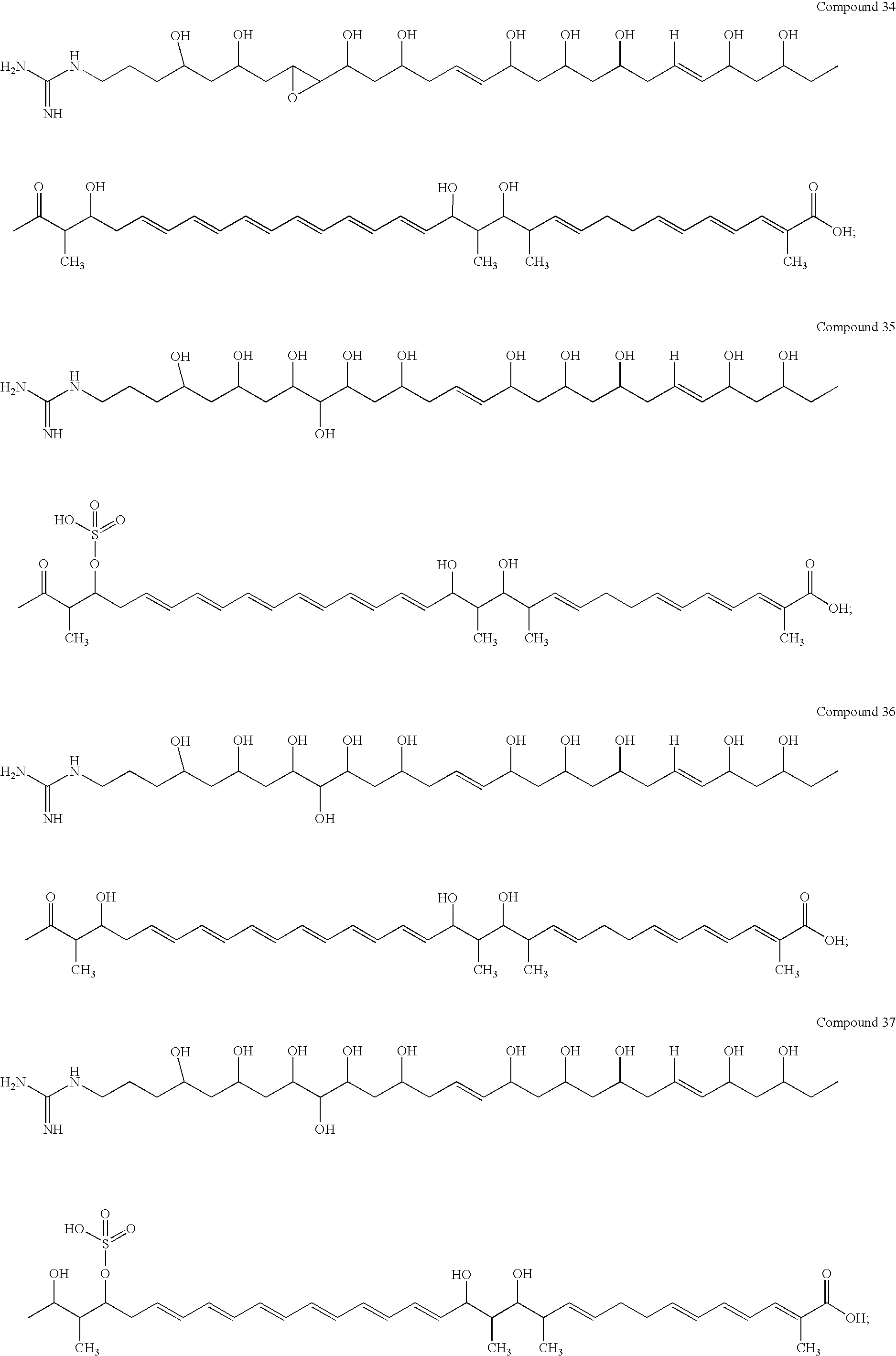

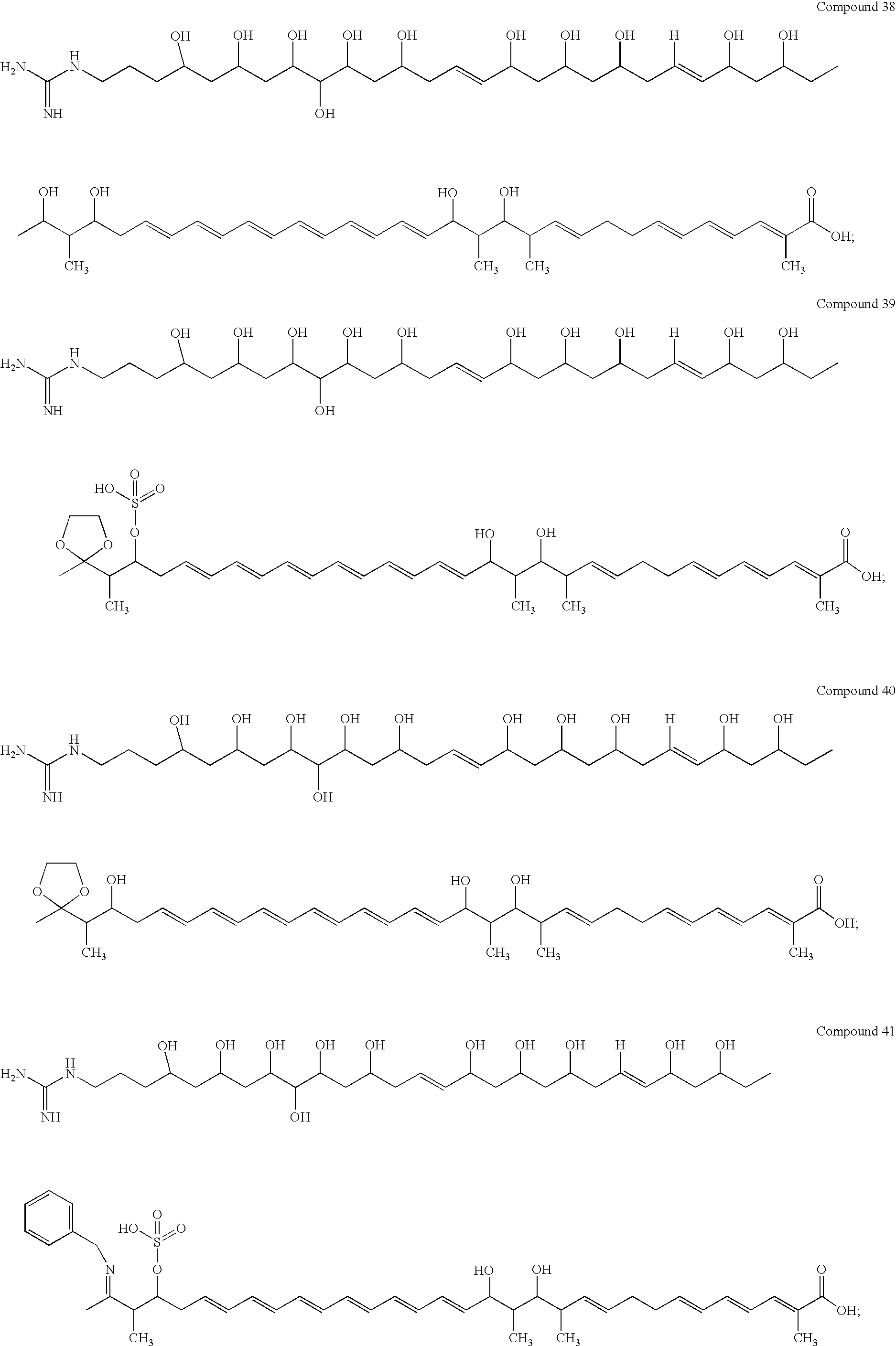

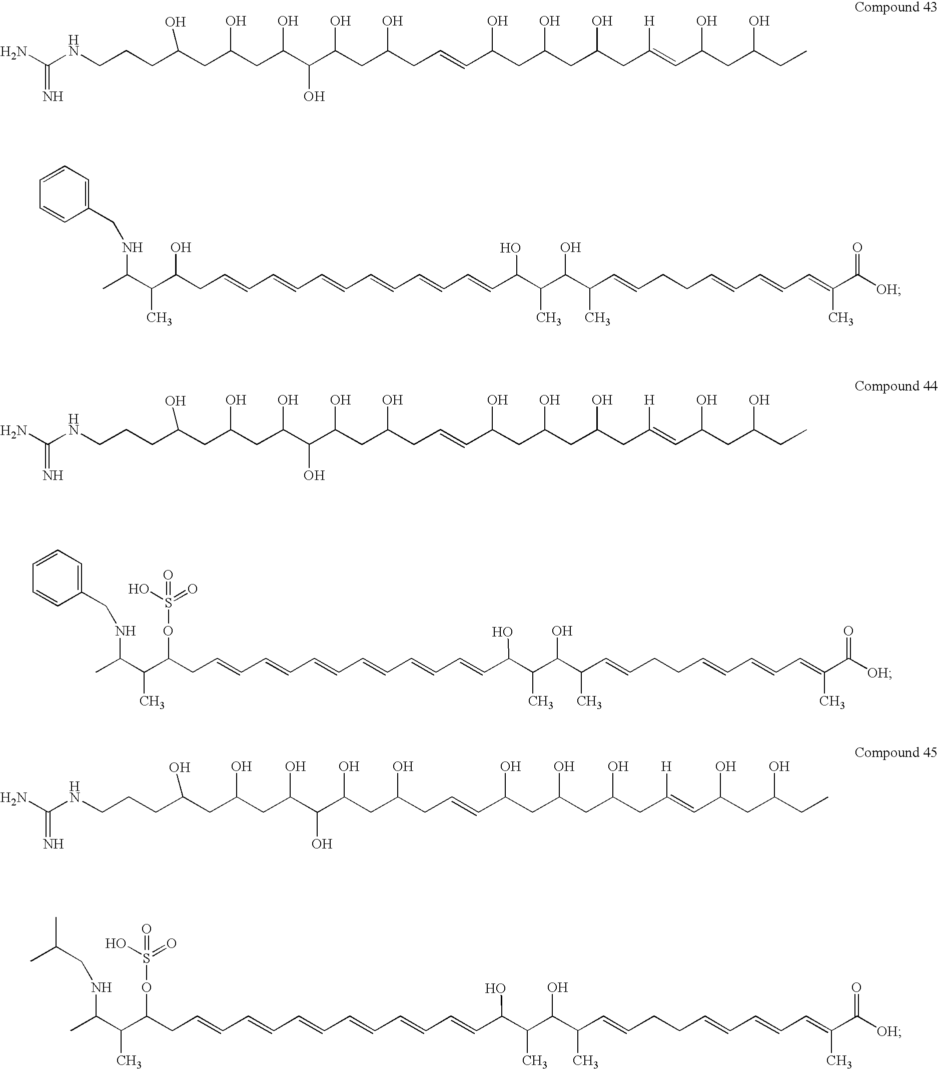

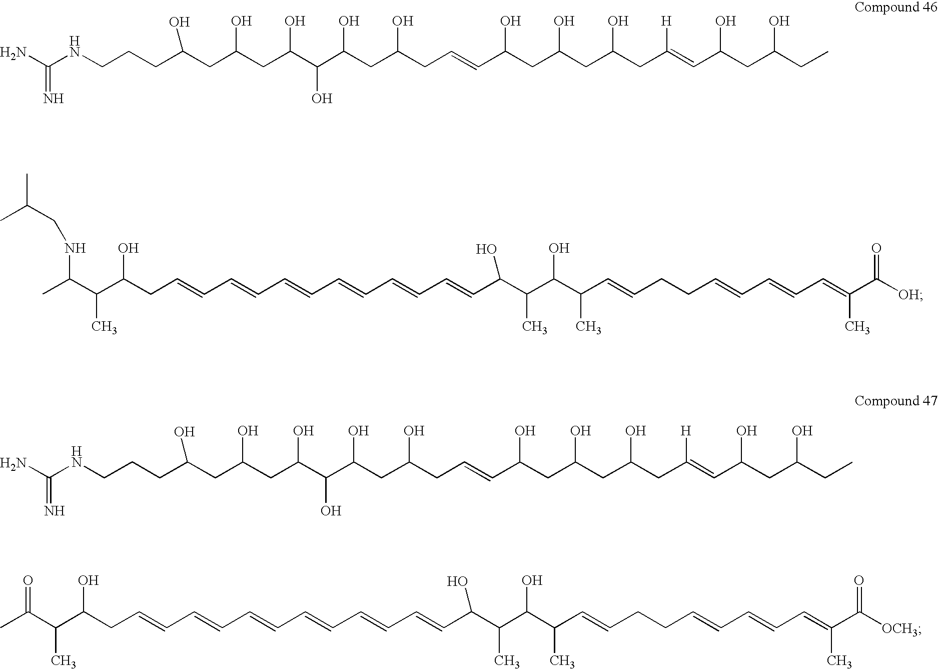

or a pharmaceutically acceptable salt of any one of Compound 1-47.

The compounds of this invention may be formulated into pharmaceutical compositions comprising a polyketide compound of Formula I or II in combination with a pharmaceutically acceptable carrier as discussed herein below.

In one aspect, the invention provides a composition comprising Compound 1 of the formula:

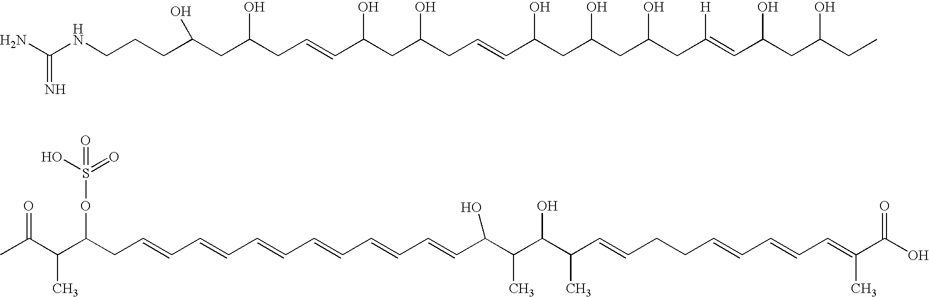

and a pharmaceutically acceptable carrier.

In another aspect, the invention provides a composition comprising Compound 2 of the formula:

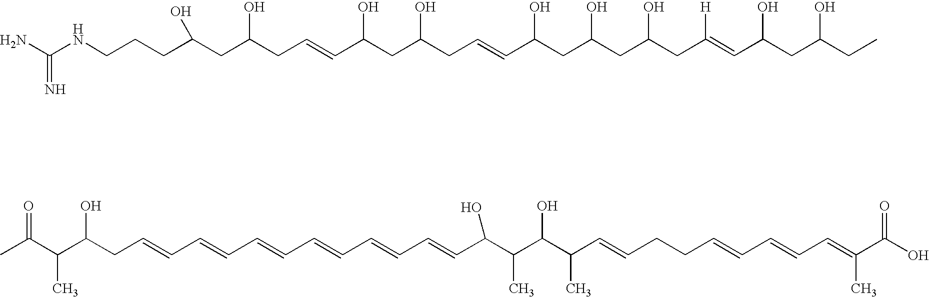

and a pharmaceutically acceptable carrier.

III. Method of Making Polene Polyketides by Fermentation

In one embodiment, Compound 1 and Compound 2 are obtained by cultivating a strain of Streptomyces sp., namely Streptomyces melanosporafaciens strain NRRL B-12234. Streptomyces melanosporafaciens strain NRRL B-12234 is available from the Agricultural Research Service Culture Collection, National Center for Agricultural Utilization Research, 1815 N. University Street, Peoria Ill. 61604, USA. However, it is to be understood that the present invention is not limited to use of the particular strain NRRL B-12234. Rather, the present invention contemplates the use of other organisms producing Compound 1 or Compound 2. Mutants or variants of NRRL B-12234 that can be derived from this organism by known means such as X-ray irradiation, ultraviolet irradiation, treatment with a chemical mutagen such as a nitrogen mustard, phage exposure, antibiotic resistance selection and the like.

The polyene polyketide compounds of the invention may be biosynthesized by various microorganisms. Microorganisms that may synthesize the polyene polyketides of the invention include but are not limited to bacteria of the order Actinomycetales, also referred to as actinomycetes. Non-limiting examples of members belonging to the genera of Actinomycetes include Nocardia, Geodermatophilus, Actinoplanes, Micromonospora, Nocardioides, Saccharothrix, Amycolatopsis, Kutzneria, Saccharomonospora, Saccharopolyspora, Kitasatospora, Streptomyces, Microbispora, Streptosporangium, and Actinomadura. The taxonomy of actinomycetes is complex and reference is made to Goodfellow, Suprageneric Classification of Actinomycetes (1989); Bergey's Manual of Systematic Bacteriology, Vol. 4 (Williams and Wilkins, Baltimore, pp. 2322-2339); and to Embley and Stackebrandt, “The molecular phylogeny and systematics of the actinomycetes,” Annu. Rev. Microbiol. (1994) 48:257-289, each is hereby incorporated by reference in its entirety, for genera that may synthesize the compounds of the invention.

An actinomycetes strain is selected and cultivated in culture medium containing known nutritional sources for actinomycetes, such media having assimilable sources of carbon, nitrogen, plus optional inorganic salts and other known growth factors at a pH of about 6 to about 9. Suitable media components include, but are not limited to, glucose, sucrose, mannitol, lactose, cane molasses, soluble starch, corn starch, corn dextrin, potato dextrin, linseed meal, corn steep solids, corn steep liquor, Distiller's Solubles™, dried yeast, yeast extract, malt extract, Pharmamedia™, glycerol, N-Z amine A, soybean powder, soybean flour, soybean meal, beef extract, meat extract, fish meal, Bacto-peptone, Bacto-tryptone, casamino acid, thiamine, L-glutamine, L-arginine, tomato paste, oatmeal, MgSO₄.7H₂O, MgSO₄, MgCl₂.6H₂O, CaCO₃, NaCl, Na acetate, KH₂PO₄, K₂HPO₄, K₂SO₄, Na₂HPO₄, FeSO₄.7H₂O, FeCl₂.4H₂O, ferric ammonium citrate, KI, Nal, (NH₄)₂SO₄, NH₄H₂PO₄, NH₄NO₃, K₂SO₄, ZnCl₂, ZnSO₄.7H₂O, ZnSO₄.5H₂O, MnCl₂.4H₂O, MnSO₄, CuSO₄.5H₂O, CoCl₂.2H₂O, phytic acid, casamino acid, proflo oil and morpholinopropanesulfonic acid (MOPS).

The culture media inoculated with a polyene polyketide producing microorganism may be aerated by incubating the inoculated culture media with agitation, for example, shaking on a rotary shaker, or a shaking water bath. Aeration may also be achieved by the injection of air, oxygen or an appropriate gaseous mixture to the inoculated culture media during incubation. Following cultivation, the polyene polyketide compound can be extracted and isolated from the cultivated culture media by techniques known to a person skilled in the art and/or disclosed herein, including for example centrifugation, chromatography, adsorption, filtration. For example, the cultivated culture media can be mixed with a suitable organic solvent such as n-butanol, n-butyl acetate or 4-methyl-2-pentanone, the organic layer can be separated for example, by centrifugation followed by the removal of the solvent, by evaporation to dryness or by evaporation to dryness under vacuum. The resulting residue can optionally be reconstituted with for example water, ethanol, ethyl acetate, methanol or a mixture thereof, and re-extracted with a suitable organic solvent such as hexane, carbon tetrachloride, methylene chloride or a mixture thereof. Following removal of the solvent, the compounds may be further purified by the use of standard techniques, such as chromatography.

The polyene polyketide compound biosynthesized by microorganisms may optionally be subjected to random and/or directed chemical modifications to form derivatives or structural analogs of Compound 1 or 2 covered by Formula I or Formula II, such derivatives or structural analogs having similar functional activities being within the scope of the present invention. Methods known in the art and described herein are use to produce polyene polyketide compounds of Formula I or Formula II by chemical modification of the polyene polyketides produced biosynthetically, for example Compound 1 and Compound 2.

VI. Production of Polyene Polyketides by Chemical Modification

Polyene polyketides of Formula I or Formula II are generated by biofermentation followed by standard organic chemical modification of the polyene polyketide produced. Preferred polyene polyketides for chemical modification include Compound 1 and Compound 2. General principles of organic chemistry required for making and manipulating the compounds of Formula I and II, including functional moieties, reactivity and common protocols are described, for example, in “Advanced Organic Chemistry”, 3^{rd} Edition by Jerry March (1985), which is incorporated herein by reference in its entirety. In addition, it will be appreciated by one of ordinary skill in the art that the synthetic methods described herein may use a variety of protecting groups, whether or not they are explicitly described. A “protecting group” as used herein means a moiety used to block one or more functional moieties such as reactive groups including oxygen, sulfur or nitrogen so that a reaction may be carried out selectively at another reactive site in a polyfunctional compound. General principles for the use of protective groups, their applicability to specific functional groups and their uses are described for example in T. H. Greene and P. G. M. Wuts, Protective Groups in Organic Synthesis, 3^{rd} Edition, John Wiley & Sons, New York (1999).

Those skilled in the art will readily appreciate that many synthetic chemical processes may be used to produce polyketides based on Compound 1 or Compound 2. The following schemes are exemplary of the routine chemical modifications that may be used to produce compounds of Formula I or II. Any chemical synthetic process known to a person skilled in the art providing the structures described herein may be used and are therefore comprised in the present invention.

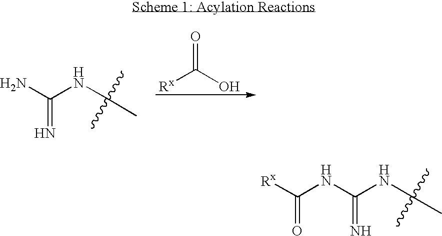

wherein R^{x} represents C₁₋₆ alkyl, C₂₋₆ alkene, aryl or heteroaryl; and

wherein R^{x}C(O)OH represents a naturally occurring amino acid (N-protected with a suitable protecting group such as N-benzyloxycarbonyl (CBZ), N-t-butoxycarbonyl (t-BOC), or N-fluoren-9-ylmethoxycarbonyl (FMOC).

### Scheme 1

In Scheme 1, carboxylic acid R^{x}C(O)OH is coupled to the guanidine residue using a coupling reagent such as EDC (1-(3-dimethylaminopropyl)-3-diisopropylethylcarbodiimide hydrochloride) in the presence of a base such as DIPEA (N,N-disopropylethylamine) in the appropriate solvent (for example: N,N-diimethylformamide). Scheme 1 is used to obtain Compounds 3, 4, 5 and 10 from Compound 1 and to obtain Compounds 20, 21, 22 and 27 from Compound 2.

Scheme 1 is followed by appropriate deprotection of the amino group, for example, hydrogen with catalytic Palladium on Charcoal to remove CBZ, trifluoroacetic acid to remove t-BOC group, or piperidine for the FMOC group, to obtain Compounds 17, 18 and 19 from Compound 1.

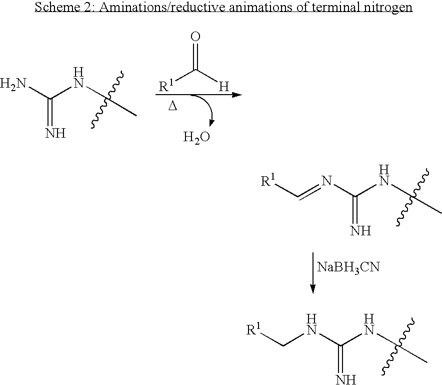

wherein R¹ is as previously defined.

### Scheme 2

In Scheme 2, an imine (schiff base) intermediate is obtained from the addition of the guanidine residue to an aldehyde R¹CHO. This intermediate is converted to the secondary amine by a reducing agent such as sodium cyanoborohydride (NaBH₃CN) in the appropriate solvent. Scheme 2 is used to obtain Compounds 6, 7, 8 and 9 from Compound 1, and to obtain Compounds 23, 24, 25 and 26 from Compound 2.

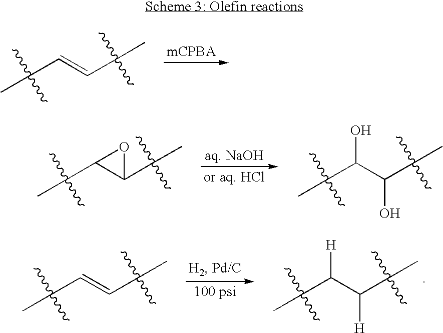

### Scheme 3

In Scheme 3, an epoxide is obtained from epoxidation of the olefin group by an oxidizing agent such as mCPBA (3-chloroperbenzoic acid). The epoxide is also opened in basic or acidic aqueous conditions to obtain the diol. A saturated alkyl is obtained from hydrogenation of olefin by palladium catalysis under hydrogen. Scheme 3 is used to obtain Compounds 12 and 33 from Compound 1 and Compounds 28 and 34 from Compound 2. Scheme 3 is further used to obtain Compound 35 from Compound 33 and Compound 36 from Compound 34.

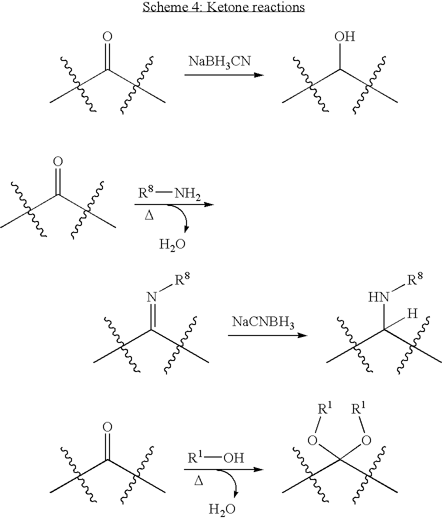

wherein R¹ and R⁸ are as previously defined.

### Scheme 4

In Scheme 4 a ketone moiety is reduced by a reducing agent such as NaBH₃CN to obtain the secondary alcohol; an amine compound is obtained by standard reductive amination of the ketone residue; and a ketal is obtained by the addition of alcohol or diol to the ketone in suitable acid catalysis conditions such as p-toluenesulfonic acid in boiling benzene using a Dean-Stark apparatus to remove the water formed. Scheme 4 is used to obtain Compounds 13 and 14 from Compound 1, and Compounds 15 and 16 from Compound 12. Scheme 4 is further used to obtain Compounds 29 and 30 from Compound 2, and Compounds 31 and 32 from Compound 28. Scheme 4 is further used to obtain Compounds 39, 41, 43 and 45 from Compound 35, and Compounds 40, 42, 44 and 46 from Compound 36.

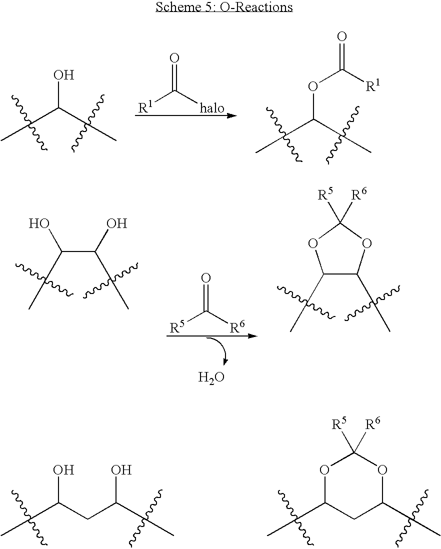

wherein R¹, R⁵ and R⁶ are as previously defined.

### Scheme 5

In Scheme 5 an alcohol is esterified by standard procedures like addition of R¹C(O)halo (halo is a suitable leaving group such as Cl and Br) in the presence of a base, and a 5 or 6 member cyclic ketal or acetal is obtained from the reaction of a diol with the appropriate ketone or aldehyde under acid catalysis with removal of the water produced.

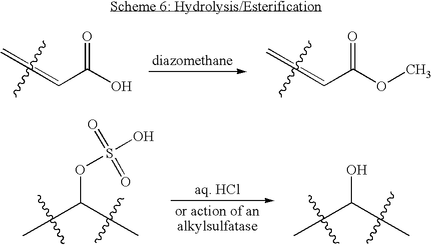

### Scheme 6

In Scheme 6 a carboxylic ester is obtained by standard esterification of terminal carboxylic acid (for example diazomethane in dry THF under argon), and a secondary alcohol is obtained by hydrolysis of the sulfate residue in aqueous acidic conditions. Scheme 6 is used to obtain Compound 11 from Compound 2 and Compound 47 from Compound 36, and to obtain Compound 2 from Compound 1.

VII. Pharmaceutical Compositions Comprising Polyene Polyketides

In another embodiment, the invention relates to pharmaceutical compositions comprising a polyene polyketide, as described in the preceding section, and a pharmaceutically acceptable carrier as described below. The pharmaceutical composition comprising the polyene polyketide is useful for treating a variety of diseases and disorders, including antifungal infections and tumor growth.

The compounds of the present invention, or pharmaceutically acceptable salts thereof, can be formulated for oral, intravenous, intramuscular, subcutaneous, intraocular, topical or parenteral administration for the therapeutic or prophylactic treatment of diseases, particularly bacterial and fungal infections. For oral or parental administration, compounds of the present invention can be mixed with conventional pharmaceutical carriers and excipients and used in the form of tablets, capsules, elixirs, suspensions, syrups, wafers and the like. The compositions comprising a compound of this present invention will contain from about 0.1% to about 99.9%, about 5% to about 95%, about 10% to about 80% or about 15% to about 60% by weight of the active compound.

The pharmaceutical preparations disclosed herein are prepared in accordance with standard procedures and are administered at dosages that are selected to reduce, prevent, or eliminate fungal infection or tumor growth (See, e.g., Remington's Pharmaceutical Sciences, Mack Publishing Company, Easton, Pa. and Goodman and Gilman's the Pharmaceutical Basis of Therapeutics, Pergamon Press, N.Y., N.Y., the contents of which are incorporated herein by reference, for a general description of the methods for administering various antimicrobial agents for human therapy). The compositions of the present invention can be delivered using controlled (e.g., capsules) or sustained release delivery systems (e.g., bioerodable matrices). Exemplary delayed release delivery systems for drug delivery that are suitable for administration of the compositions of the invention (preferably of Formula I) are described in U.S. Pat. No. 4,452,775 (issued to Kent), U.S. Pat. No. 5,239,660 (issued to Leonard), U.S. Pat. No. 3,854,480 (issued to Zaffaroni).

The pharmaceutically-acceptable compositions of the present invention comprise one or more compounds of the present invention in association with one or more non-toxic, pharmaceutically-acceptable carriers and/or diluents and/or adjuvants and/or excipients, collectively referred to herein as “carrier” materials, and if desired other active ingredients. The compositions may contain common carriers and excipients, such as corn starch or gelatin, lactose, sucrose, microcrystalline cellulose, kaolin, mannitol, dicalcium phosphate, sodium chloride and alginic acid. The compositions may contain crosarmellose sodium, microcrystalline cellulose, sodium starch glycolate and alginic acid.

Tablet binders that can be included are acacia, methylcellulose, sodium carboxymethylcellulose, polyvinylpyrrolidone (Providone), hydroxypropyl methylcellulose, sucrose, starch and ethylcellulose.

Lubricants that can be used include magnesium stearate or other metallic stearates, stearic acid, silicon fluid, talc, waxes, oils and colloical silica.

Flavoring agents such as peppermint, oil of wintergreen, cherry flavoring or the like can also be used. It may also be desirable to add a coloring agent to make the dosage form more aesthetic in appearance or to help identify the product comprising a compound of the present invention.

For oral use, solid formulations such as tablets and capsules are particularly useful. Sustained released or enterically coated preparations may also be devised. For pediatric and geriatric applications, suspension, syrups and chewable tablets are especially suitable. For oral administration, the pharmaceutical compositions are in the form of, for example, a tablet, capsule, suspension or liquid. The pharmaceutical composition is preferably made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica or talc: disintegrants, for example, potato starch, flavoring or coloring agents, or acceptable wetting agents. Oral liquid preparations generally are in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For intravenous (IV) use, compounds of the present invention can be dissolved or suspended in any of the commonly used intravenous fluids and administered by infusion. Intravenous fluids include, without limitation, physiological saline or Ringer's™ solution.

Formulations for parental administration can be in the form of aqueous or non-aqueous isotonic sterile injection solutions or suspensions. These solutions or suspensions can be prepared from sterile powders or granules having one or more of the carriers mentioned for use in the formulations for oral administration. The compounds can be dissolved in polyethylene glycol, propylene glycol, ethanol, corn oil, benzyl alcohol, sodium chloride, and/or various buffers.

For intramuscular preparations, a sterile formulation of compounds of the present invention or suitable soluble salts forming the compound, can be dissolved and administered in a pharmaceutical diluent such as Water-for-Injection (WFI), physiological saline or 5% glucose. A suitable insoluble form of the compound may be prepared and administered as a suspension in an aqueous base or a pharmaceutically acceptable oil base, e.g. an ester of a long chain fatty acid such as ethyl oleate.

For topical use the compounds of present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient.

For application to the eyes or ears, the compounds of the present invention can be presented in liquid or semi-liquid form formulated in hydrophobic or hydrophilic bases as ointments, creams, lotions, paints or powders.

For rectal administration the compounds of the present invention can be administered in the form of suppositories admixed with conventional carriers such as cocoa butter, wax or other glyceride.

Alternatively, the compound of the present invention can be in powder form for reconstitution in the appropriate pharmaceutically acceptable carrier at the time of delivery. In another embodiment, the unit dosage form of the compound can be a solution of the compound or a salt thereof in a suitable diluent in sterile, hermetically sealed ampoules.

The amount of the compound of the present invention in a unit dosage comprises a therapeutically-effective amount of at least one active compound of the present invention which may vary depending on the recipient subject, route and frequency of administration. A recipient subject refers to a plant, a cell culture or an animal such as an ovine or a mammal including a human.

According to this aspect of the present invention, the novel compositions disclosed herein are placed in a pharmaceutically acceptable carrier and are delivered to a recipient subject (including a human subject) in accordance with known methods of drug delivery. In general, the methods of the invention for delivering the compositions of the invention in vivo utilize art-recognized protocols for delivering the agent with the only substantial procedural modification being the substitution of the compounds of the present invention for the drugs in the art-recognized protocols.

Likewise, the methods for using the claimed composition for treating cells in culture, for example, to eliminate or reduce the level of fungal contamination of a cell culture, utilize art-recognized protocols for treating cell cultures with antibacterial or antifungal agent(s) with the only substantial procedural modification being the substitution of the compounds of the present invention for the agents used in the art-recognized protocols.

The compounds of the present invention provide a method for treating fungal infections and pre-cancerous or cancerous conditions. As used herein the term unit dosage refers to a quantity of a therapeutically-effective amount of a compound of the present invention that elicits a desired therapeutic response. As used herein the phrase “therapeutically-effective amount” means an amount of a compound of the present invention that prevents the onset, alleviates the symptoms, or stops the progression of a fungal infection or pre-cancerous or cancerous condition. The term “treating” is defined as administering, to a subject, a therapeutically-effective amount of at least one compound of the present invention, both to prevent the occurrence of a fungal infection or pre-cancer or cancer condition, or to control or eliminate a bacterial or fungal infection or pre-cancer or cancer condition. The term “desired therapeutic response” refers to treating a recipient subject with a compound of the present invention such that a fungal infection or pre-cancer or cancer condition is reversed, arrested or prevented in a recipient subject.

The compounds of the present invention can be administered as a single daily dose or in multiple doses per day. The treatment regime may require administration over extended periods of time, e.g., for several days or for from two to four weeks. The amount per administered dose or the total amount administered will depend on such factors as the nature and severity of the infection, the age and general health of the recipient subject, the tolerance of the recipient subject to the compound and the type of the fungal infection, or type of cancer.

A compound according to this invention may also be administered in the diet or feed of a patient or animal. The diet for animals can be normal foodstuffs to which the compound can be added or it can be added to a premix.

The compounds of the present invention may be taken in combination, together or separately with any known clinically approved antibiotic, anti-fungal or anti-cancer to treat a recipient subject in need of such treatment.

### EXAMPLE 1

### Fermentation

A vial containing frozen spores of Streptomyces melanosporofaciens NRRL B-12234 was taken out of freezer and kept on dry ice. Under aseptic conditions, a loopfull of the frozen spores was taken and streaked on the surface of tomato paste-oat meal agar (ATCC medium 1360) plate and incubated at 28° C. for 5-7 days until sporulation occurred.

To prepare a vegetative culture, 1-2 loopfull of the spores obtained from the surface of the tomato paste-oat meal agar plate were transferred to a 125-ml flask containing 25 ml of sterile medium ITSB, containing 30 g Trypticase Soy Broth (BD), 3 g yeast extract (Difco), 2 g MgSO4, 5 g glucose, and 4 g maltose made up to one litre with distilled water. This vegetative culture was incubated at 28° C. for about 60 hours on a shaker set at 250 rpm.

Ten ml of the vegetative culture was used to inoculate 2-L baffeled flasks each containing 500 ml of sterile production medium selected from Table 1. The fermentation batches were incubated aerobically under stirring (250 rpm) at 28° C. for 4 days.

The fermentation protocol was repeated using each of the production media designated AA, AB, ET, FA, JA and VB described in Table 1 below, wherein all ingredients are expressed in g/L except for the Nal in medium ET that is expressed in mg/L, and wherein the pH is adjusted as marked prior to the addition of CaCO₃.

TABLE 1

| Component | AA | AB | ET | FA | JA | VB |
|---|---|---|---|---|---|---|
| pH | | | | 7.0 | 7.3 | 7 |
| Glucose | 10 | | | 10 | | 10 |
| Sucrose | 15 | | | | | 20 |
| Mannitol | | 25 | | | | |
| Cane molasses | | | 60 | 15 | | 20 |
| Soluble starch | | 25 | 20 | | | |
| Corn starch | | | | | 30 | |
| Corn dextrin | 40 | | | | | |
| Potato dextrin | | | | 40 | | |
| Corn steep liquid | | | | | 15 | |
| Malt extract | | | | | 35 | |
| Pharmamedia | | | | | 15 | |
| Glycerol | | 25 | | | | |
| N-Z Amine A | 10 | | | 10 | | |
| Fish meal | | | 20 | | | |
| Soytone Peptone | | | | | | 5 |
| Glutamine | | 5.84 | | | | |
| Arginine | | 1.46 | | | | |
| MgSO₄.7H₂O | 1 | 0.5 | | 1 | | |
| MgCl₂.6H₂O | | | | | | |
| CaCO₃ | 2 | | 2 | 2 | 2 | 2.5 |
| NaCl | | 1 | | | | |
| KH₂PO₄ | | 1 | | | | |
| Na₂HPO₄ | | | | 3 | | |
| Nal | | | 0.5 | | | |
| CuSO₄.5H₂O | | | 0.1 | | | |
| Trace element | | 2 | | | | |

wherein the trace element is a solution containing 0.1 g Fe SO₄.7H₂O; 0.01 MnSO₄.H₂O; 0.01 g CuSO₄.H₂O; 0.01 Zn SO₄.7H₂O; one drop concentrated H₂SO₄; dissolved in 100 ml deionized distilled H₂O.

Compound 1 was produced by the microorganism when grown on each of the media compositions M, AB, ET, FA, JA and VB. The preferred medium for production of Compound 1 was JA. Compound 2 was produced by the microorganism when grown on any one of media compositions AA, AB, ET, FA, JA and VB. The preferred medium for production of Compound 2 is AB.

### EXAMPLE 2

### Isolation

Thirty minutes prior to harvest of the compound from the fermentation broth of the baffelled flasks of Example 1, regenerated, water-washed, Diaion HP-20® in a quantity of wet-packed volume equal to 12% of the initial fermentation beer volume was added to the whole fermentation broth of Example 1 and modest agitation was continued for 30 minutes. At harvest the fermentation broth of each of Examples 1 was centrifuged and the supernatant was decanted from the resin and mycelia pellet. The pellet was resuspended in water (half the original fermentation beer volume), agitated mildly and recentrifuged, and the surpernatant was decanted from the residue. The residue was washed a second time in the same manner with water and a third time in the same manner with water:methanol (3:1 v/v) each at 50% original fermentation beer volume to obtain a re-washed residue. The re-washed residue was further washed in the same manner three more times with 20% original fermentation beer volume, followed by two more washes with methanol:water (1:1 v/v), and a single final wash with methanol:water (7:3 v/v) to obtain a well-washed residue. The well-washed mycelia: resin residue was extracted three times with 100% methanol, each extract being at 20% original beer volume. The three extracts were combined and concentrated under vacuum on a rotary evaporator, to dryness. The semi-solid residue of crude Compounds 1 and 2 represented greater than 90% of the respective compounds produced and these two compounds comprised about 25% of the total residue.

The semi-solid residues of crude Compound was purified using a Waters Xterra® preparative MS C-18 column with 10 μm packing of dimensions 19 mm diameter×150 mm length was used with a gradient from 5 mM aqueous ammonium bicarbonate to acetonitrile according to Table 2;

TABLE 2

| Time (min) | % Aqueous | % Acetonitrile |
|---|---|---|
| 0 | 95 | 5 |
| 10 | 45 | 55 |
| 15 | 0 | 100 |
| 30 | 95 | 5 |

The eluate was monitored at 390 nm. For each of Compound 1 and Compound 2, a single run was loaded with about 52 mg of crude residue in 1 ml of methanol, and a conservative cut of the peak eluting at 10.1 minutes, with a flow rate of 9 ml/min, gave 12 mg of each product of the respective compounds with about 95% purity.

### EXAMPLE 3

### Structure Data of Compound 1

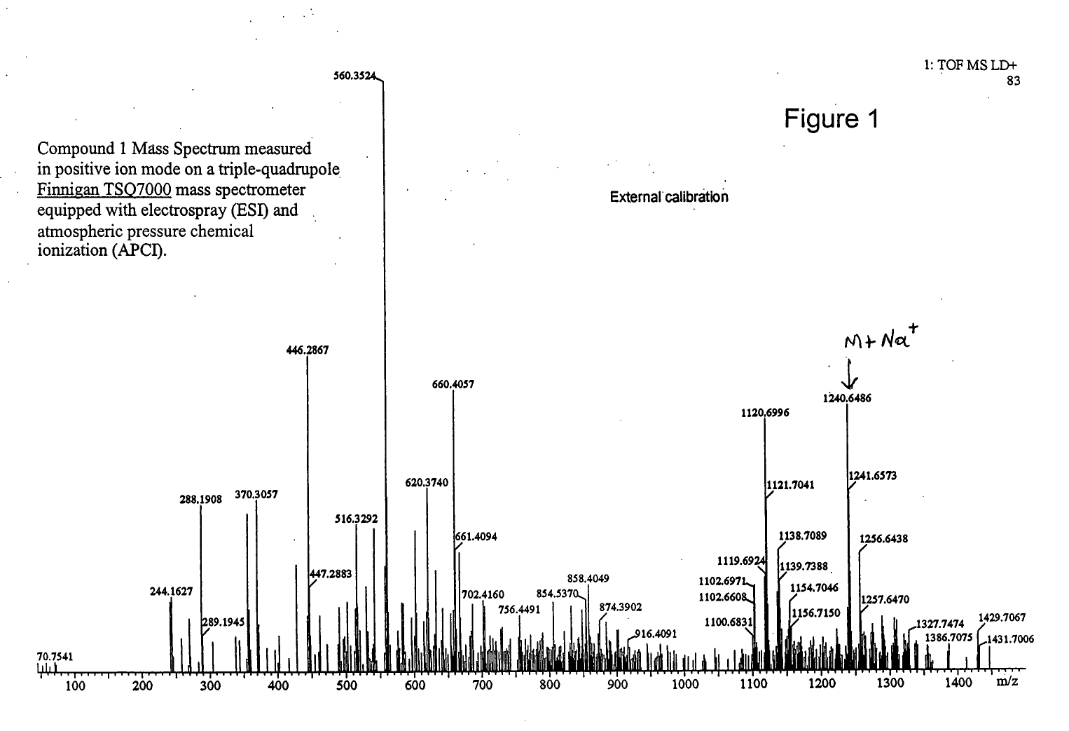

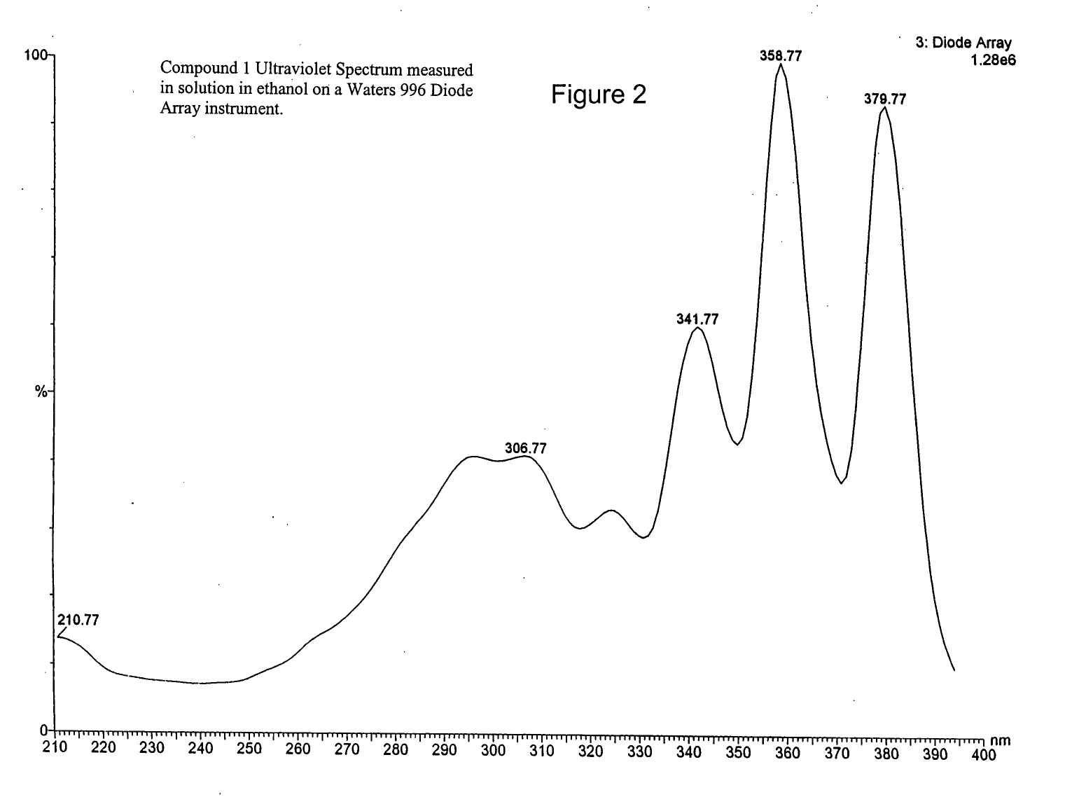

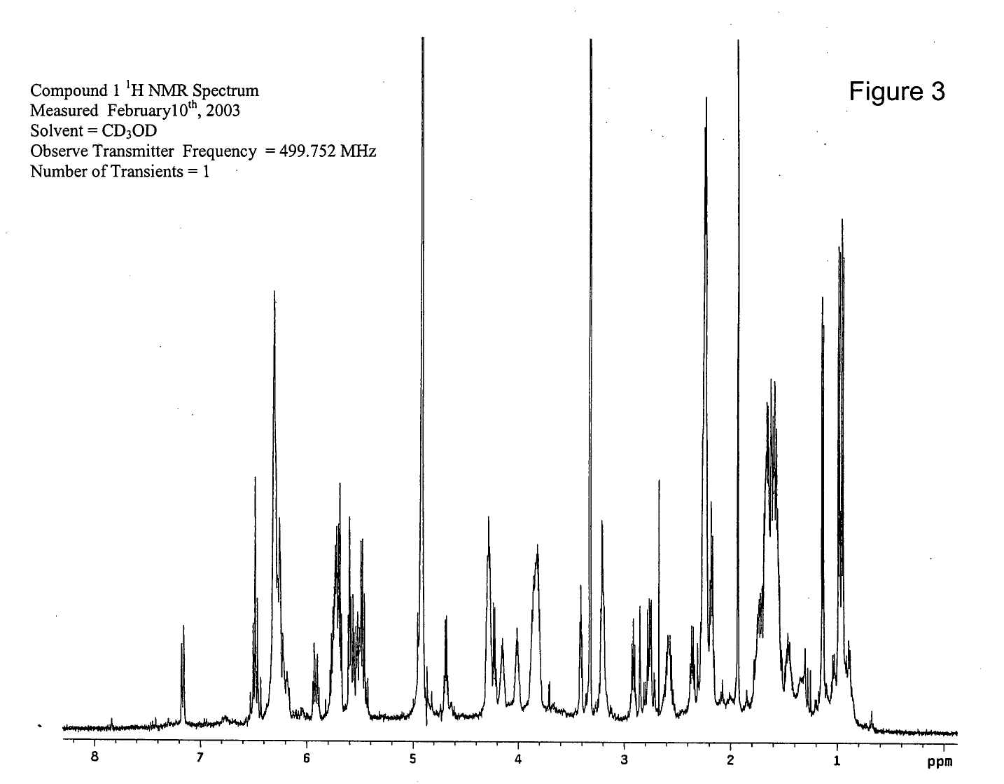

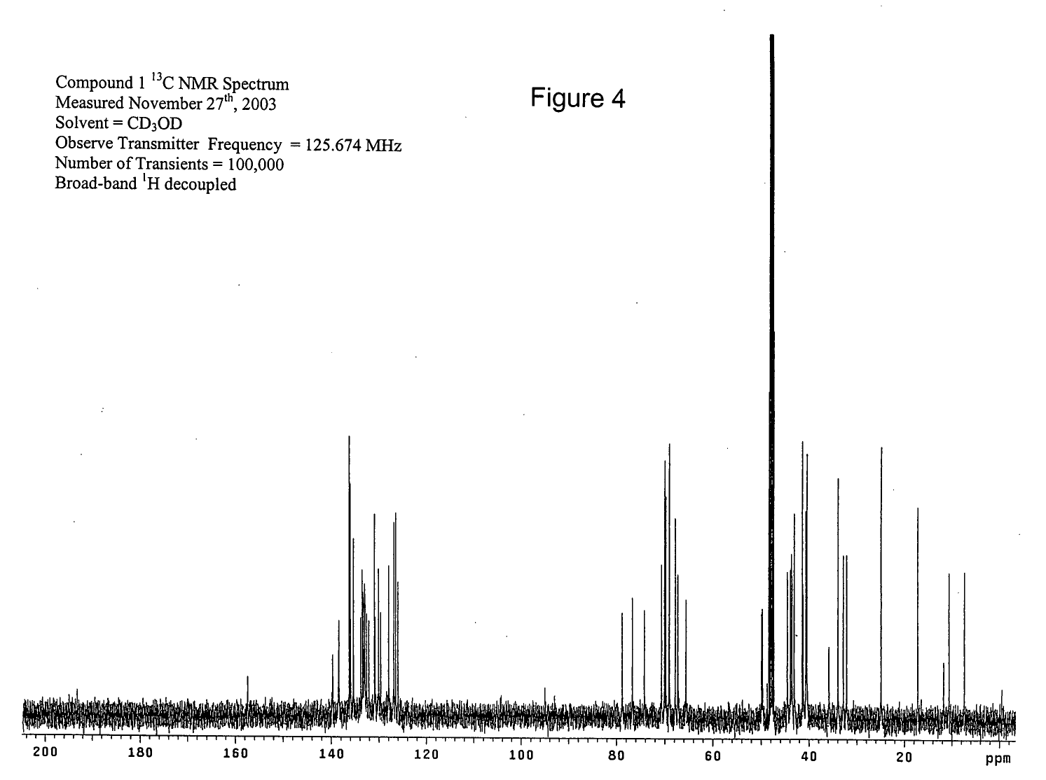

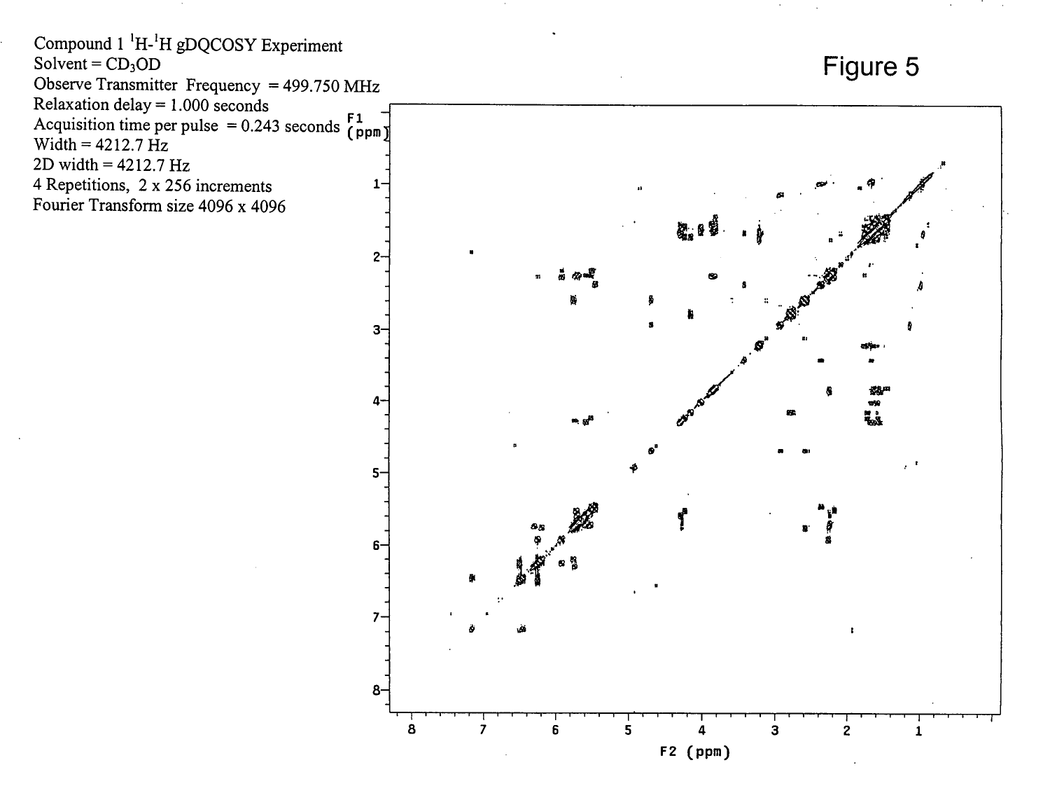

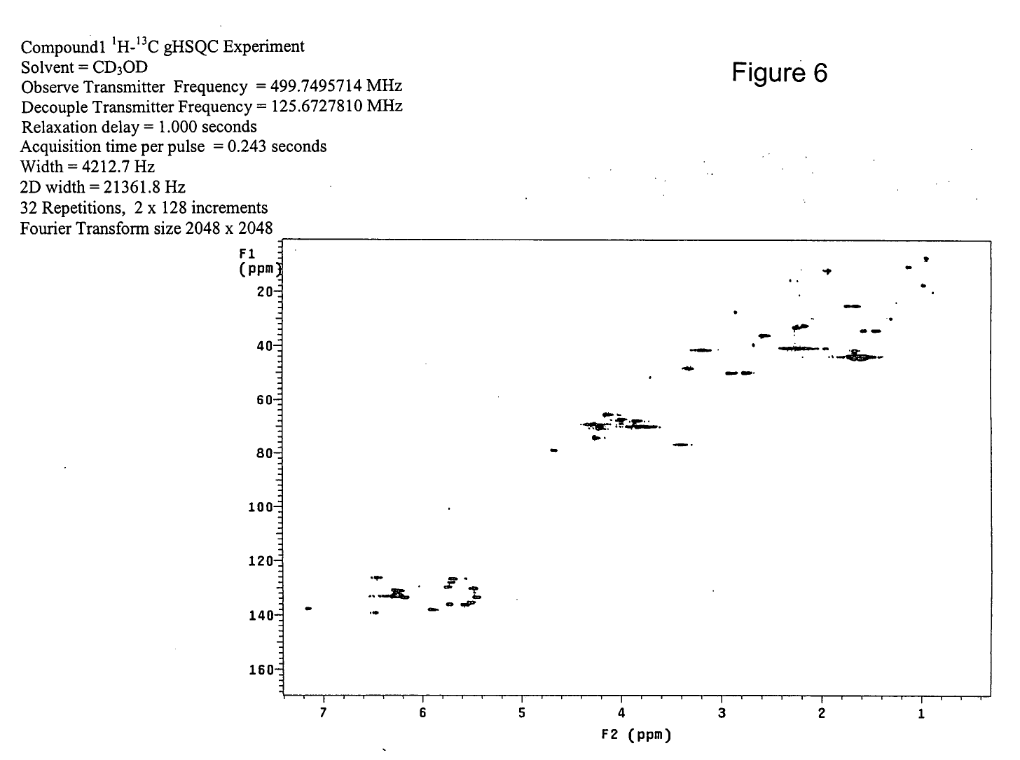

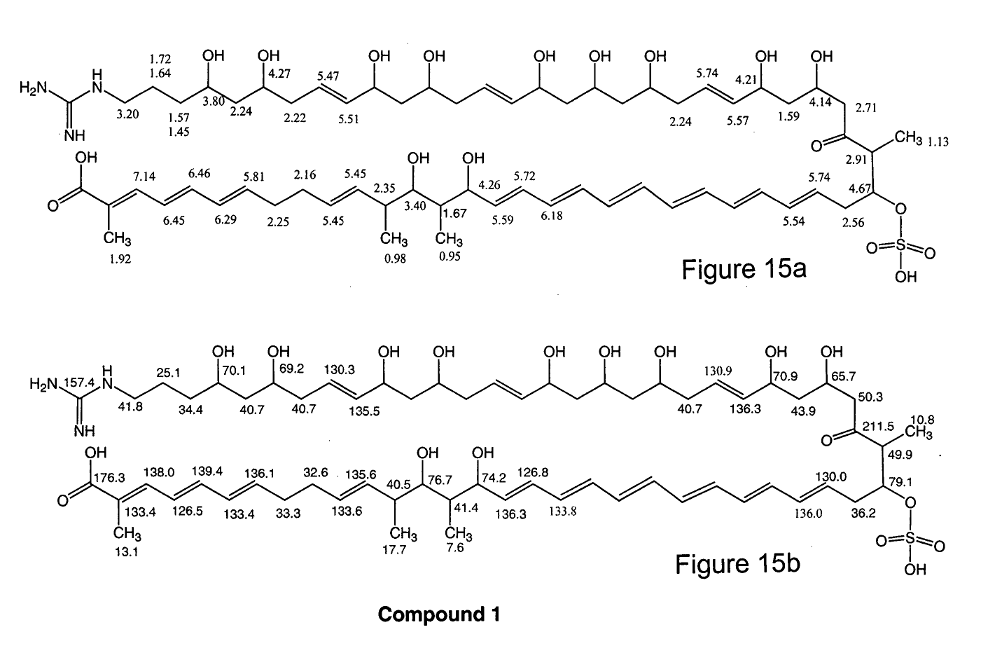

The structure of Compound 1 was determined from spectroscopic data including NMR spectroscopy. The molecular weight was determined by electrospray mass spectrometry to be 1217 as shown in the mass spectrum of FIG. 1. The mass spectrum of FIG. 1 was measured in positive ion mode on a triple-quadrupole Finnigan™ TSQ7000 mass spectrometer equipped with electrospray (ESI) and atmospheric pressure chemical ionization (APCI). The identification of the m/z 1240.6486 peak as the M+Na⁺ ion is supported by spectra measured in the negative ion mode. The UV spectrum of Compound 1, measured on a solution in ethanol using a Waters™ 996 Diode Array instrument, (FIG. 2) showed λₘₐₓ at about 295, 307, 324, 342, 359, and 380 nm. The last three intense peaks are a characteristic chromophore of linear conjugated hexaenes. The ¹H NMR spectrum for Compound 1 (FIG. 3) and the multidimensional pulse sequences experiments, gDQCOSY, gHSQC, and gHMBC (FIGS. 5, 6, and 7 respectively) were measured at about 500 MHz on a sample dissolved in MeOH-d4. The 13 C NMR spectrum (FIG. 4) was measured at 125 MHz on a sample dissolved in MeOH-d4. FIGS. 15a and 15b show selected ¹H NMR and ¹³C NMR assignments for Compound 1.

The structure of the Compound 1 was completed and confirmed by genomic analysis of the biosynthetic locus for production of the Compound 1 and Compound 2 in Streptomyces melanosporafaciens strain (NRRL B-12234). The biosynthetic locus was obtained using the genome scanning technology described in detail in U.S. Ser. No. 10/232,370.

Biosynthesis of Compound 1 and Compound 2 involves the action of a multimodular type I polyketide synthase system formed by nine type 1 polyketide synthase (PKS) genes. Type I PKSs are large modular proteins that condense acyl thioester units in a sequential manner. PKS systems consist of one or more polyfunctional polypeptides each of which is made up of modules. Each type I PKS module contains three domains: a β-ketoacyl protein synthase (KS), an acyltransferase (AT) and an acyl carrier protein (ACP). Domains conferring additional enzymatic activities such as ketoreductase (KR), dehydratase (DH) and enoylreductase (ER) can also be found in the PKS modules. These additional domains result in various degrees of reduction of the β-keto groups of the growing polyketide chain. Each module is responsible for one round of condensation and reduction of the β-ketoacyl units. As a result, there is a direct correlation between the number of modules and the length of the polyketide chain as well as between the domain composition of the modules and the degree of reduction of the polyketide product.

The order of biosynthetic modules within a PKS system as well as the number and type of catalytic domain within each module determine the order and type of structural and functional elements in the resulting polyketide molecule. Thus, there is a direct relationship between the structure of the PKS and the polyketide it produces, making it possible to determine the backbone structure of a polyketide from the PKS. Those skilled in the art will appreciate that the polyketide core structure may be determined based on the architecture of the PKS modules and domains found in a given biosynthetic pathway (Staunton and Weissman, Nat. Prod. Rep., 2001 18, 380-416 (2001); Hopwood, Chem. Rev., 97, 2465-2497 (1997)).

The domain architecture of the 28 modules present in the nine PKS genes was determined. The loading module contained only an ACP domain, (module 0), whereas each of the remaining 27 modules contained domains KS, AT and ACP in various combinations with KR, DH and ER domains. The presence of a thioesterase (TE) domain in module 27 identified it as the last module in the biosynthesis of the polyketide chain. Multiple amino acid alignments were performed to identify functional KS, AT, DH, ER, KR, ACP and TE domains, as evidenced by overall similarity of related domains and a high conservation of protein regions and of amino acid residues important for catalytic activity. The TE domain that is present only once in the PKS system was compared to prototypical domains from the nystatin type I polyketide system (Brautaset, supra).

Phylogenetic analysis of the AT domains in the PKS system was conducted to assess the nature of the β-keto acyl units that are incorporated in the polyketide chain. The AT domains of the PKS system were compared to two domains, AAF71775mod 1 and AAF71776mod 3 (National Center for Biotechnology Information (NCBI) nonredundant protein database), derived from the nystatin PKS system (Brautaset, supra) and responsible for the incorporation of methylmalonyl-CoA and malonyl-CoA respectively.

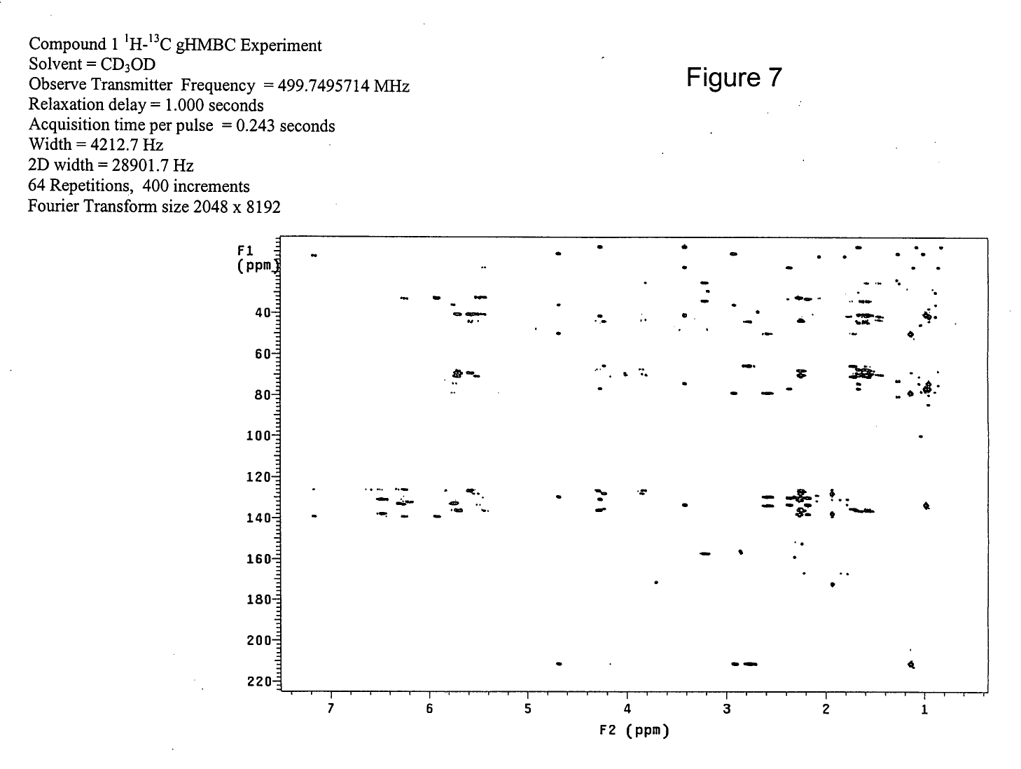

The structural difference between Compound 1 and 2, i.e. the presence of a sulfate group in Compound 1 was established by the presence of a sulfate transferase in the biosynthetic locus. The position of the sulfate group was determined by the position of the carbinol proton signal at 4.67 ppm in the ¹H NMR spectrum of Compound 1 and the carbon signal at 79.1 ppm in the ¹³C NMR spectrum, both indicating that this carbon is substituted with a modified oxygen. The gHMBC experiment of Compound 1 (FIG. 7) clearly identifies long range coupling to the proton at 4.67 ppm with each of the two β-carbons at 36.2 and 49.9 ppm and each of the three λ-carbons at 10.8, 136.0 and 211.5 ppm. The lack of any apparent coupling to an ester carbonyl strongly indicates that this oxygen must be bonded to another hetero atom.

### EXAMPLE 4

### Structure Data of Compound 2

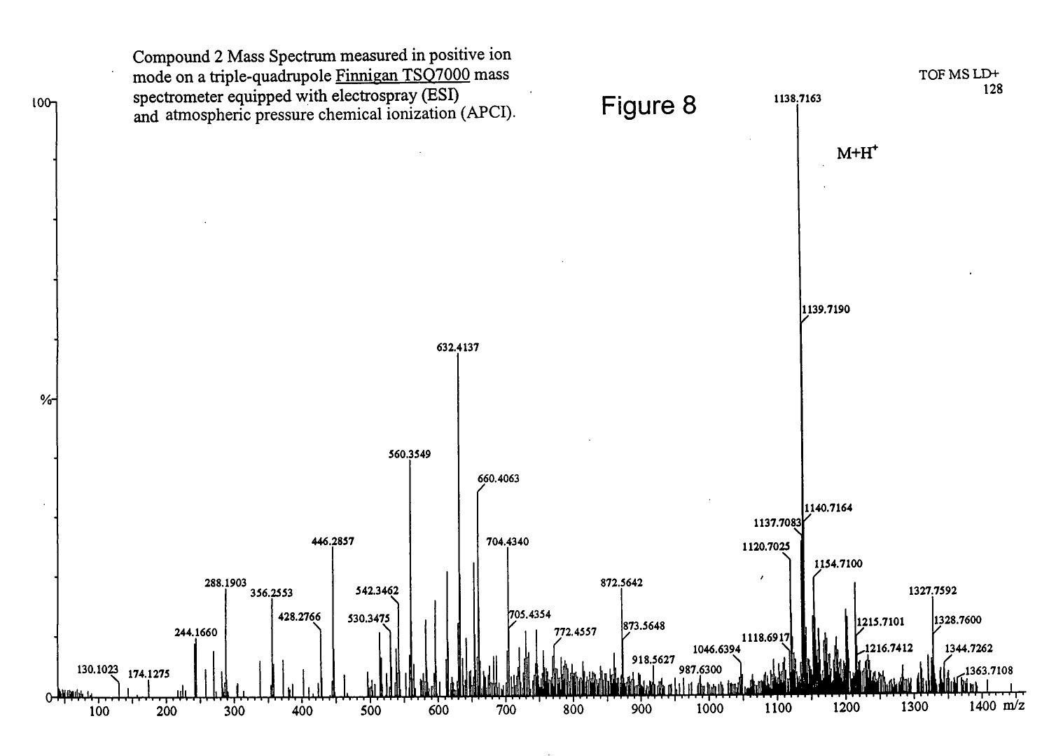

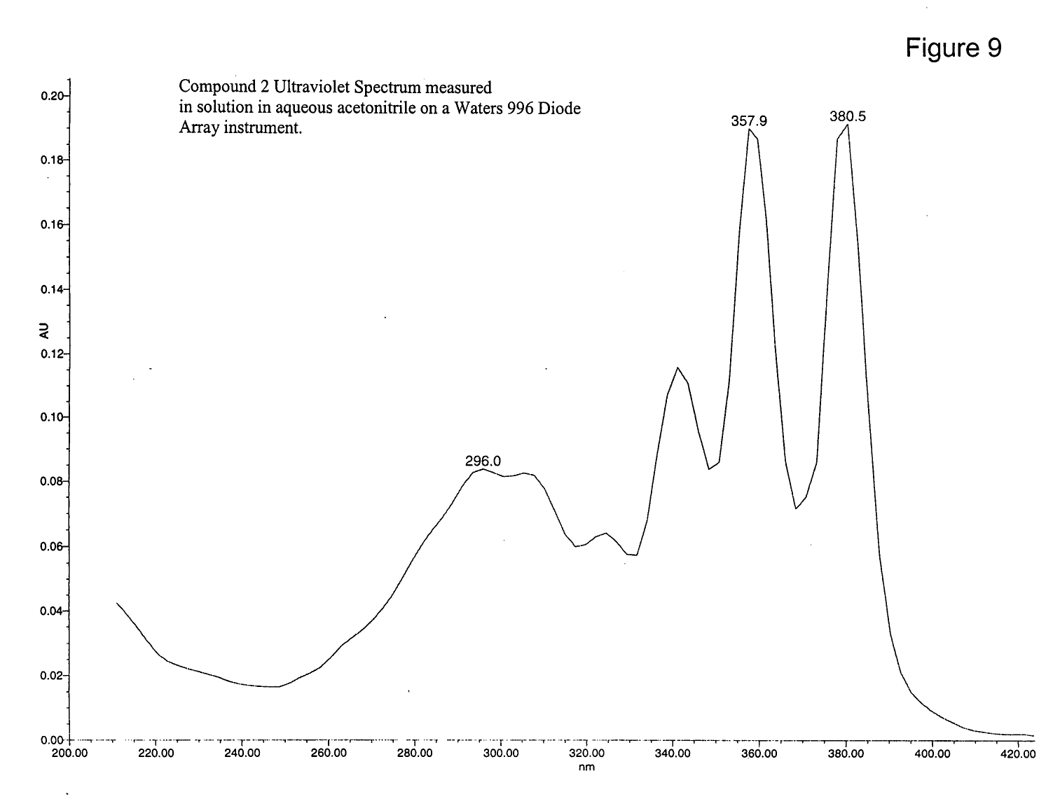

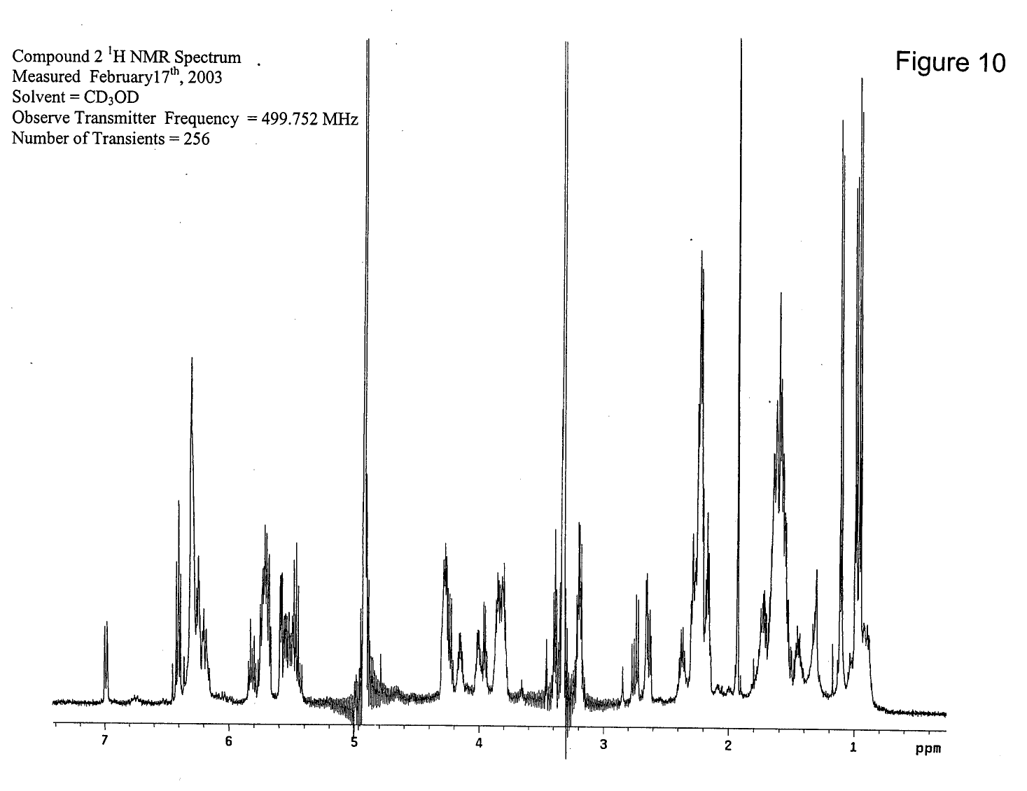

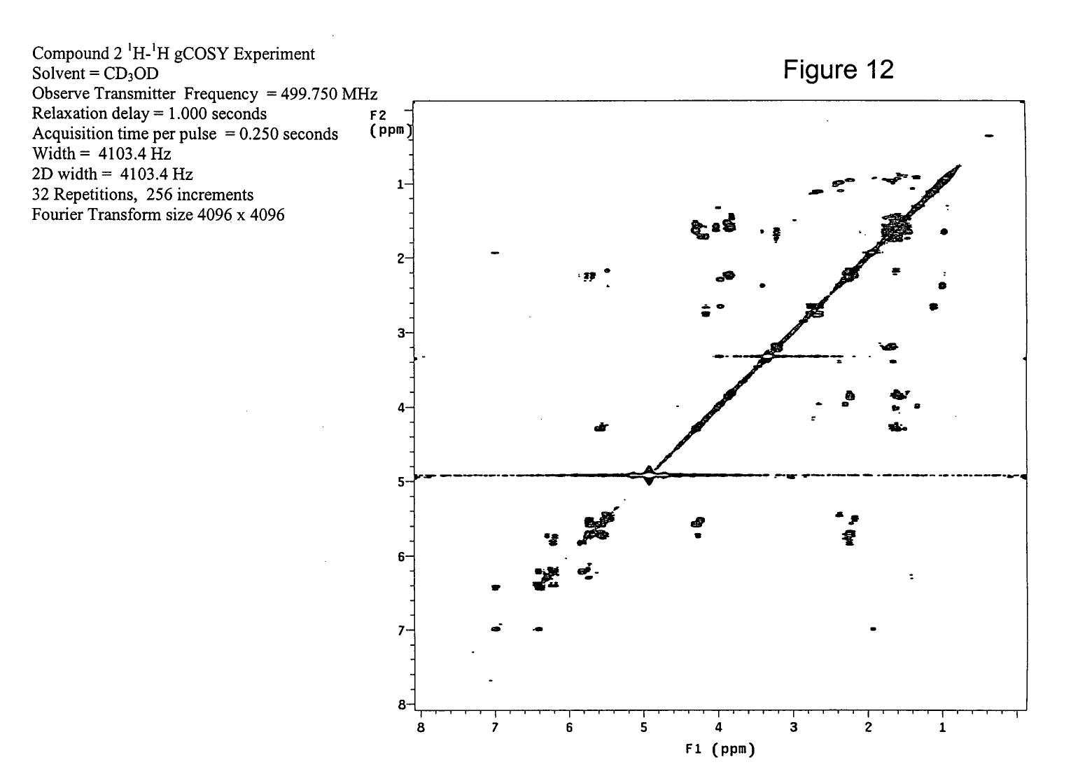

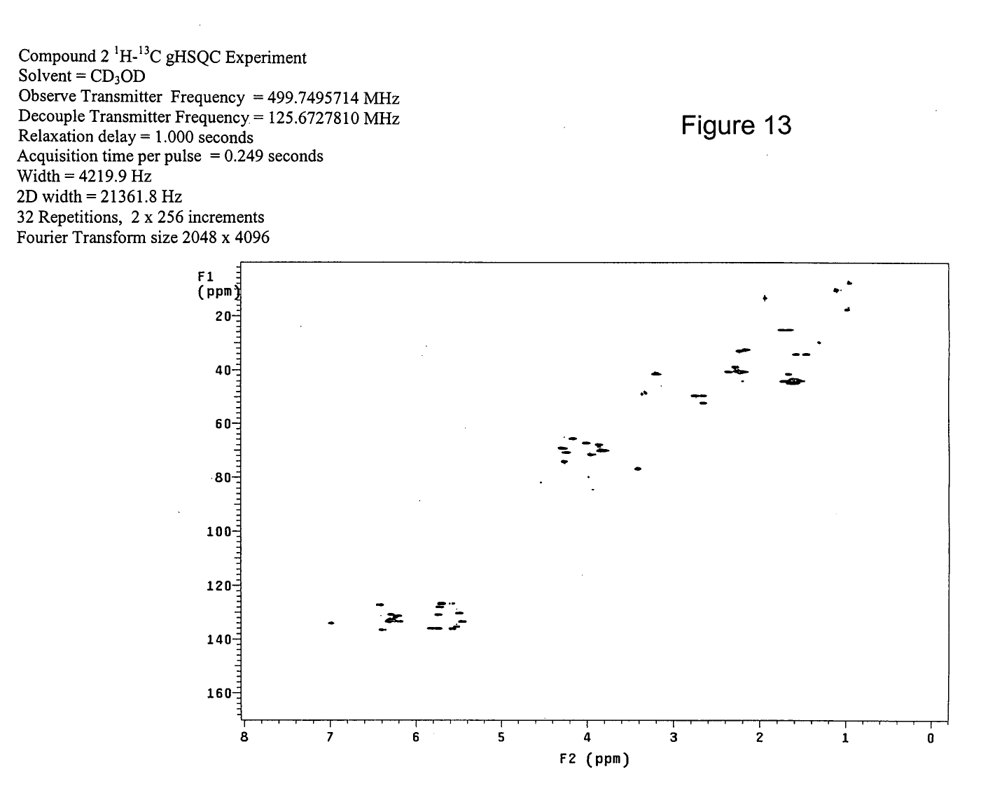

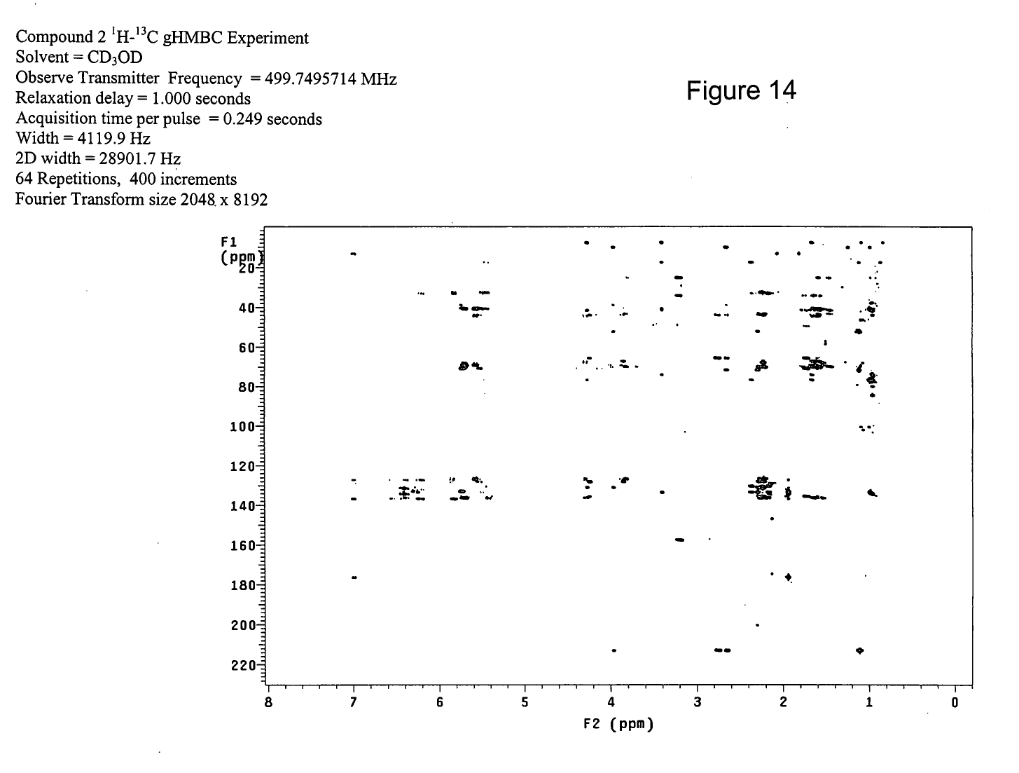

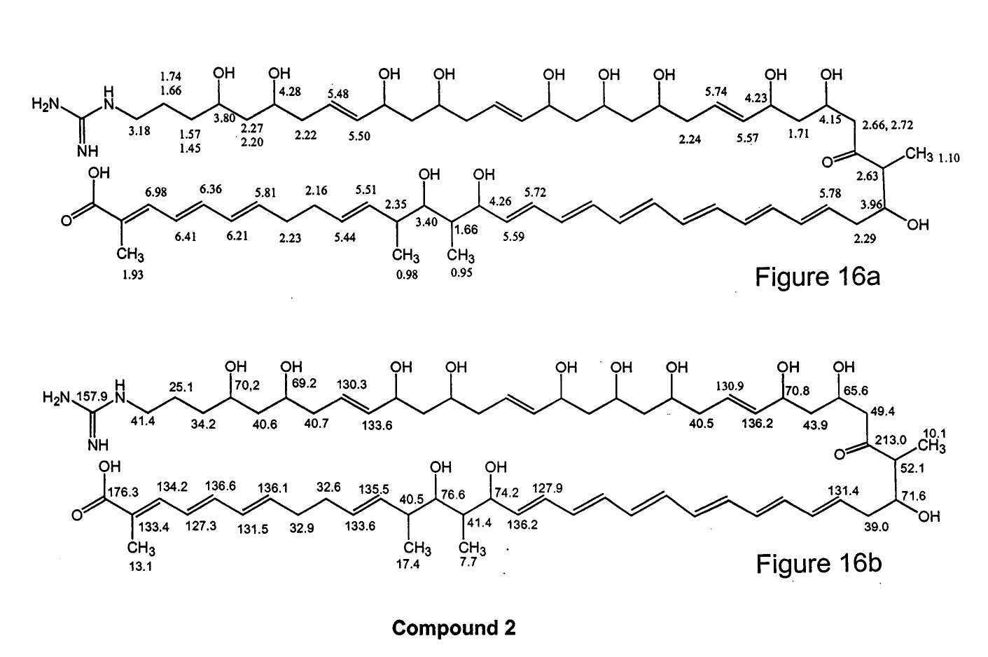

The structure of Compound of 2 was determined from spectroscopic data including NMR spectroscopy. The molecular weight was determined by electrospray mass spectrometry to be about 1137, as shown in the mass spectrum of FIG. 8. The mass spectrum of FIG. 8 was measured in positive ion mode on a triple-quadrupole Finnigan™ TSQ7000 mass spectrometer equipped with electrospray (ESI) and atmospheric pressure chemical ionization (APCI). The UV λₘₐₓ for Compound 2 were determined to be about 296, about 307, about 325, about 341 and about 358 and about 380, as shown in the ultraviolet spectrum of FIG. 9. The ultraviolet spectrum for Compound 2 was measured in solution in aqueous acetonitrile on a Waters™ 996 Diode Array instrument. The ¹H NMR spectrum for Compound 2 (FIG. 10), and the multidimensional pulse sequences experiments, gCOSY, gHSQC, and gHMBC. (FIGS. 12, 13 and 14) were measured at 500 MHz on a sample dissolved in MeOH-d4. The ¹³C NMR spectrum (FIG. 14b), was measured at 125 MHz on a sample dissolved in MeOH-d4. FIGS. 16a and 16b show selected ¹H NMR and ¹³C NMR assignments for Compound 2.

The structure of the Compound 2 was completed and confirmed by the genomic analysis of the biosynthetic locus for production of the Compound 2 in Streptomyces melanosporafaciens strain (NRRL B-12234). As described in Example 3, the biosynthetic locus contains a type I PKS system. The domains and the reactions they carry out are well documented in the literature (see, Staunton and Weissman, supra; and Hopwood, supra), and those skilled in the art will readily appreciate that it was possible to confirm the structure of Compound 2 by the architecture of the PKS system and analysis of the proteins present in the biosynthetic locus, as described above in Example 3.

### EXAMPLE 5

### Minimal Inhibitory Concentration (MIC) Determination

The MIC determination for fungal organisms was performed using the broth microdilution assay adapted from National Committee for Clinical Laboratory Standards (NCCLS) M27-A (Vol. 17 No. 9, 1997), Reference Method for Broth Dilution Antifungal Susceptibility Testing of Yeasts; Approved Standard guidelines: M27-A.

Microdilution of Test Compound: An initial stock solution of the test compound was prepared in DMSO at 100×. The highest concentration used in the assay was 3.2 mg/ml. This solution was used to prepare a two-fold dilution series over 11 points of 100× solutions in DMSO. These solutions were diluted 50× in test medium (RPMI-1640 medium) to give a set of eleven (11) 2× media/compound solutions (“dilution” 12 was the media/solvent alone, control). One hundred microlitres (100 ul) of each of the eleven 2× solutions was aliquoted into the corresponding well of a 12-well row, with the final well reserved for medium/solvent alone control.

Inoculum Preparation: To 5 ml of sterile saline in a polypropylene screw cap tube, a sufficient amount of an overnight culture was added to obtain aturbidity visually equivalent to that of a 0.5 McFarland standard. This yielded a yeast suspension of 1×10⁶ to 5×10⁶ cells per ml. The yeast suspension was vortexed for 15 seconds, diluted 1:50 in test medium, and further diluted 1:20 with test medium to obtain a 2× test inoculum, (1×10³ to 5×10³ CFU/ml). This 2× inoculum was diluted 1:1 in the final test plate and a final inoculum of 0.5×10³ to 2.5×10³ CFU/ml, was obtained.

MIC Determination: Aliquots of 100 μl of the 2× adjusted inoculum were dispensed into each set of 12 wells. The plates were incubated at 35° C. for 48 hrs. MIC readout for each indicator strain was the lowest concentration of the compound that results in absence of growth. The results of the fungal MIC assay are summarized in Table 3.

TABLE 3
Minimal Inhibitory Concentrations

| | Compound 1 | Compound 2 | Amphotericin B | Amphotericin B |
|---|---|---|---|---|
| | 24 hrs | 48 hrs | 24 hrs | 48 hrs |
| C. albicans (Calb) | 4 μg/ml | 4 μg/ml | 0.5-1 μg/ml | 1 μg/ml |
| Candida sp. (Cflu) | 8 μg/ml | 8 μg/ml | 0.5-1 μg/ml | 1 μg/ml |
| C. glabrata (Cgla) | 4 μg/ml | 4 μg/ml | 0.5-1 μg/ml | 1 μg/ml |
| C. albicans (Ctro) | 4 μg/ml | 4 μg/ml | 1 μg/ml | 2 μg/ml |
| S. cerevisiae (Scer) | 4 μg/ml | 4 μg/ml | 0.5 μg/ml | 1 μg/ml |

### EXAMPLE 6

### In Vitro Cytotoxicity Efficacy on Cancer Cell Lines

The cell lines listed in Table 4 were used to characterize the cytotoxic efficacy of Compounds 1 and 2. These cell lines were shown to be free of mycoplasma infection and were maintained on the appropriate media shown in Table 4 and supplemented with 10% heat-inactivated fetal bovine serum and 1% penicillin-streptomycin, under 5% CO₂ at 37° C. Cells were passaged two to three times per week. Cellular viability was examined by staining with trypan blue and only flasks where cell viability was >95% were used to determine cytotoxic efficacy of compounds 1 and 2.

TABLE 4

| Cell type | Origin | Medium |
|---|---|---|
| PC3 | Human prostate | Ham's F12K, |
| | adenocarcinoma | L-glutamine (1%) |
| | AR negative | |
| HCT-116 | Human colon adenocarcinoma | McCoy's 5a |
| MDA-231 | Human breast | RPMI-1640 |
| | adenocarcinoma | |
| SK-MEL-1 | Human Melanoma | Eagle's MEM, non- |
| (HTB67) | | essential amino Acid (1%), |
| | | sodium pyruvate (1%) |
| A498 (HTB- | Human Kidney carcinoma | MEM, Non-essential |
| 44) | | amino Acid (1%), |
| | | sodium pyruvate |

In Vitro Cytotoxic Efficacy Determination

Exponentially growing cells (1-3×10³ cells per 100 μl) were seeded in 96-well plates and incubated for 16 h. Cells were then exposed continuously to various concentrations of compound 1 in serum-supplemented medium. Cell survival was evaluated 96 h later by replacing the culture media with 150 μl fresh medium containing 10 mM 4-(2-hydroxyethyl)-1-piperazine ethanesulfonic acid buffer, pH 7.4. Next, 50 μl of 2.5 mg/ml of 3-(4,5-dimethylthiazo-2-yl)-2,5-diphenyltetrazolium bromide (MTT; Sigma, St. Louis, Mo.) in phosphate buffer solution, pH 7.4, was added. After 3-4 h of incubation at 37° C., the medium and MTT was removed, and 200 μl of dimethylsulfoxide was added to dissolve the precipitate of reduced MTT followed by addition of 25 μl glycine buffer (0.1 M glycine plus 0.1 M NaCl, pH 10.5). The absorbance was determined at 570 nm with a microplate reader (BIORAD). Cell survival was estimated as % of cells treated with the vehicle alone. The cytotoxic efficacy results of Compound 1 and Compound 2 are shown in Tables 5 and 6, respectively.

TABLE 5
Cytotoxic efficacy of Compound 1

| Panel: Cancer type | Cell type | IC₅₀ (μM) |
|---|---|---|
| Non-small cell lung | A549/ATCC | 12 |
| | NCI-H460 | 13 |
| Colon | HT29 | 23 |
| CNS | SNB-19 | 10 |
| Melanoma | LB1319-MEL | 11 |
| Ovarian | OVCAR-3 | 12 |
| | SK-OV3 | 15 |
| Renal | A498 | 13 |
| Prostate | PC-3 | 10 |
| | DU-145 | 8 |
| Breast | MCF7 | 18 |
| | MDA-MB-231/ATCC | 12 |
| Pancreatic | PANC-1 | 15 |

TABLE 6
Cytotoxic efficacy of Compound 2

| Panel: Cancer type | Cell type | IC₅₀ (μM) |
|---|---|---|
| Leukemia | CCRF-CEM | >25 |
| | HL-60 (TB) | 17 |
| | K562 | 8 |
| | MOLT-4 | >25 |
| | RPMI-8226 | 6 |
| | SR | 1 |
| Non-small cell lung | A549/ATCC | 7 |
| | EKVX | 8 |
| | HOP-92 | >25 |
| | NCI-H460 | 2 |
| | NCI-H522 | 2 |
| Colon | COLO 205 | 7 |
| | HCC-2998 | 0.1 |
| | HCT-116 | 2 |
| | HCT-15 | 8 |
| | HT29 | 4 |
| | KM12 | 0.8 |
| | SW-620 | 1 |
| CNS | SF-268 | 4 |
| | SF-295 | 4 |
| | SF-539 | 3 |
| | SNB-19 | >25 |
| | SNB-75 | 10 |
| | U-251 | 6 |
| Melanoma | LOX IMVI | 6 |
| | M14 | 0.5 |
| | SK-MEL-2 | 3 |
| | SK-MEL-28 | 13 |
| | SK-MEL-5 | 2 |
| | UACC-257 | 5 |
| | UACC | 8 |
| Ovarian | IGROV1 | 0.6 |
| | OVCAR-4 | >25 |
| | OVCAR-5 | 6 |
| | OVCAR-8 | 5 |
| | SK-OV3 | >25 |
| Renal | 786-0 | 8 |
| | A498 | 6 |
| | CAKI-1 | 2 |
| | RXF 393 | 0.4 |
| | SN12C | 7 |
| | TK-10 | 7 |
| | UO-31 | 8 |
| Prostate | PC-3 | 4 |
| Breast | MCF7 | 0.3 |
| | NCI/ADR-RES | 24 |
| | MDA-MB-231/ATCC | 9 |
| | MDA-MB-435 | 0.9 |
| | BT-549 | 0.9 |
| | T-47D | >25 |
| | HS 578T | |

### EXAMPLE 7

### Synthesis of Compounds 33 and 34

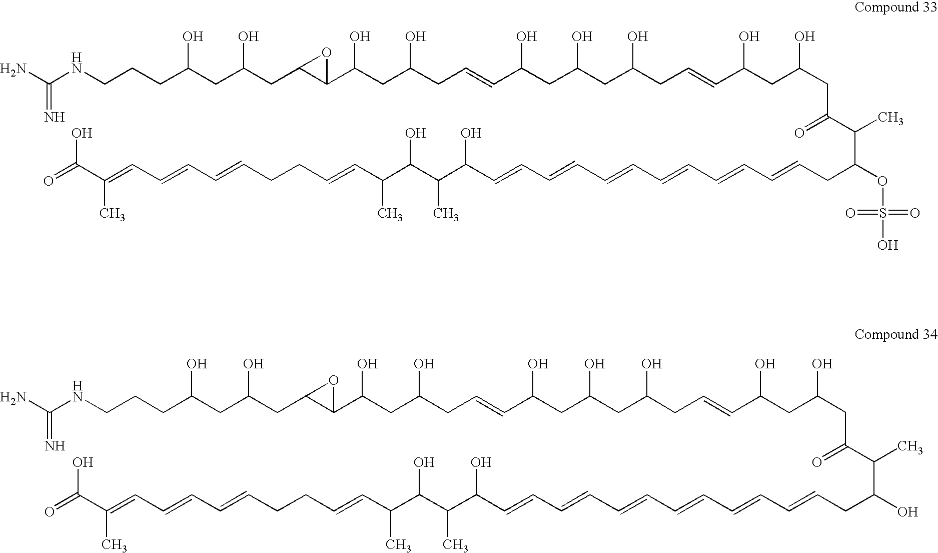

To a solution of Compound 1 or Compound 2 dissolved in tetrahydrofuran (THF) is added 1.1 equivalents of meta-chloroperbenzoic acid. The reaction is cooled in an ice bath and stirred at 0° C. for 1-2 hours. The reaction mixture is then evaporated to dryness, re-dissolved in methanol and subjected to liquid chromatography on a column of Sephadex LH-20 to isolate the Compound 33 or Compound 34 respectively.

### EXAMPLE 8

### Synthesis of Compounds 35 and 36 by Opening of the Epoxides of Compounds 33 and 34.

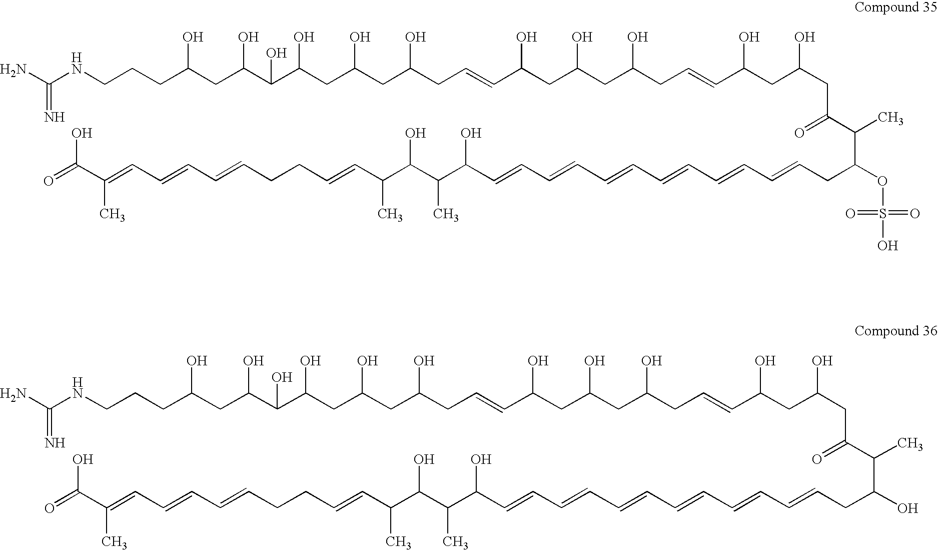

The epoxide group of Compound 33 or 34 is hydrolyzed by treatment of Compounds 33 and 34 with small quantity of aqueous hydrochloric acid (1.0 N), thereby forming the corresponding diol of the formulae 35 or 36 respectively.

### EXAMPLE 9

### Synthesis of Compounds 37 and 38 by Reduction of 29-oxo Group

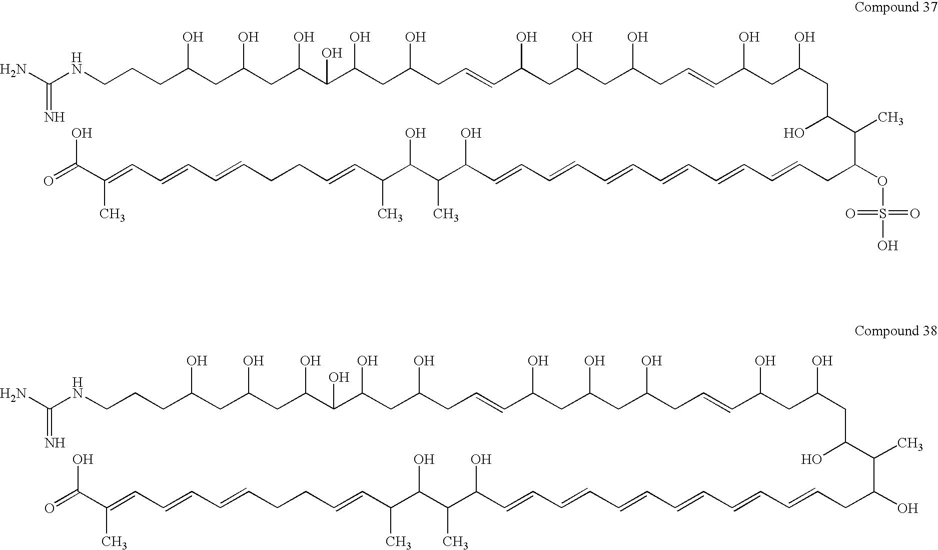

A solution of Compound 35 or 36 in acetonitrile is treated with 1.5 equivalents of NaCNBH₃. The reaction is stirred at room temperature for 1 hour. The reaction mixture is then concentrated to dryness and then taken up into methanol. The mixture is filtered and the filtrate is subjected to liquid chromatography on a column of Sephadex LH-20 to isolate the Compounds 37 or 38 respectively. Alternatively, the reduction of the oxo group at the 29-position may be effected using lithium borohydride (LiBH₄).

### EXAMPLE 10

### Synthesis of Compounds 39 and 40 by Addition of Acetal Ring at the 29-Position

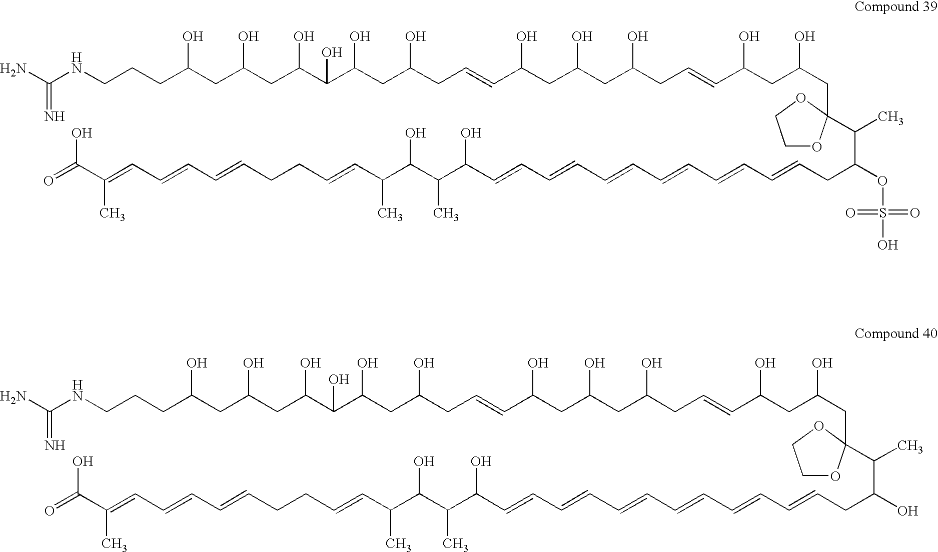

A solution of Compound 35 or 36 in tetrahydrofuran is treated with 3 equivalents of 2,2-dimethyl-1,3-dioxacyclopentane in the presence of a trace amount of toluene sulfonic acid. The reaction is stirred overnight at room temperature, evaporated to dryness and taken up into dry THF, followed by purification by liquid chromatography on a column of Sephadex LH-20. The 2,2-dimethyl-1,3-dioxacyclopentane may be synthesized by reaction of acetone with ethylene glycol in the presence of a trace of toluene sulfonic acid, over molecular sieves to remove water.

Alternatively, the addition of an acetal ring at the 29-position may be accomplished by reaction of Compound 35 or 36 with an excess of ethylene glycol in the presence of a trace of toluene sulfonic acid. The reaction is conducted over molecular sieves to remove water.

### EXAMPLE 11

### Synthesis of Compounds 41 and 42

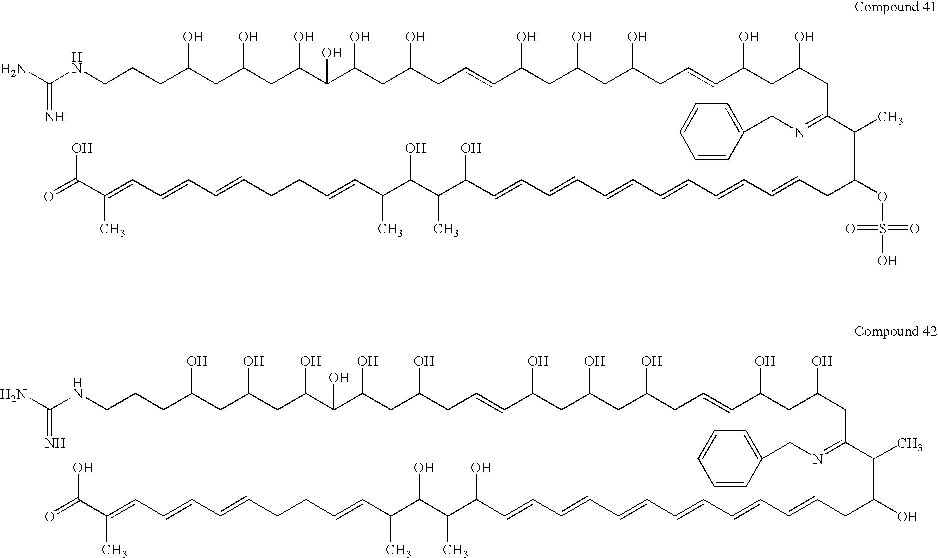

To a solution of Compounds 35 or 36 in benzene or toluene is added 10 equivalents of benzylamine. The reaction is stirred at room temperature overnight. The reaction is conducted over molecular sieves to remove water; alternatively, the water may be removed under reflux as an azeotrope with benzene or toluene using a Dean-Stark trap. The reaction mixture is concentrated under vacuum and residual reagent is removed by high vacuum at room temperature overnight.

### EXAMPLE 12

### Synthesis of Compounds 43 and 44

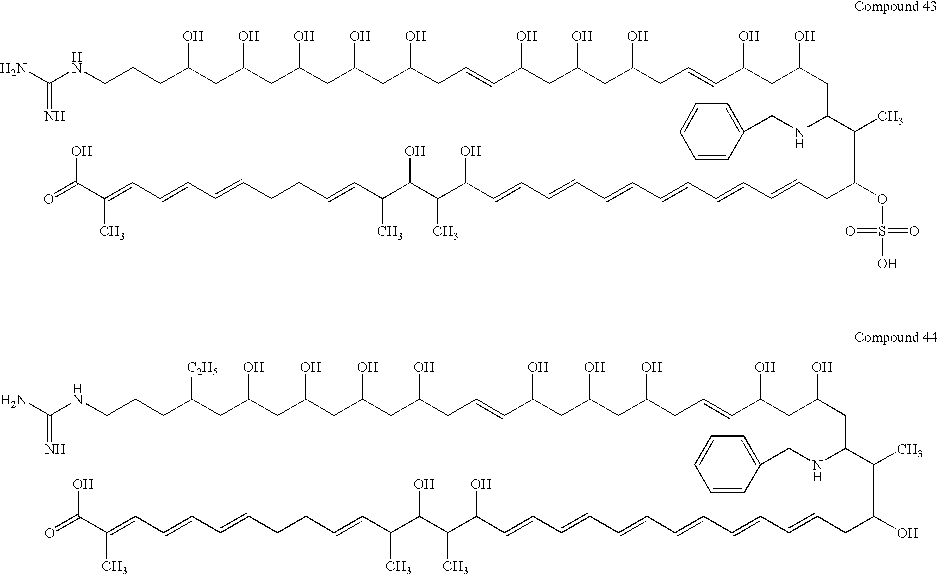

The carbon-nitrogen double bond of Compounds 41 or 42 is reduced to the amine by reaction of the compound with NaCNBH₃ or LiBH₄ (1.5 equivalents) in acetonitrile, to form compound 43 or 44 respectively.

### EXAMPLE 13

### Synthesis of Compounds 45 and 46

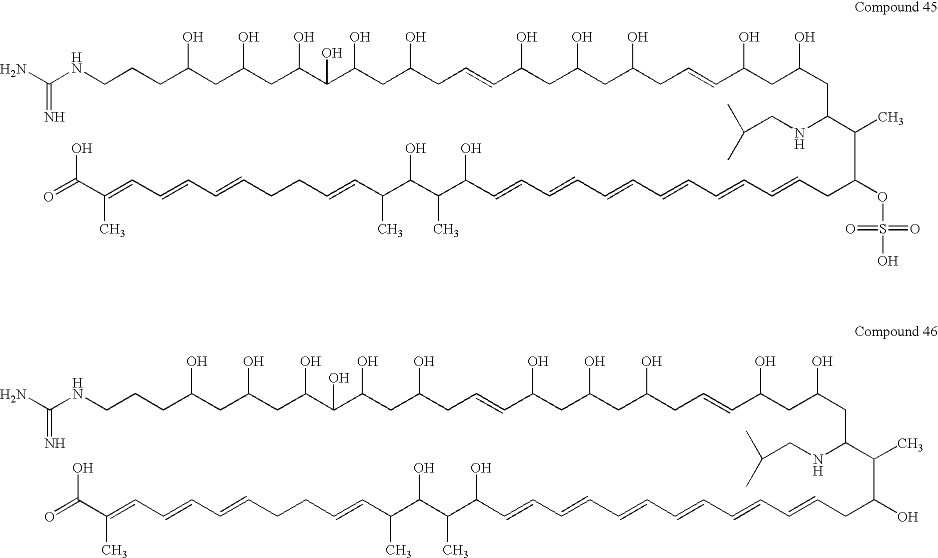

To a solution of one equivalent of Compound 35 or 36 in acetonitrile is added ten equivalents of isobutylamine. The reaction is stirred at room temperature for two hours. Benzene (1/10 volume) is added and the mixture is concentrated to dryness under vacuum on a rotary evaporator. The Schiff base is then treated with NaCNBH₃ or LiBH₄ (1.5 equivalents) in acetonitrile, to reduce the carbon-nitrogen double bond of the imine to the amine, to form Compound 45 or Compound 46 respectively.

### EXAMPLE 14

### Synthesis of Compound 47

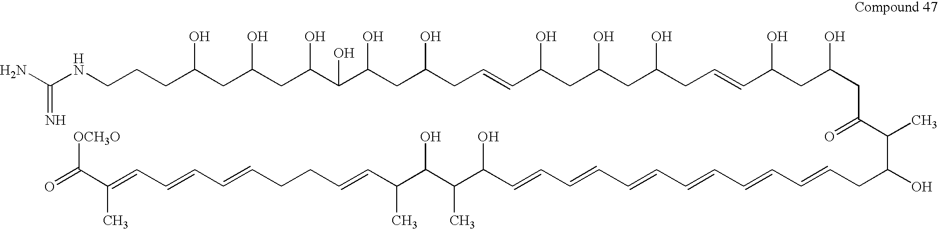

To a solution of 0.5 equivalents of Compound 36 in methanol is added 0.5 equivalents of diazomethane in diethyl ether. The reaction mixture is allowed to stand at room temperature overnight, and then the solvent is removed under vacuum to give Compound 47.

1. A compound of Formula I, or a pharmaceutically acceptable salt thereof, 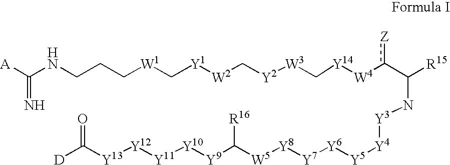
wherein,
A is selected from NHC(O)R¹, N═CR² R³, or NHR³, wherein R¹, R² and R³ are independently selected from the group consisting of H, C₁₋₆ alkyl, C₂₋₆ alkenyl, C₃₋₆ cycloalkyl, C₃₋₆ heterocycloalkyl, aryl and heteroaryl, wherein said alkyl, alkenyl, cycloalkyl, heterocycloalkyl, aryl and heteroaryl are optionally substituted with a group selected from halogen, oxo, OH, C₂₋₆ alkenyl, NO₂, NH₂, cycloalkyl, heteroaryl or aryl, said C₂₋₆ alkenyl, heteroaryl and aryl being optionally further substituted with one or more groups independently selected from halogen, OH, C₁₋₃ alkyl NO₂ or NH₂;
B is selected from 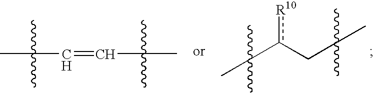
wherein R¹⁰ is OH, —OS(O)₂OH or, when the dotted line is a bond, then R¹⁰ is oxo;
D is selected from OH or C₁₋₆alkoxy optionally substituted with 1 to 2 phenyl groups, wherein the phenyl group is optionally substituted with C₁₋₆ alkyl or halo; 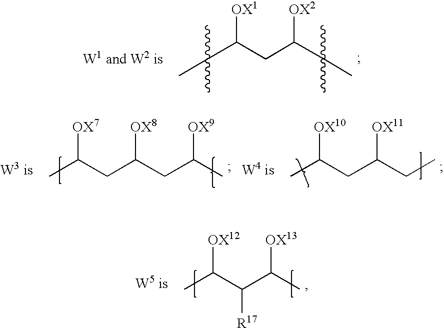
W⁵ is, wherein X¹, X², X⁷, X⁸, X⁹, X¹⁰, X¹¹, X¹² and X¹³ is each independently selected from H, —C(O)—R⁷ and a bond such that when any of two neighboring X¹, X², X⁷, X⁸, X⁹, X¹⁰, X¹¹, X¹² and X¹³ is a bond then the two neighboring oxygen atoms and their attached carbon atoms together form a six-membered acetal ring of the formula: 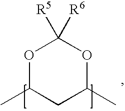
and R⁵, R⁶ and R⁷ is each independently selected from H, C₁₋₆ alkyl, C₂₋₇ alkenyl;
Y¹, Y², Y³, Y⁴, Y⁵, Y⁶, Y⁷, Y⁸, Y⁹, Y¹⁰, Y¹¹, Y¹², Y¹³ and Y¹⁴ is each independently selected from —CH₂—CH₂—, —CH═CH—, 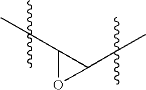
or —CH(OH)—CH(OH)—, wherein any carbon from this selection is optionally substituted with a methyl group;
Z is selected from OH, C₃₋₆ cycloalkyl, C₃₋₆ heterocycloalkyl, NHR⁸, 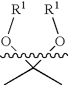
and, when the dotted line is a bond then Z is oxo, or NC₁₋₆ alkyl;
R⁸ is selected from H, C₁₋₆ alkyl, C₂₋₆alkenyl or C₃₋₆cycloalkyl; and
R¹⁵, R¹⁶ and R¹⁷ is each independently selected from H or CH₃.

2. A compound of claim 1, wherein Z is oxo.

3. A compound of claim 2 wherein A is 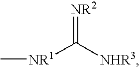
and R¹, R² and R³ are as defined in claim 1.

4. A compound of claim 3 wherein D is —OH.

5. A compound of Formula II, or a pharmaceutically acceptable salt thereof, 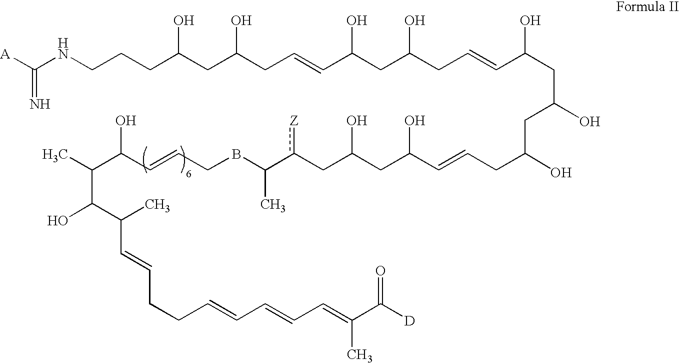
wherein A, B, D and Z are defined in claim 1.

6. A compound of claim 5 wherein Z is oxo.

7. A compound of claim 6 wherein A is 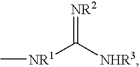
and R¹, R² and R³ are as defined in claim 1.

8. A compound of claim 7 wherein D is —OH.

9. A compound selected from the group consisting of:

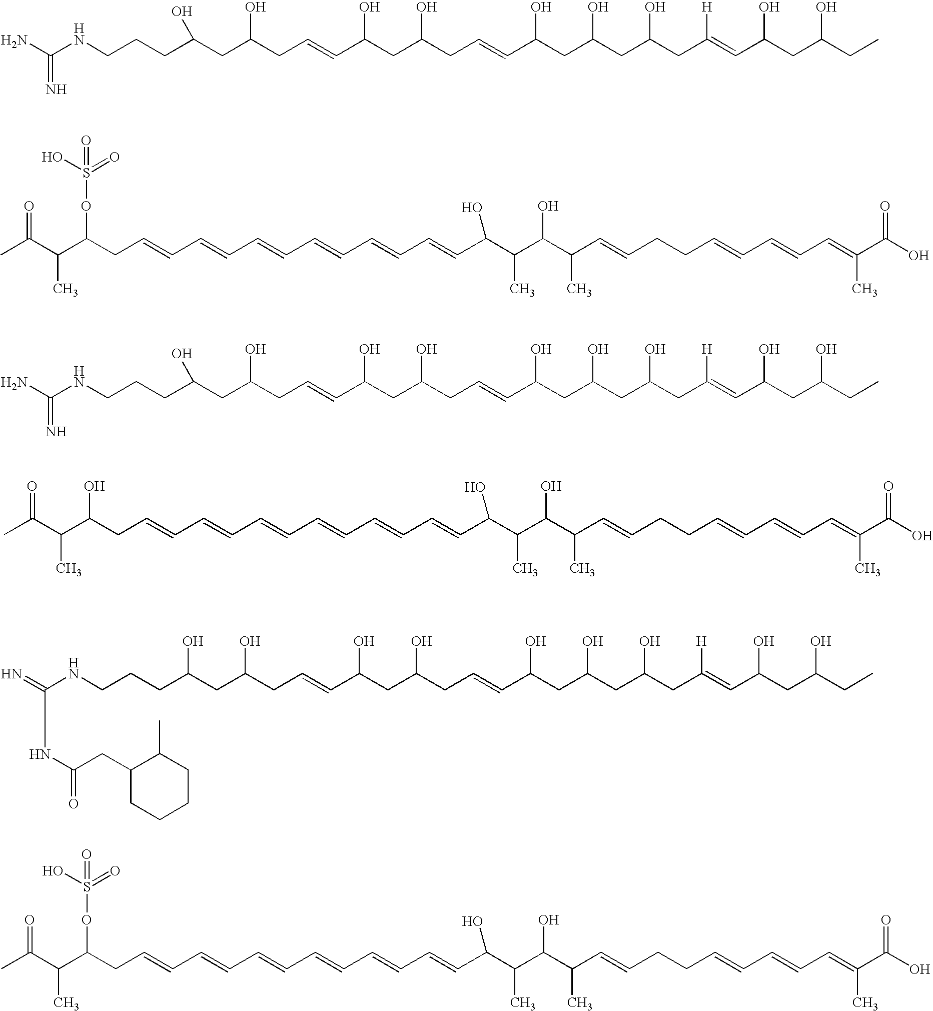

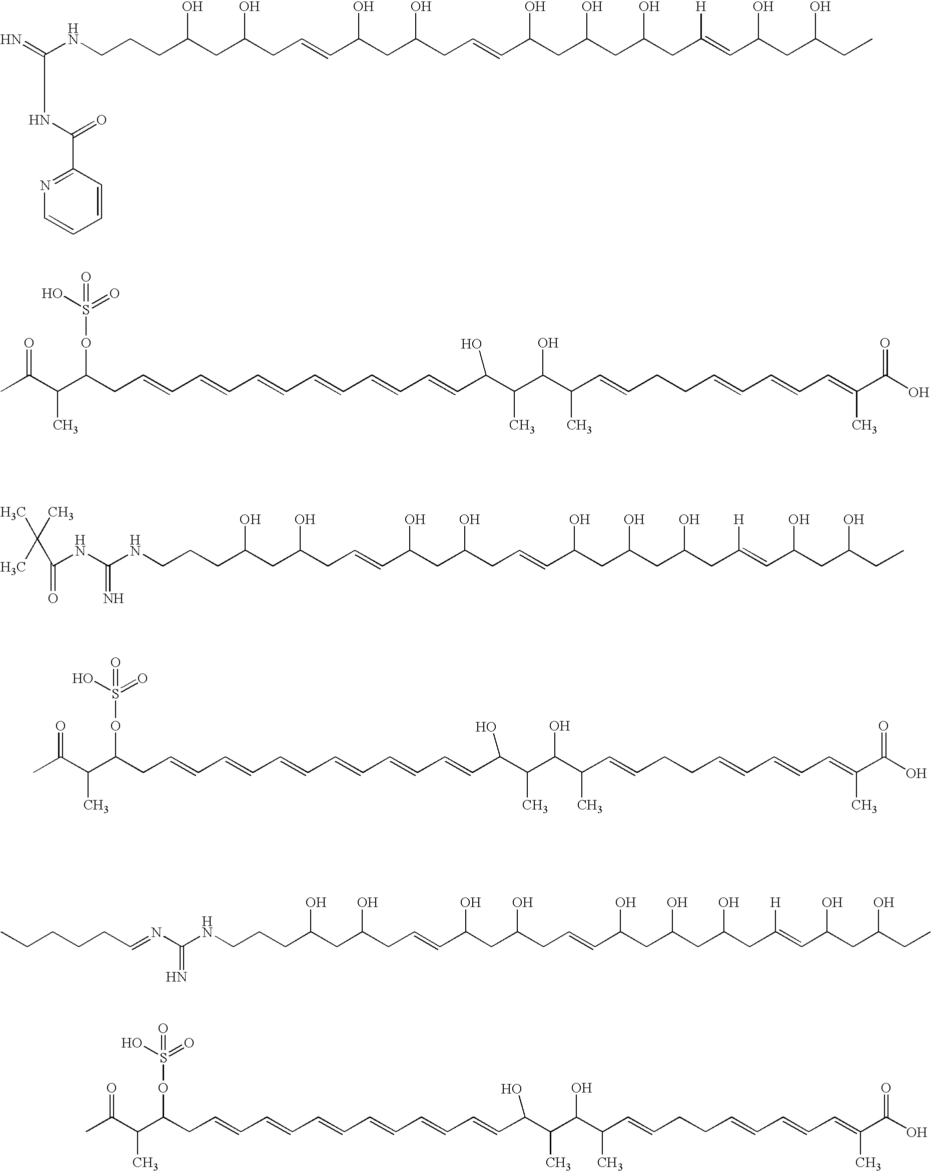

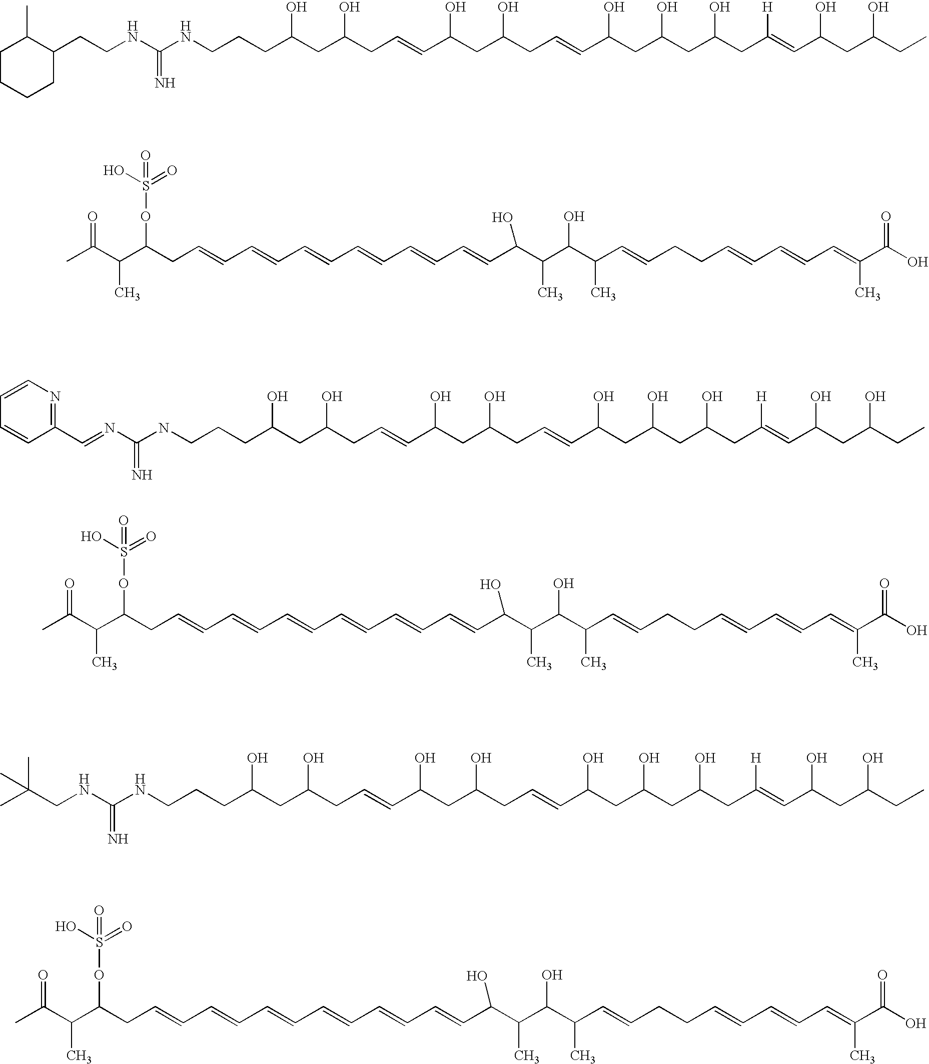

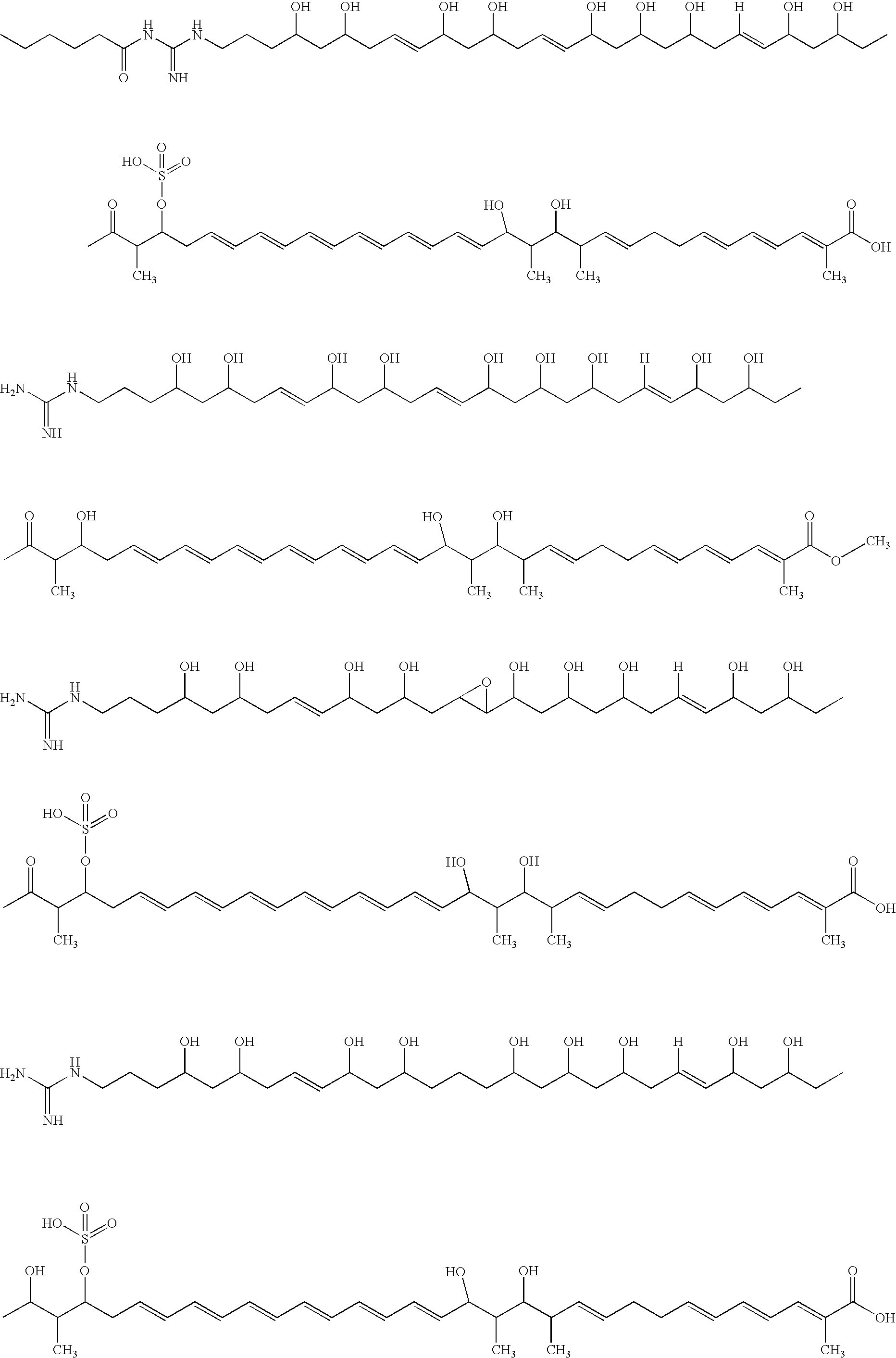

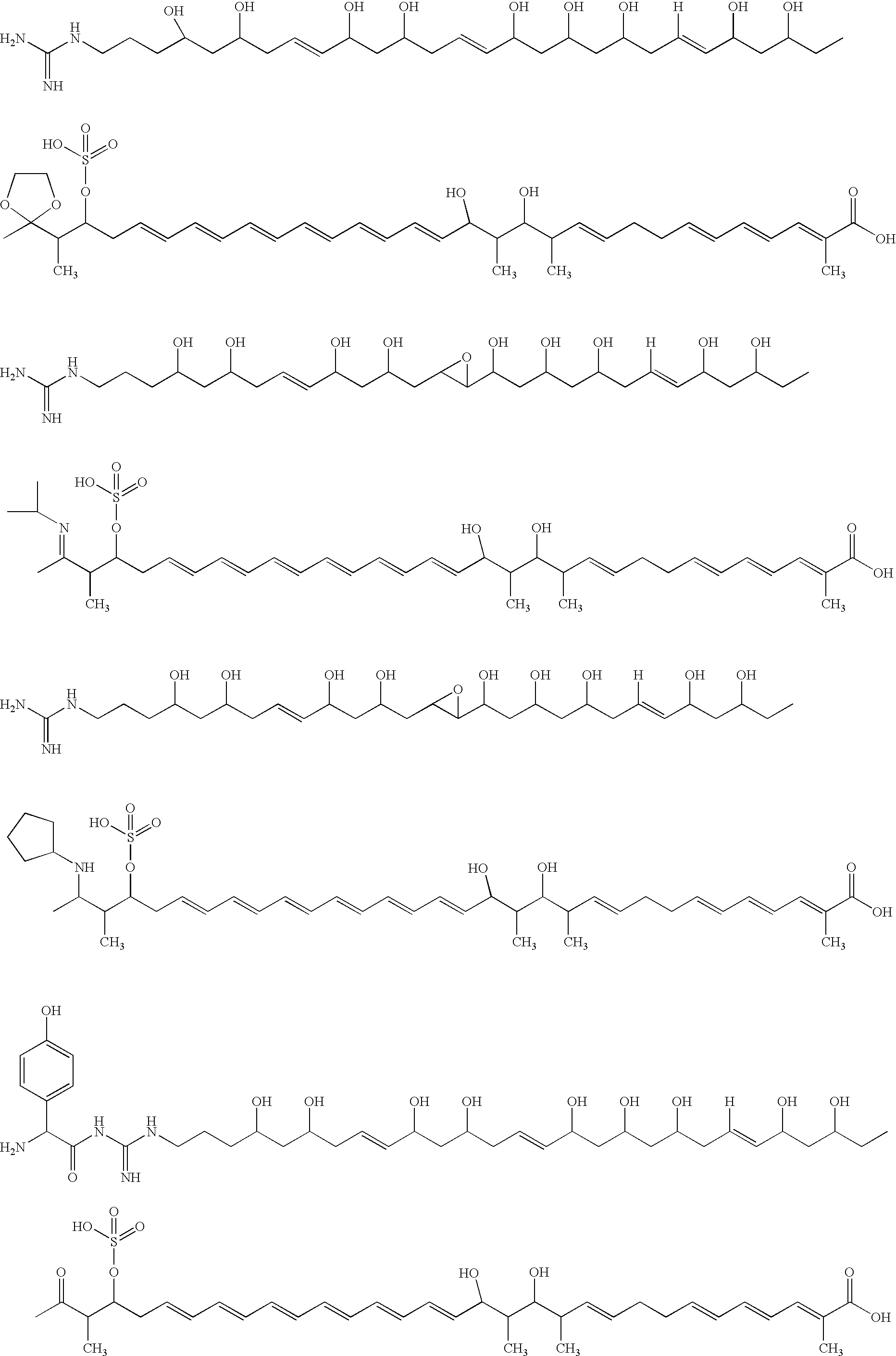

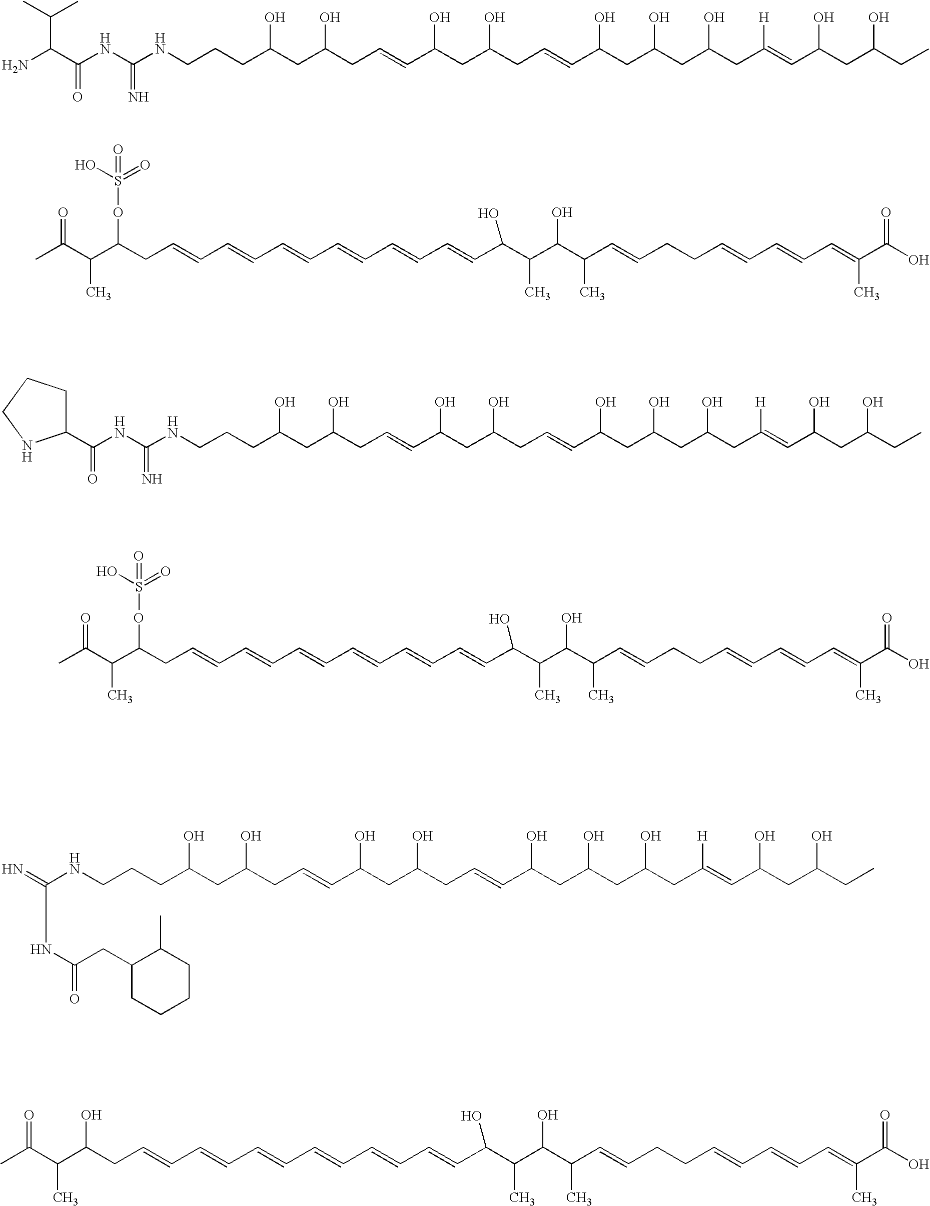

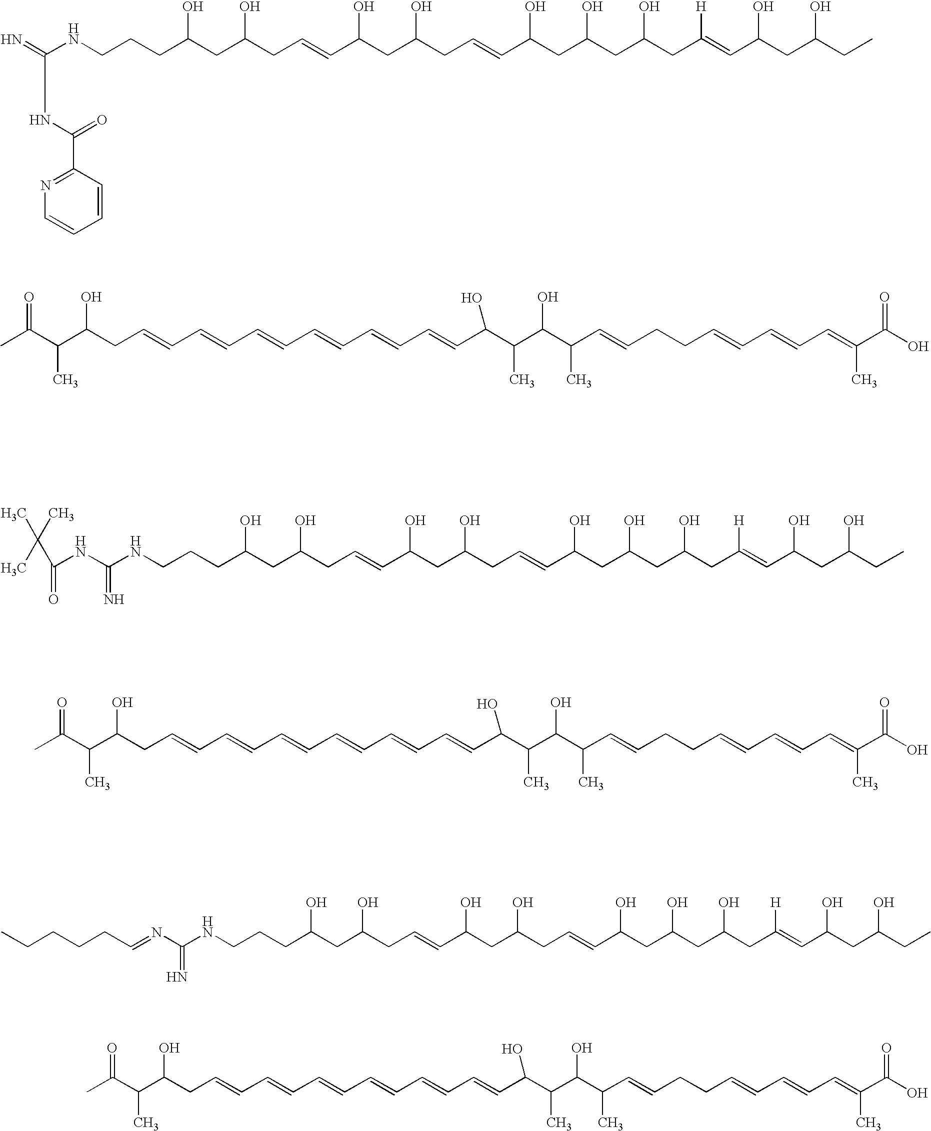

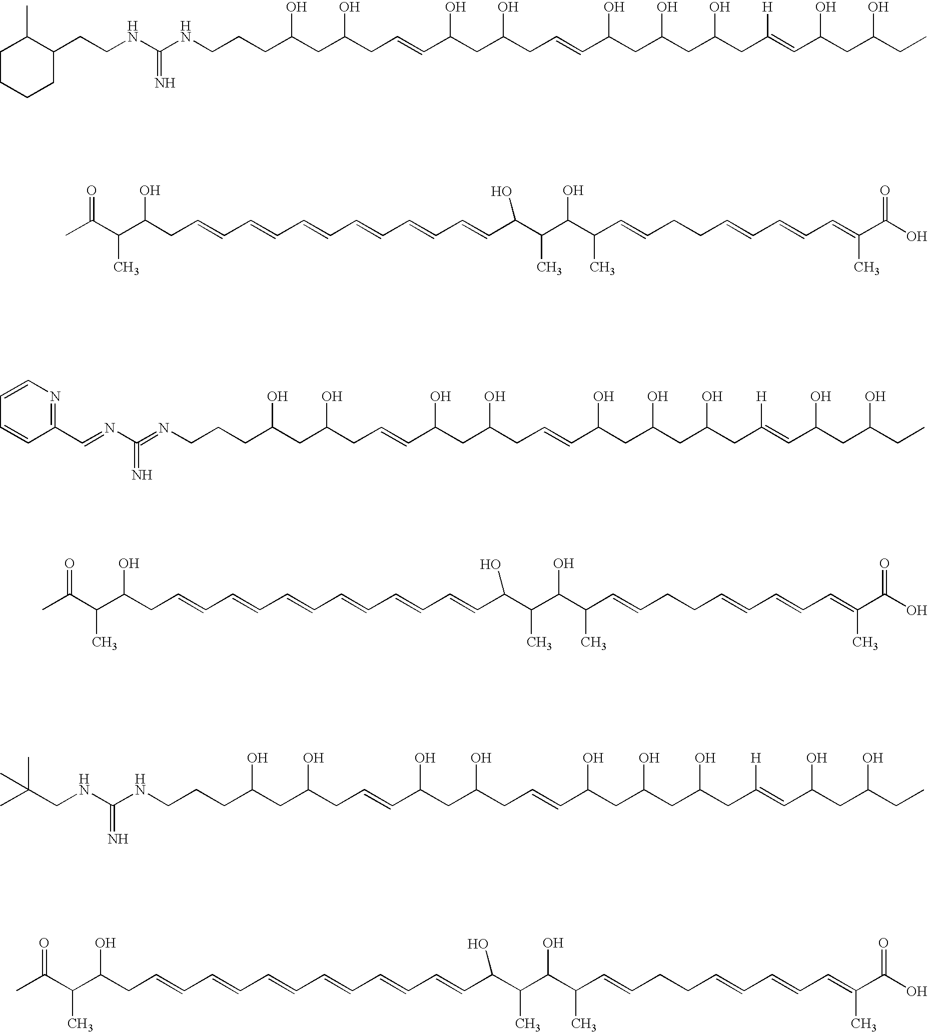

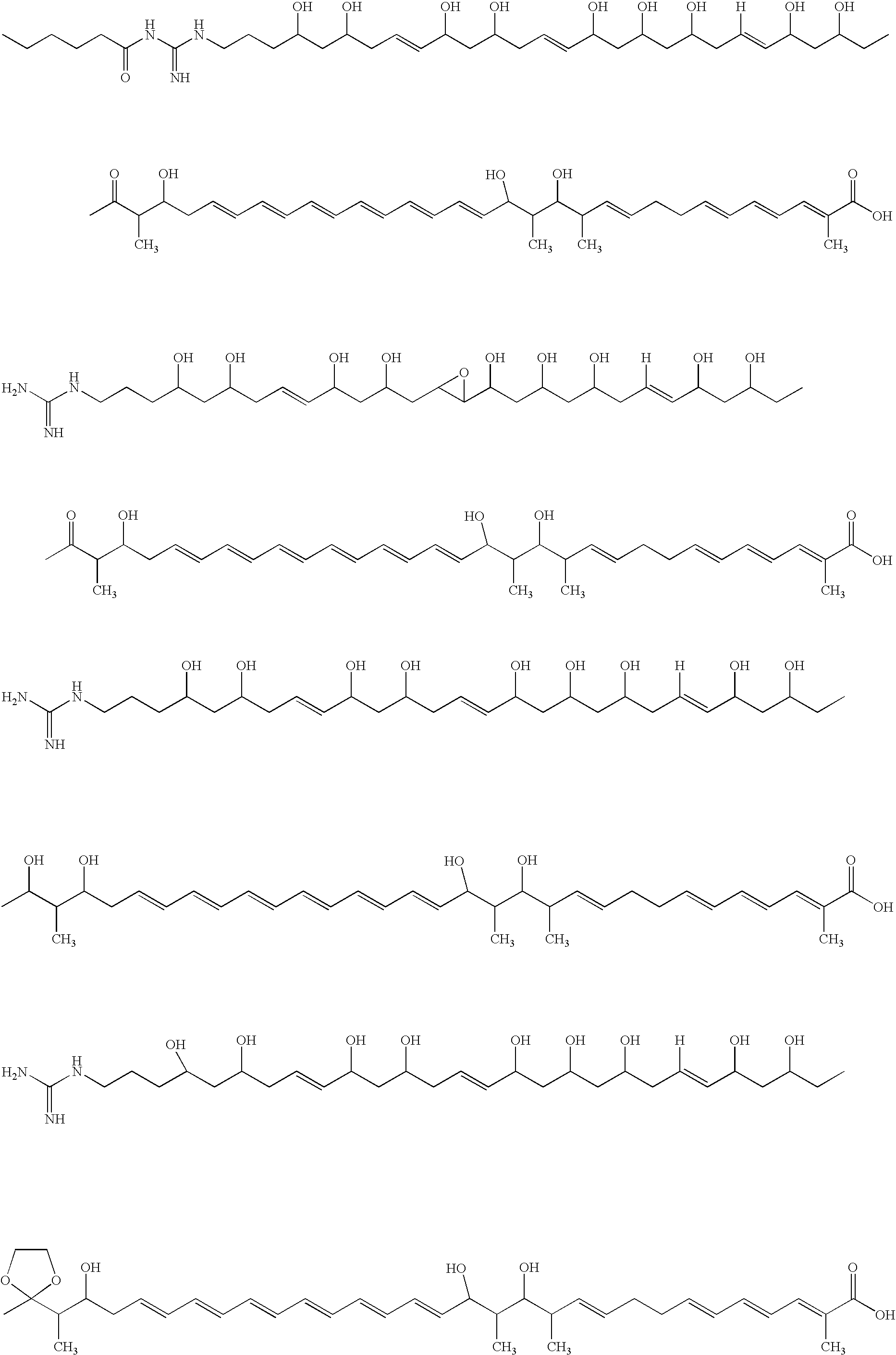

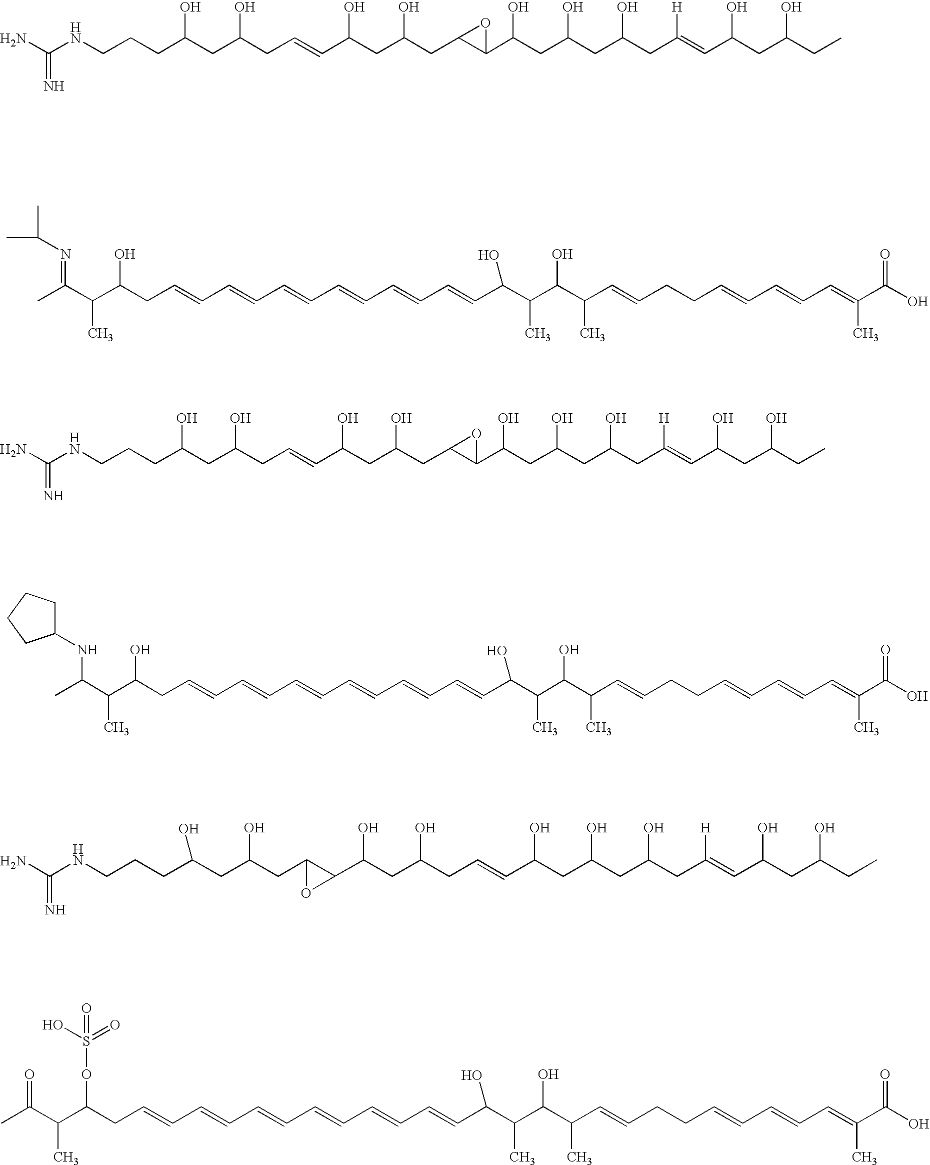

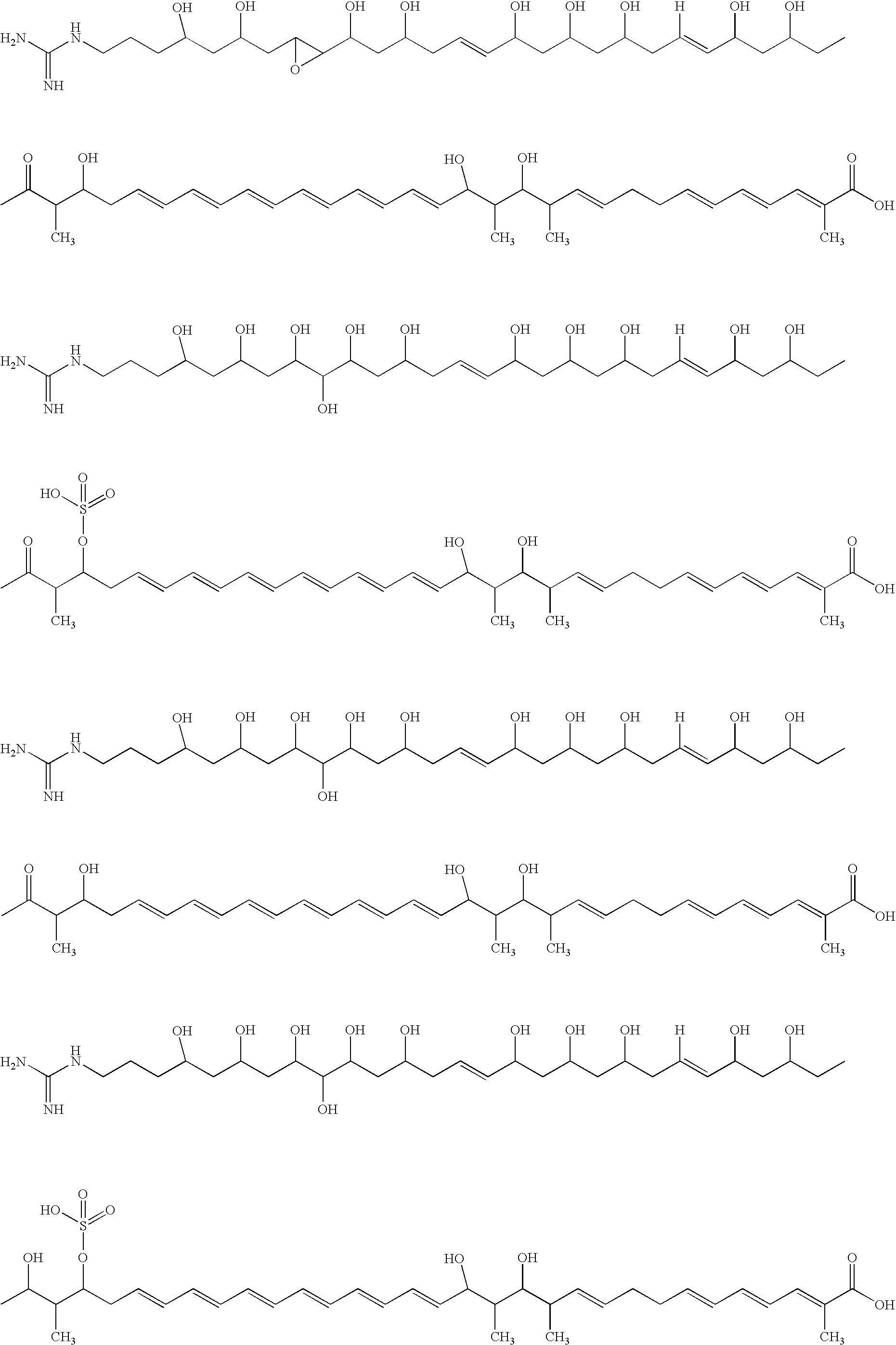

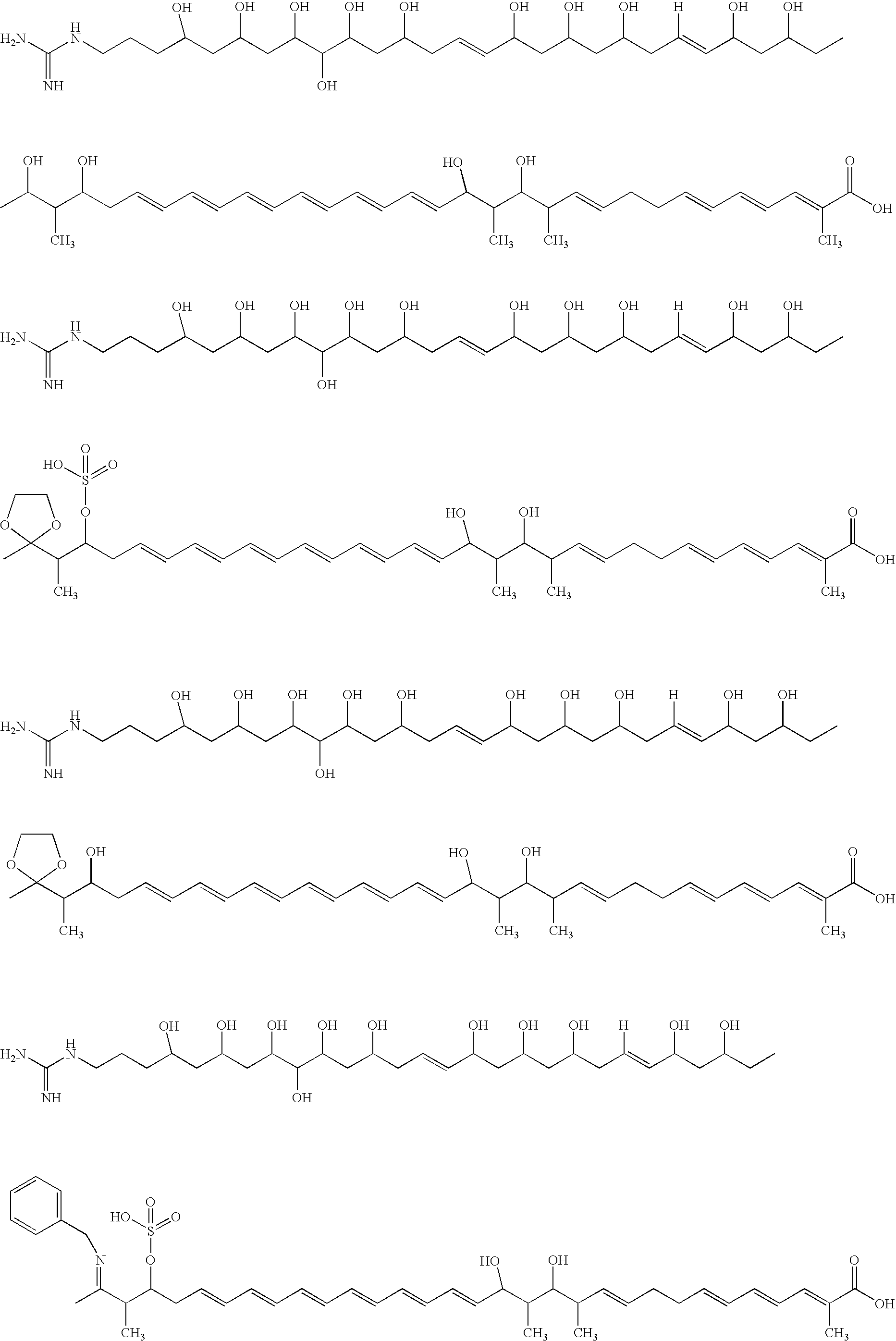

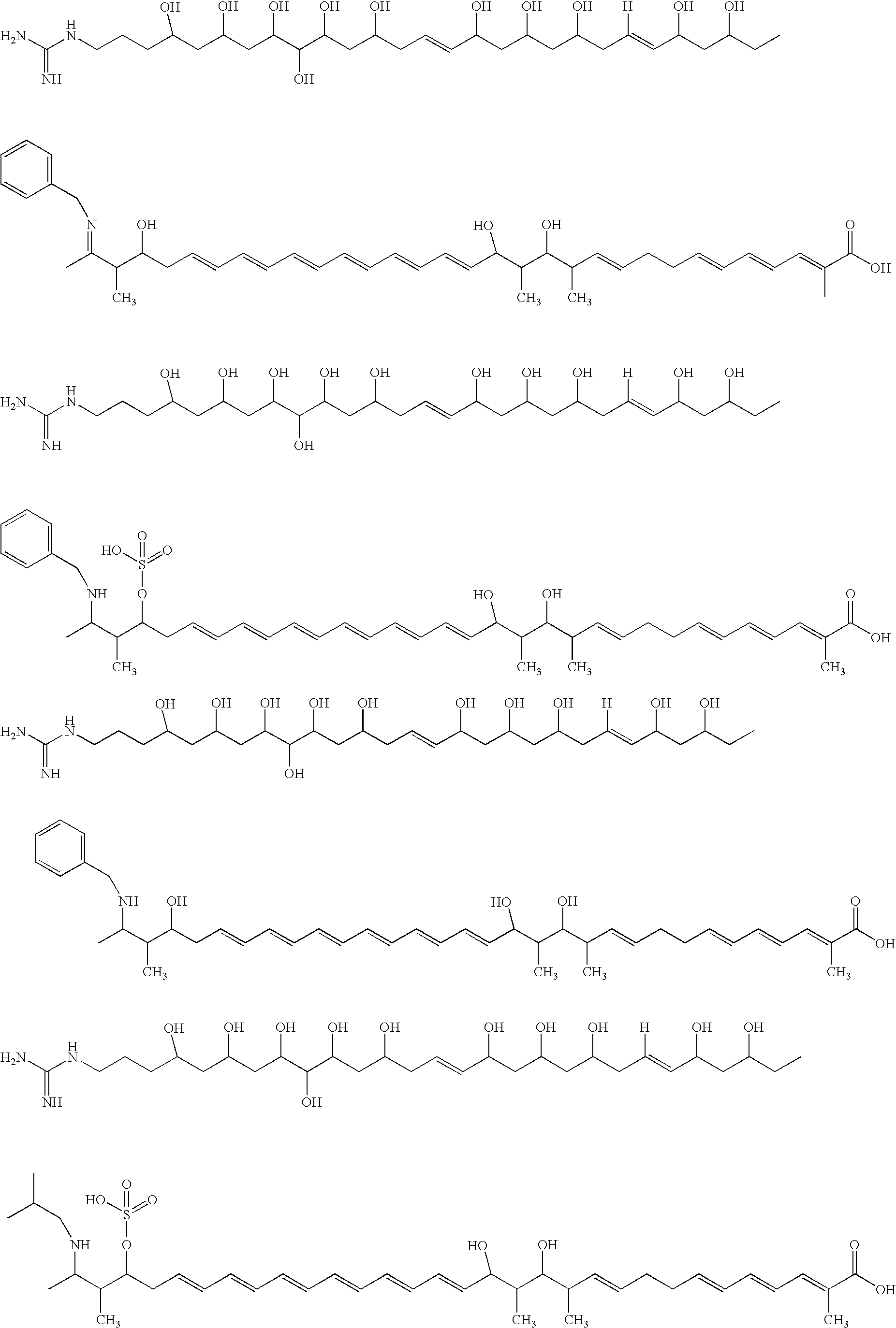

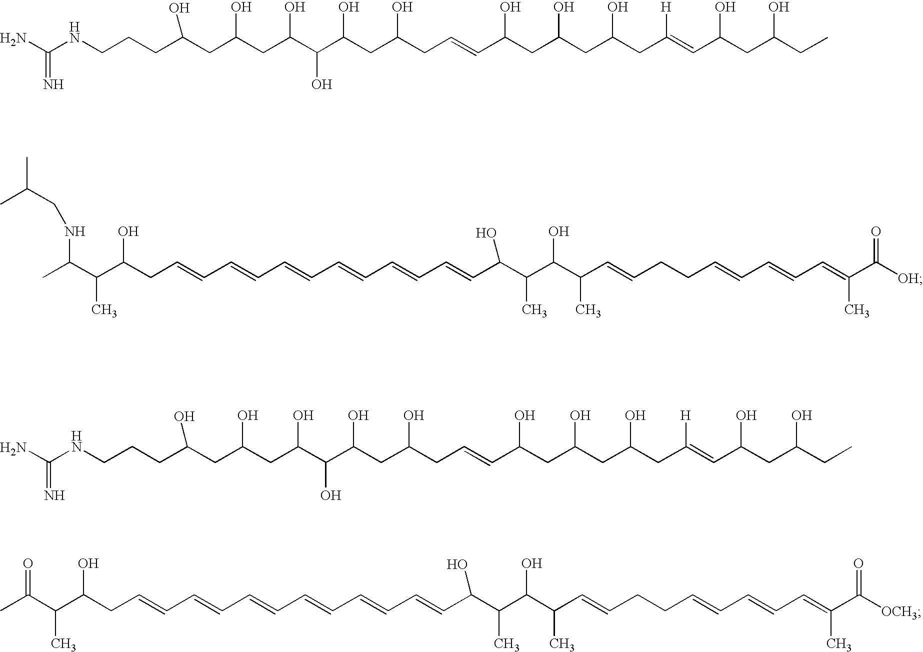

10. A compound selected from the group consisting of: 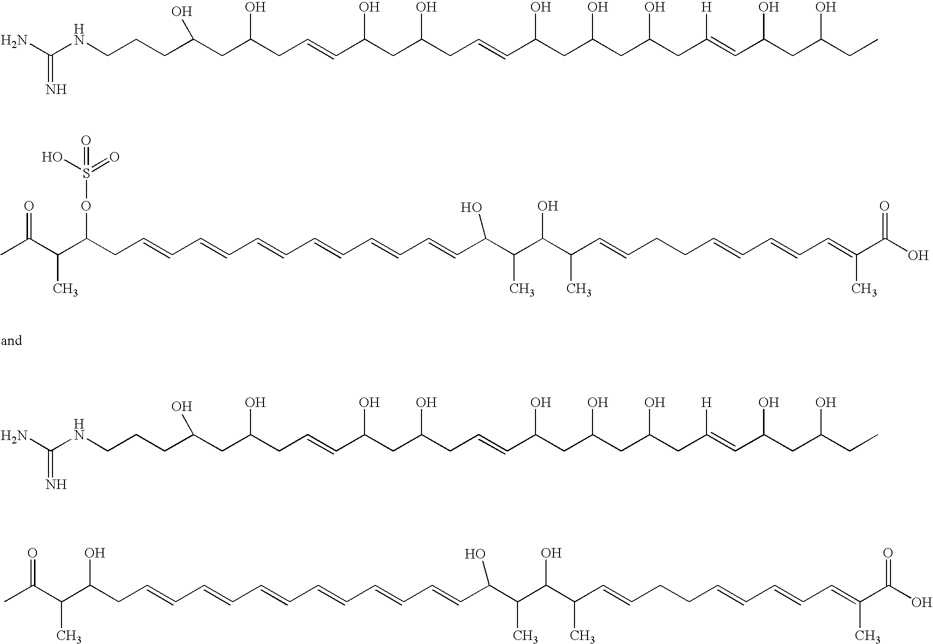
or a pharmaceutically acceptable salt thereof.

11. A method for producing the compound of claim 10, comprising the steps of cultivating cells derived from a Streptomyces melanosporafaciens strain, incubating said cultured cells aerobically in a growth medium comprising at least one source of carbon atoms and at least one source of nitrogen atoms for such time as is required for production of said compound of claim 10, extracting said medium with a solvent and purifying the compound of claim 10 from the crude extract.

12. The method of claim 11 wherein the Streptomyces melanosporafaciens strain is NRRL B-12234 or a mutant thereof.

13. A pharmaceutical composition comprising a therapeutically effective amount of a compound of claim 10 and a pharmaceutically acceptable carrier.

14. A pharmaceutical composition comprising a therapeutically effective amount of compound 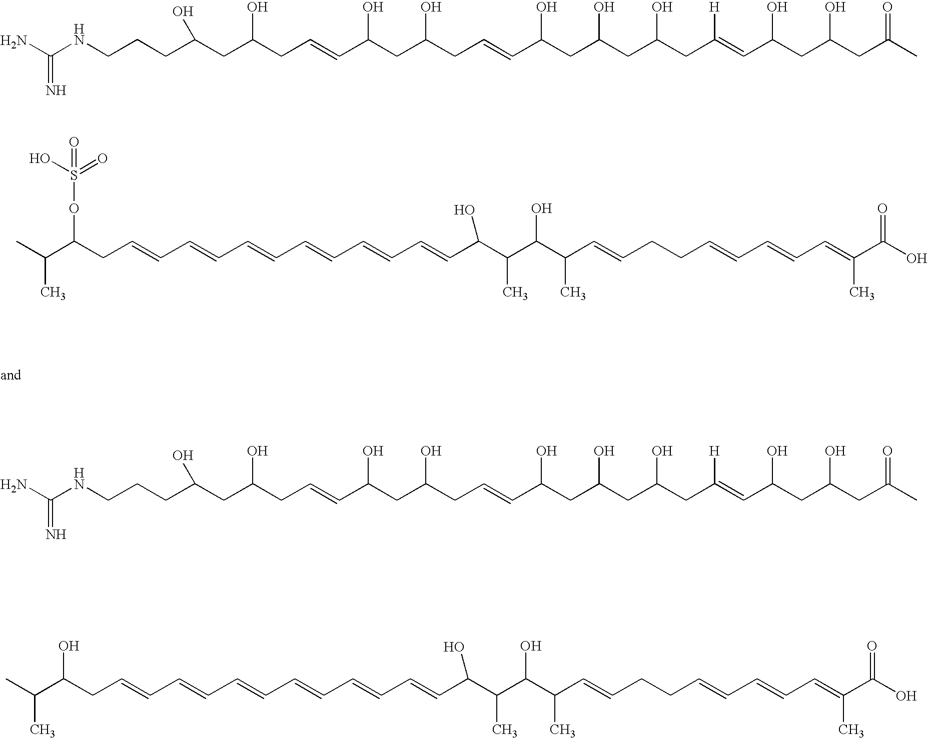
or a pharmaceutically acceptable salt thereof, and a pharmaceutically acceptable carrier.

15. A pharmaceutical composition comprising a therapeutically effective amount of the compound 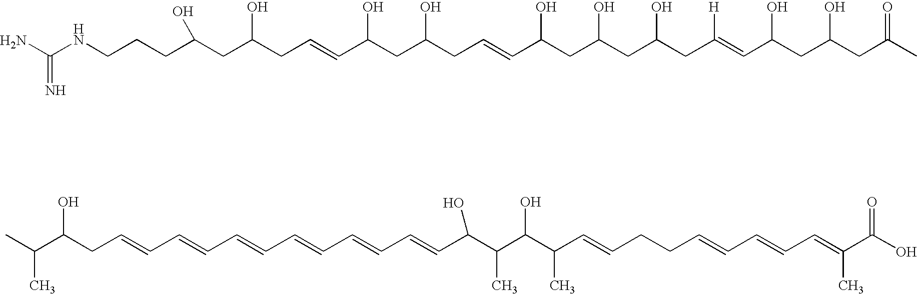
or a pharmaceutically acceptable salt thereof, and a pharmaceutically acceptable carrier.

16. A method of treating tumor growth in a subject, comprising administering to said subject suffering from said tumor growth, a therapeutically effective amount of a compound of claim 10.

17. The method of claim 16, wherein said tumor growth is selected from the group comprising of leukemia, non-small cell lung cancer, colon cancer, CNS cancer, melanoma, ovarian cancer, renal cancer, prostate cancer and breast cancer.

18. A method of treating a fungal infection in a mammal, comprising administering to said mammal suffering from said infection, a therapeutically effective amount of a compound of claim 10.

19. The method of claim 18, wherein said fungal infection is caused by Candida albicans.

20. The method of claim 18, wherein said fungal infection is caused by a Candida sp., wherein said Candida sp. is selected from the group consisting of C. glabrata, C. lusitaniae C. parapsilosis, C. krusei and C. tropicalis.

21. The method of claim 20, wherein said fungal infection is caused by Fusarium spp.; Scedosporium spp.; Cryptococcus spp.; Mucor ssp.; Histoplasma spp.; Trichosporon spp.; Blastomyces spp.; or S. cerevisiae.

22. A method of treating a fungal infection in a subject, comprising administering to said subject suffering from said infection, a therapeutically effective amount of a compound of any one of claims 1 to 10.

23. The method of claim 22, wherein said fungal infection is caused by a fungus selected from the group consisting of Candida albicans, Candida sp., Aspergillus sp., Fusarium spp.; Scedosporium spp.; Cryptococcus spp.; Mucor ssp.; Histoplasma spp.; Trichosporon spp.; Blastomyces spp.; and S. cerevisiae.

24. The method of claim 22, wherein said Candida sp. is selected from the group consisting of C. glabrata, C. lusitaniae, C. parapsilosis, C. krusei and C. tropicalis.
